(19) United States
(12) Patent Application Publication
(10) Pub. No.: US 2024/0139055 A1
(43) Pub. Date: May 2, 2024

(54) SYSTEMS, METHODS AND APPARATUS FOR AUGMENTED ACTIVITY

(71) Applicant: Dephy, Inc., Maynard, MA (US)

(72) Inventors: Luke Mooney, Sudbury, MA (US); Jean-François Duval, Belmont, MA (US); Rachel Harris, Cambridge, MA (US); Jonathan Cummings, Concord, MA (US); William Marvin, Canton, MA (US); Shayna Milstein, Somerville, MA (US)

(73) Assignee: Dephy, Inc., Maynard, MA (US)

(21) Appl. No.: 18/409,536

(22) Filed: Jan. 10, 2024

Related U.S. Application Data
(63) Continuation of application No. PCT/US2022/037083, filed on Jul. 14, 2022.
(60) Provisional application No. 63/222,832, filed on Jul. 16, 2021.
(60) Provisional application No. 63/290,561, filed on Dec. 16, 2021.

(51) Int. Cl. A43B 13/28; A43B 3/26; A43B 3/34

(52) U.S. Cl. CPC A43B 13/28; A43B 3/26; A43B 3/34

(57) ABSTRACT

Systems and methods for integrating bipedalism augmentation components are provided. A system can receive a request to modify a footwear, comprising a sole formed of an insole and an outsole. The system can select a footplate based on a size of the footwear. The footplate can include a first portion extending along at least a first portion of the sole, a second portion extending along at least a second portion of the sole, wherein the first portion of the footplate has greater rigidity than the second portion, and a mounting component connected to the first portion. The system can provide an indication of the selected the footplate for insertion between the insole and the outsole such that the first portion is located proximate to a heel of the footwear and the mounting component protrudes at least partially from the sole, the mounting component configured to couple to a bracket.

### CROSS-REFERENCES TO RELATED APPLICATIONS

This application claims the benefit of priority under 35 U.S.C. § 120 as a continuation of International Patent Application No. PCT/US2022/037083, filed Jul. 14, 2022 and designating the United States, which claims the benefit or priority under 35 U.S.C. § 119 to U.S. Provisional Patent Application No. 63/222,832, filed on Jul. 16, 2021, titled “SYSTEMS, METHODS AND APPARATUS FOR AUGMENTED RUNNING,” and U.S. Provisional Patent Application No. 63/290,561, filed on Dec. 16, 2021, titled “SYSTEMS, METHODS AND APPARATUS FOR AUGMENTED RUNNING,” all of which are hereby incorporated herein by reference in their entirety.

### BACKGROUND

Exoskeletons can be worn by a user to facilitate the movement of limbs of the user.

### SUMMARY

An aspect of this disclosure can be directed to a method. The method can include providing footwear comprising a sole formed of an insole and an outsole. The method can include selecting, based on a size of the footwear, a footplate having a length that is less than the size of the footwear. The footplate can comprise a first portion of the footplate to extend along at least a first portion of the sole, a second portion of the footplate to extend along at least a second portion of the sole. The first portion of the footplate can have greater rigidity than the second portion of the footplate. The method can include a mounting component connected to the first portion of the footplate. The method can include inserting the footplate between the insole and the outsole of the footwear such that the first portion of the footplate with greater rigidity is located proximate to a heel of the footwear and the mounting component protrudes at least partially from the footwear. The method can include coupling a bracket to the mounting component, the bracket configured to receive force from an actuator and apply the force to the first portion of the footplate via an axis of rotation about an ankle to augment motion.

An aspect of this disclosure can be directed to a system. The system can include a data processing system comprising memory and one or more processors. The one or more processors can receive a request to modify a footwear for augmentation. The footwear can include a sole formed of an insole and an outsole. The one or more processors can select a footplate based on a size of the footwear to have a length that is less than the size of the footwear. The footplate can include a first portion of the footplate to extend along at least a first portion of the sole. The footplate can include a second portion of the footplate to extend along at least a second portion of the sole. The first portion of the footplate can have greater rigidity than the second portion of the footplate. The system can include a mounting component connected to the first portion of the footplate. The one or more processors can provide an indication of the selected the footplate for insertion between the insole and the outsole of the footwear such that the first portion of the footplate with greater rigidity is located proximate to a heel of the footwear and the mounting component protrudes at least partially from the sole. The mounting component can be configured to couple to a bracket to receive force from an actuator and apply the force to the first portion of the footplate via an axis of rotation about an ankle to augment motion.

An aspect of this disclosure can be directed to a system. The system can include a shin pad configured to couple to a shin of a subject below a knee of the subject. The system can include a battery pack comprising a plurality of battery cells; a footplate inserted in a sole of footwear; a housing, coupled to the shin pad, that encloses an electric motor configured to apply force via a lever to the footplate to generate torque about an axis of rotation of an ankle joint of the subject. The system can include a controller comprising one or more processors and memory, electrically connected with the electric motor and the battery pack. The controller can determine a performance profile for the electric motor based on a number of battery cells in the plurality of battery cells or a type of battery cell in the plurality of battery cells. The controller can establish a duty cycle for a switched-mode power supply to cause the switched-mode power supply to convey power from the battery pack to the electric motor to generate the torque about the axis in accordance with the determined performance profile.

An aspect of this disclosure can be directed to a method. The method can include providing a shin pad configured to couple to a shin of a subject below a knee of the subject. The method can include providing a battery pack comprising a plurality of battery cells. The method can include providing a footplate inserted in a sole of footwear; providing a housing, coupled to the shin pad, that encloses an electric motor configured to apply force via a lever to the footplate to generate torque about an axis of rotation of an ankle joint of the subject. The method can include determining, by a controller comprising one or more processors and memory, electrically connected with the electric motor and the battery pack, a performance profile for the electric motor based on a number of battery cells in the plurality of battery cells or a type of battery cell in the plurality of battery cells. The method can include establishing, by the controller, a duty cycle for a switched-mode power supply to cause the switched-mode power supply to convey power from the battery pack to the electric motor to generate the torque about the axis in accordance with the determined performance profile.

An aspect of this disclosure can be directed to a system. The system can include a shin pad configured to couple to a shin of a subject below a knee of the subject. The system can include a battery pack. The system can include a footplate inserted in a sole of a footwear. The system can include a housing, coupled to the shin pad, that encloses an electric motor configured to apply force via a lever to the footplate to generate torque about an axis of rotation of an ankle joint of the subject. The system can include a controller comprising one or more processors and memory, electrically connected with the electric motor and the battery pack. The controller can identify one or more characteristics of the footwear in which the footplate is inserted. The controller can receive, via a user interface, an indication of a mode for augmentation. The controller can select a model for torque generation based on the one or more characteristics of the footwear. The controller can instruct the electric motor to generate torque based on the model.

An aspect of this disclosure can be directed to a method. The method can include providing a shin pad configured to couple to a shin of a subject below a knee of the subject. The method can include providing a battery pack. The method can include providing a footplate inserted in a sole of a footwear. The method can include providing a housing, coupled to the shin pad, that encloses an electric motor configured to apply force via a lever to the footplate to generate torque about an axis of rotation of an ankle joint of the subject. The method can include identifying, by a controller comprising one or more processors and memory electrically, connected with the electric motor and the battery pack, one or more characteristics of the footwear in which the footplate is inserted. The method can include receiving, by the controller via a user interface, an indication of a mode for augmentation. The method can include selecting, by the controller, a model for torque generation based on the one or more characteristics of the footwear. The method can include instructing, by the controller, the electric motor to generate torque based on the model.

### BRIEF DESCRIPTION OF THE DRAWINGS

The details of one or more implementations of the subject matter described in this specification are set forth in the accompanying drawings and the description below. Other features, aspects, and advantages of the subject matter will become apparent from the description, the drawings, and the claims.

FIG. 1 illustrates a schematic diagram of a lower limb exoskeleton.

FIG. 2 illustrates a relationship between the human foot and a composite plate design.

FIG. 3 illustrates a relationship between the human foot and a composite plate design.

FIG. 4 illustrates a relationship between the human foot and a composite plate design.

FIG. 5 illustrates a relationship between the human foot and a composite plate design.

FIG. 6 illustrates a bottom sole assembly for footwear.

FIG. 7 illustrates a schematic diagram of a rear mounting point for the exoskeleton.

FIGS. 8A-8C illustrate a metatarsal actuator integrated into footwear sole.

FIGS. 9A-9B illustrate the forefoot actuator.

FIG. 10 illustrates the forefoot actuator.

FIG. 11 illustrates the forefoot actuator.

FIG. 12A illustrates a system powered in metatarsal dorsiflexion.

FIG. 12B illustrates a system powered in metatarsal plantarflexion.

FIG. 13 illustrates a schematic diagram of the rotational axis of the ankle joint.

FIG. 14 illustrates a symmetrical ankle exoskeleton design.

FIG. 15 illustrates a symmetrical linkage system.

FIG. 16 illustrates an elastic ankle lever.

FIG. 17 illustrates an elastic exoskeleton upright element.

FIG. 18 illustrates a motor located behind the leg/calf.

FIG. 19 illustrates a motor located behind the leg/calf.

FIG. 20 illustrates a motor located behind the leg.

FIG. 21A-21B illustrate a motor located in front of leg/shin.

FIG. 22 illustrates a composites sheet construction.

FIG. 23 illustrates an exoskeleton.

FIG. 24 illustrates an exoskeleton.

FIG. 25 illustrates an around the corner transmission 4 bar linkage.

FIG. 26 illustrates a hydraulic transmission.

FIG. 27 illustrates a hydraulic actuator.

FIG. 28 illustrates a hydraulic actuator

FIG. 29 illustrates a 4 bar lateral linkage.

FIG. 30 illustrates a 4 bar linkage 90° transmission exoskeleton.

FIG. 31 illustrates a carbon fiber underfoot structure.

FIGS. 32A-32B illustrate a combined ankle and metatarsal actuation system.

FIG. 33A illustrates a back mount battery attachment.

FIG. 33B illustrates a chest mount battery attachment.

FIG. 33C illustrates a rear waist battery attachment.

FIG. 33D illustrates a front waist battery attachment.

FIG. 33E illustrates a back mount battery attachment.

FIG. 33F illustrates a chest mount battery attachment.

FIG. 33G illustrates a thigh mount battery attachment.

FIG. 33H illustrates a hip mount battery attachment.

FIG. 33I illustrates a front waist battery attachment.

FIG. 33J illustrates a rear waist battery attachment.

FIG. 34 illustrates a battery module.

FIG. 35 illustrates a battery module and attachment.

FIG. 36 illustrates a centering mechanism.

FIG. 37 illustrates a centering mechanism.

FIG. 38 illustrates a centering mechanism.

FIG. 39 illustrates a centering mechanism.

FIG. 40 illustrates a bionic shoe soft actuator.

FIG. 41 illustrates a bionic shoe.

FIG. 42 illustrates a bionic shoe.

FIG. 43 illustrates shin levers and quick-disconnects.

FIGS. 44A-B illustrate a battery module.

FIG. 45 is a block diagram of a system for augmenting motion.

FIG. 46 is an example flow diagram of a method for a footwear attachment process.

FIG. 47 is an example flow diagram of a method for battery and hardware for augmented motion.

FIG. 48 is an example flow diagram of a method for software and controller for augmented motion.

Like reference numbers and designations in the various drawings indicate like elements.

### DETAILED DESCRIPTION

This disclosure relates generally to performance enhancing wearable technologies. Particularly, this disclosure relates to apparatuses, systems, and methods for augmented running. An exoskeleton can augment a user's activities, such as running. Rigid and compliant structures can be integrated directly into footwear. This can allow for benefits for the purposes of human augmentation. For example, these structures can include a way to apply force to the ankle and lower limbs without injury or discomfort. An engineered composite structure under the foot with a rigid mounting point for the mechanical exoskeleton can be used. This can be expanded to further integrate the compliant and rigid structures found in footwear designs to stabilize and support the foot with the structures needed to support and attach to the ankle exoskeleton. These structures can be optimized for attributes such as high strength, lower mass, robustness, and elasticity.

### I. Exoskeleton Overview

Exoskeletons (e.g., lower limb exoskeleton, knee exoskeleton, back exoskeleton, etc.) can include devices worn by a person to augment physical abilities. Exoskeletons can be considered passive (e.g., not requiring an energy source such as a battery) or active (e.g., requiring an energy source to power electronics and usually one or many actuators). Exoskeletons may be capable of providing large amounts of force, torque and/or power to the human body in order to assist with motion.

Exoskeletons can transfer energy to the human and may not interfere with the natural range of motion of the body. Exoskeletons can convert the energy source into useful mechanical force, torque, or power. Onboard electronics (e.g., controllers) can control the exoskeleton. Output force and torque sensors can also be used to make controlling easier.

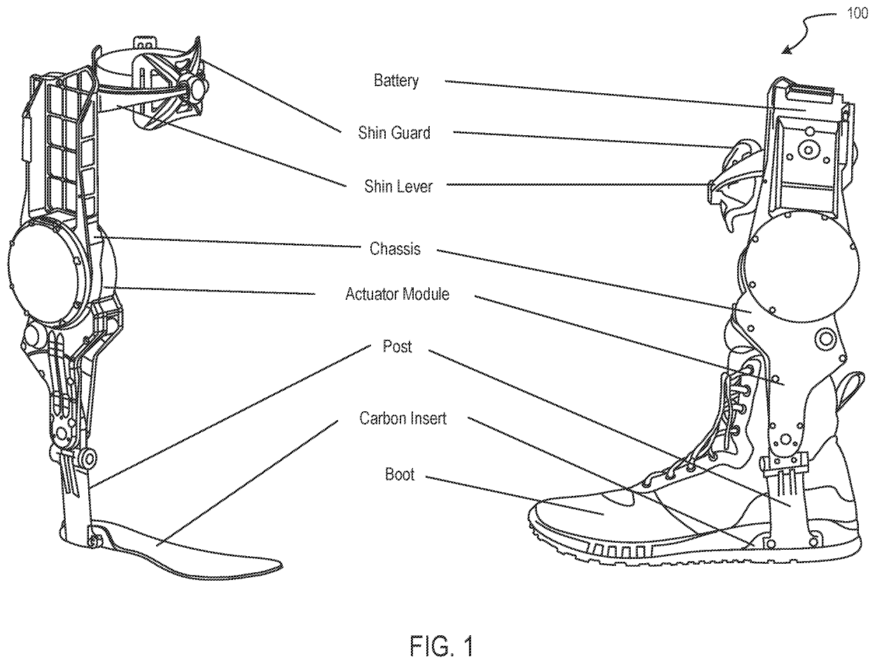

FIG. 1 illustrates a schematic diagram of a lower limb exoskeleton 100 (e.g., lower limb exoskeleton assembly, lower limb exoskeleton system, exoskeleton boot, mechanical exoskeleton, exoskeleton device, etc.). The exoskeleton 100 can include a battery that can include an assembly that is installed onto the actuator module and supplies electrical energy to the system. The exoskeleton 100 can include a shin guard that can include a part of the assembly that interfaces with the user's shin. The exoskeleton 100 can include a shin lever that can include a mechanical structure that connects the shin guard to a chassis. The chassis can include a mechanical structure that connects the static components. The exoskeleton 100 can include an actuator module that can include all the component in the lower limb exoskeleton assembly excluding the boot. The exoskeleton 100 can include a post that can include a mechanical structure that connects to the boot. The exoskeleton 100 can include a carbon insert that can include a carbon fiber structure located inside of the sole of the boot. The exoskeleton 100 can include a boot that connects to the user and the actuator module. The exoskeleton 100 can include a spool shaft that can include a shaft that is driven by the motor and winds the belt around itself. The belt can include a tensile member that is pulled by the spool shaft and applies a force to the ankle lever. The exoskeleton 100 can include an ankle lever that can include a lever used to transmit torque to the ankle. Lower limb exoskeletons can be used to augment the ankle joint.

The lower limb exoskeleton can include a rugged system used for field testing. The lower limb exoskeleton can include an integrated ankle lever guard (e.g., nested lever). The lower limb exoskeleton can include a mechanical shield to guard the belt and ankle lever transmission from the environment. The housing structure of the system can extend to outline the range of travel of the ankle lever on the lateral and medial side. The lower limb exoskeleton can include a shin lever self-centering mechanism. A self-centering mechanism can be incorporated into the shin lever. Degrees of freedom can be incorporated into the lower limb exoskeleton to reduce skin sheer and increase the comfort to the user. The lower limb exoskeleton can include a self-centering mechanism to push the shin lever to the shin lever's center of travel if the shin lever is not already there. This mechanism can be composed of one or more springs. The self-centering mechanism on the lower limb exoskeleton can use repelling magnets to push the shin lever to its center of travel. The magnets can be attracted each other and pull the shin lever to its center of travel.

### II. Augmented Running

According to the systems and methods of the present disclosure, an exoskeleton can augment a user's activities, such as running. Rigid and compliant structures can be integrated directly into footwear. This can allow for benefits for the purposes of human augmentation. For example, these structures can include a way to apply force to the ankle and lower limbs without injury or discomfort. Design constraints can include mechanical interference between the device, joint or limbs and avoidance of sensitive, and or highly flexible areas on the lower leg and foot. An engineered composite structure under the foot with a rigid mounting point for the mechanical exoskeleton can be used. This can be expanded to further integrate the compliant and rigid structures found in footwear designs to stabilize and support the foot with the structures needed to support and attach to the ankle exoskeleton. These structures can be optimized for attributes such as high strength, lower mass, robustness, and elasticity.

In some embodiments, the exoskeleton can include a composite plate integrated into the sole of an article of footwear. The composite plate can provide a rigid mounting point for an ankle exoskeleton and reaction plate to translate forces to the ground for the purposes of augmenting human movement. A composite underfoot structure can be located under the foot. The underfoot structure can be layered between layers of cushioning material (e.g., ethylene-vinyl acetate (EVA) foam, polyurethane (PU) foam, etc.). The underfoot structure can be a full length underfoot structure (e.g., from the toe area to the heel area) or a partial underfoot structure (e.g., from the metatarsal flex area to the heel area).

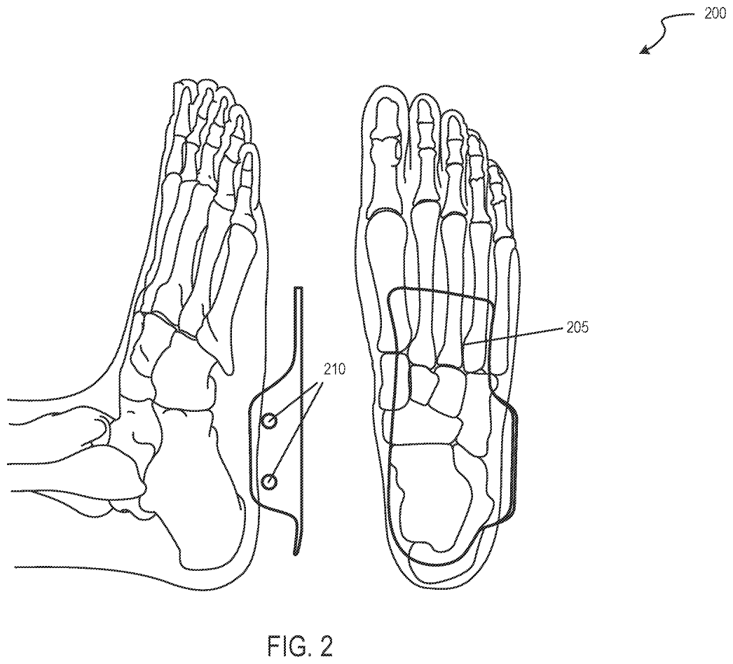

FIG. 2 illustrates a relationship 200 between the human foot and a composite plate design. The composite plate 205 (e.g., semi-rigid composite plate, composite footplate, footplate, etc.) can terminate at the approximate location behind the metatarsal joints. The composite plate 205 can span the length between the area behind metatarsal joints and the heel area. The composite plate 205 can include mounting holes 210 (e.g., a plurality of mounting holes). The exoskeleton 100 can be coupled with the composite plate 205 at the mounting holes 210.

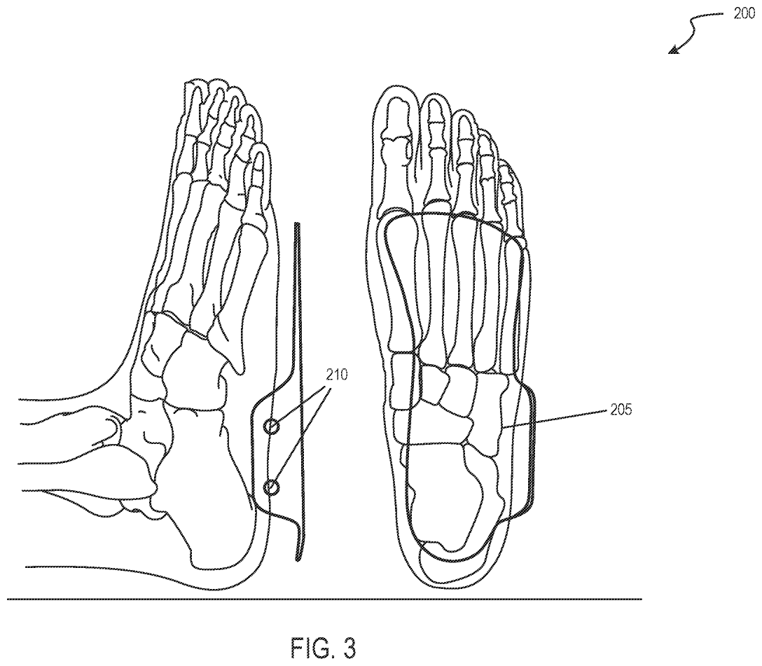

FIG. 3 illustrates the relationship 200 between the human foot and a composite plate design. The composite plate 205 can terminate at the approximate location of the metatarsal joints. The composite plate 205 can span the length between the metatarsal joints and the heel area. The composite plate 205 can include the mounting holes 210. The exoskeleton 100 can be coupled with the composite plate 205 at the mounting holes 210.

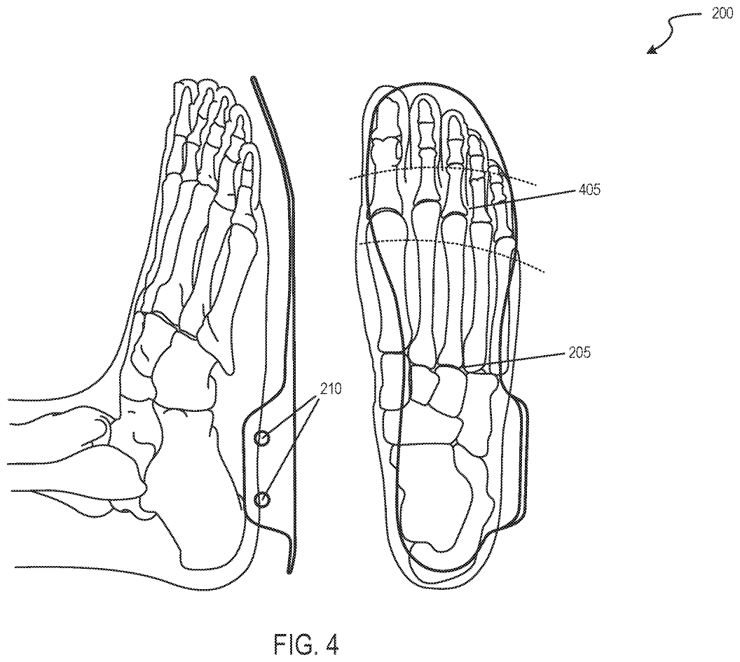

FIG. 4 illustrates the relationship 200 between the human foot and a composite plate design. The composite plate 205 can span the full length of the foot. For example, the composite plate 205 can span the length between the toe areas to the heel area. The composite plate 205 can include the mounting holes 210. The exoskeleton 100 can be coupled with the composite plate 205 at the mounting holes 210. The relationship 200 can include the location of the metatarsal joints 405. The location of the metatarsal joints 405 can include an approximate location of the metatarsal joints 405.

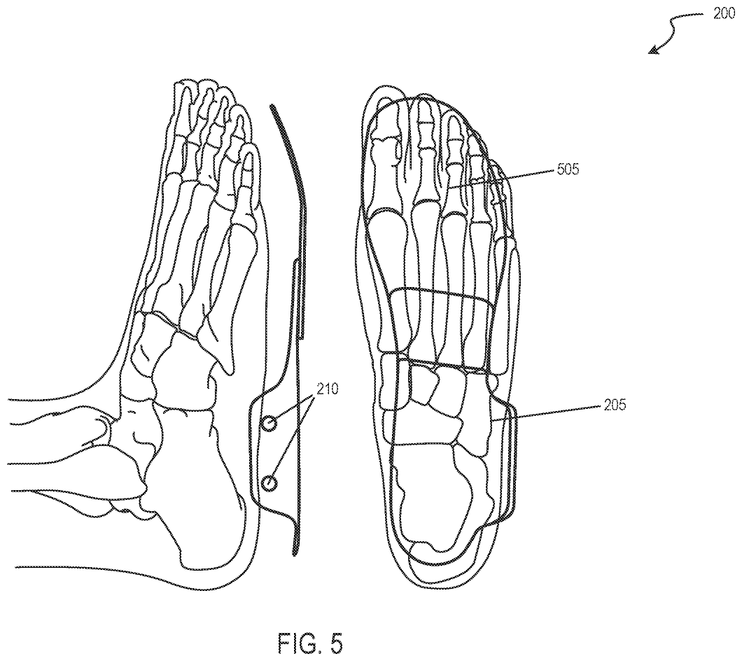

FIG. 5 illustrates the relationship 200 between the human foot and a composite plate design. The composite plate 205 can span the full length of the foot. For example, the composite plate 205 can span the length between the toe areas to the heel area. The composite plate 205 can include the mounting holes 210. The exoskeleton 100 can be coupled with the composite plate 205 at the mounting holes 210. The composite plate 205 can include a thin, compliant and/or unidirectional composite material 505 under the forefoot. The composite material 505 can be mechanically attached to the rigid heel section.

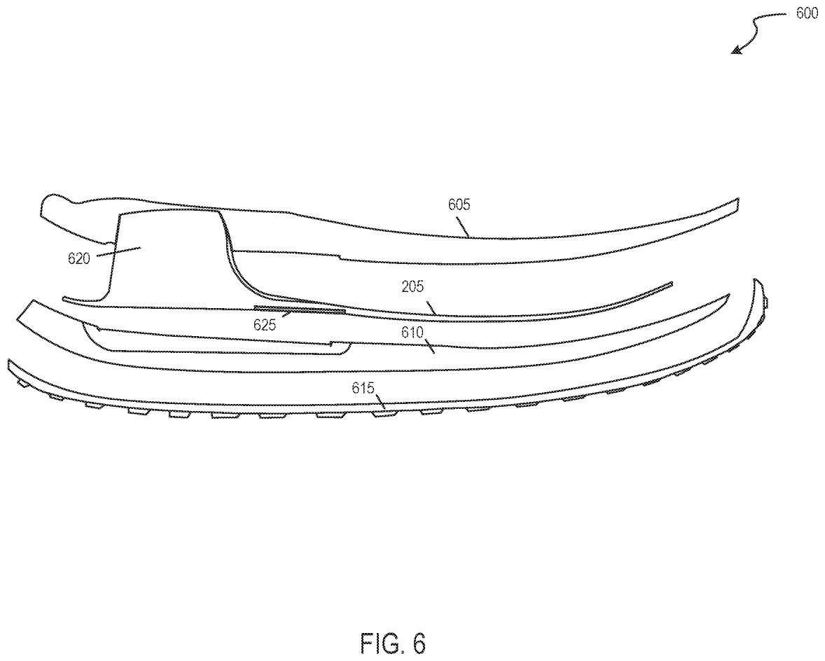

FIG. 6 illustrates a bottom sole assembly for footwear 600. The footwear 600 can include a sole (e.g., bottom sole). The bottom sole assembly for footwear 600 can include an upper cushioning thickness 605. The bottom sole assembly for footwear 600 can include the composite plate 205. The bottom sole assembly for footwear 600 can include a lower cushioning thickness 610. In some cases, the upper cushioning thickness 605 of the bottom sole can be referred to as an insole of the footwear 600. The lower cushioning thickness 610 of the bottom sole can be referred to as an outsole of the footwear 600. The composite plate 205 can be inserted, placed, or formed between the upper cushioning thickness 605 and the lower cushioning thickness 610 of the footwear 600. For example, the composite plate 205 can be inserted into the bottom sole assembly, such that the composite plate 205 is interposed between the insole and the outsole of the footwear 600, as shown in FIG. 6.

The composite plate 205 can include a size or dimension based on the size of the footwear 600. The size of the composite plate 205 can be selected, for example, by a computing device executing a program, script, or instructions. The size of the composite plate 205 can be selected to have a dimension of less than the dimension of the footwear. For example, at least one of the length, width, etc. of the composite plate 205 may be less than (or in some cases, equal to) the length, width, etc. of the footwear 600.

The composite plate 205 can be inserted between the insole and the outsole of the footwear 600 subsequent to manufacturing or construction of the footwear 600. In some cases, the composite plate 205 can be inserted between the insole and the outsole as part of the manufacturing operation or process of assembling the footwear 600. The composite plate 205 can include, correspond to, or be a part of a footplate. For example, the composite plate 205 (e.g., footplate) can include one or more portions extending along at least a respective portion of the sole of the footwear 600. The one or more portions of the composite plate 205 can include similar or different levels (or amounts) of rigidity, types of materials (e.g., composition), etc. The one or more portions of the composite plate 205 can be stacked (e.g., on top of each other), side by side, or any combination thereof.

For example, as shown in FIG. 5, the composite plate 205 can include the composite material 505, which can be a part of the footplate. In this case, the composite plate 205 may be associated with a first portion of the footplate, and the composite material 505 may be associated with a second portion of the footplate. The first portion of the footplate can extend along at least a first portion of the sole, and the second portion of the footplate can extend along at least a second portion of the sole. The second portion of the footplate may at least partially overlap the first portion of the footplate or vice versa. The first portion and the second portion of the footplate can include different amounts of rigidity. For instance, the first portion of the footplate (e.g., the portion located proximate to a heel of the footwear 600) can include greater rigidity relative to or compare to the second portion of the footplate or vice versa. In some cases, the first and second portions of the footplate can include a similar amount of rigidity.

The first and second portions of the footplate can include different thicknesses. In some cases, the second portion of the footplate can be thinner than the first portion of the footplate. In some other cases, the first portion can be thinner than the second portion of the footplate. In certain aspects, the one or more portions of the footplate may include a similar thickness.

The bottom sole assembly for footwear 600 can include a rubber outsole 615. The rubber outsole 615 can be a part of the sole of the footwear 600. For example, the rubber outsole 615 may be a part of the outsole. In some cases, the footplate can be inserted between the lower cushioning thickness 610 and the rubber outsole 615. The bottom sole assembly for footwear 600 can include a mounting tab 620. The bottom sole assembly for footwear 600 can include an integrated strain gauge 625.

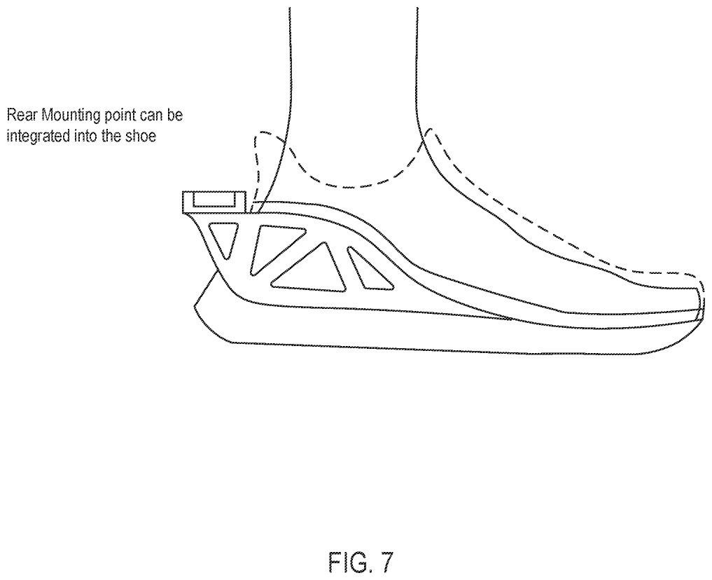

The mounting tab 620 can be a part of the footplate. In some cases, the mounting tab 620 can correspond to or be a part of a mounting component (or a mounting portion) of the footplate. The mounting tab 620 can be integrated as part of the footplate or attached to the footplate. For example, the mounting tab 620 (e.g., mounting component) can be connected, embedded, or coupled to a portion of the footplate (e.g., the first portion of the footplate located proximate to the heel of the footwear 600). The mounting component can be located to the side of the footplate (e.g., mounting tab 620 shown in FIG. 6). In some cases, the mounting component can be located to the rear, back, or behind the footplate (e.g., a rear mounting point shown in FIG. 7). The mounting tab 620 or the mounting component can be referred to as an attachment point for coupling or connection to the exoskeleton 100. The mounting component can protrude at least partially from the footwear 600. The mounting component can protrude from the rear of the footwear 600. The mounting component can protrude from at least one side (e.g., at the heel) of the footwear 600 different from the rear of the footwear 600. The mounting component can be composed of similar or different materials from the footplate, such as carbon fiber, plastic, metal, etc., suitable to support the coupling with the exoskeleton 100.

The exoskeleton 100 can be coupled with the bottom sole assembly for footwear 600 behind the heel of the shoe at an attachment point. The attachment point can include a point of rigid attachment. The attachment point can include, correspond to, or be a part of the mounting tab 620 connected to the composite plate 205. In some cases, the attachment point can be located behind the foot (or the footplate) to interface with the rest of the exoskeleton 100. In some other cases, the attachment point can be located proximate to at least one of the sides of the footplate (e.g., adjacent to the first portion of the footplate) to interface with the exoskeleton 100. The device can accommodate the natural range of motion of the human foot and ankle joints. The device can be robust enough to transmit significant force to the ground. The attachment point location can support mechanical designs within the range of motion to effectively actuate the ankle joint. The bottom sole assembly for footwear 600 can be fully integrated with the composite plate 205 and rigid heel stability component. The composite plate 205 can be integrated into footwear, with cushioning material (e.g., ethylene-vinyl acetate (EVA) foam, polyurethane (PU) foam, etc.) for comfort and cushioning. The composite plate 205 can be inserted into the sole of the shoe via a co-molding process or bonding process. The bony area around the heel and under the foot can tolerate higher force loads and more rigid materials than other areas of the foot. The amount of material, higher mass, and less compliant materials can be tolerated between the calcaneus, talus, and metatarsal joints. The most rigid and heavy materials and structures can be centered around the talus and calcaneus.

The attachment point can align the ankle joint behind the leg. To minimize rotational forces and leverage and other ergonomic opportunities, a symmetric ankle exoskeleton form factor can be used for applications such as running or jumping. A starting point for ankle exoskeletons can include the lower human attachment point around the foot, specifically a secure mounting structure below the ankle joint. For a symmetric ankle exoskeleton located behind the leg, this mounting point can allow a mechanical linkage to the upper section of the exoskeleton behind the heel. The exoskeleton can be attached to the rear of the shoe behind the heel.

This area of the shoe can include a rigid structure that limits the lateral movement of the heel and maintains alignment between the foot and the outer sole or bottom of the shoe. This structure can be combined or mechanically fastened to the composite footplate and the exoskeleton device to transfer force around the Human foot. The Human foot is generally tolerant of high force loads directly under the foot and more rigid structures around the heel. By combining the structure commonly used in footwear construction called a heel counter, which can be used to stabilize the heel over the footbed, with a composite footbed, a robust light-weight and ergonomically viable design can be achieved.

FIG. 7 illustrate a schematic diagram of a rear mounting point for the exoskeleton. A rigid structure around the heel and a composite footplate can be combined and integrated into footwear. The attachment point can be placed on the lateral heel and combine the heel counter and composite footplate into a singular component integrated directly into footwear.

The exoskeleton attachment point on the lateral heel of the foot can interface with the rest of the mechanical exoskeleton integrated into a rigid structure surrounding the heel of the foot. The device can accommodate the natural range of motion of the human foot and ankle joints. The device can be robust enough to transmit significant force to the ground. A composite structural heel counter component of the shoe or boot, which can be made with rubber or plastic, can be combined with a composite footbed. This can result in a structural, dual-purpose component can be integrated directly into the footwear. The component can optimize for low mass and high strength performance.

To minimize the exoskeleton interfering with leg movements during walking and running, an asymmetric lateral side ankle exoskeleton form factor could be used. A starting point for ankle exoskeletons can include the lower human attachment point around the foot, specifically a secure mounting structure below the ankle joint. For an ankle exoskeleton with some portion located on the lateral side of the leg on the sagittal plane, this mounting point can allow for a secure mechanical attachment point below the ankle joint.

The human foot can be tolerant of high force loads directly under the foot and more rigid structures around the heel. By combining the structure commonly used in footwear construction called the heel counter with a composite under foot plate, a robust light-weight and ergonomically viable design can be achieved. The high strength properties of engineered composites and complex curved geometry of around the heel can create areas ranging from very rigid to compliant and flexible depending on the fiber layup and thickness used in the design.

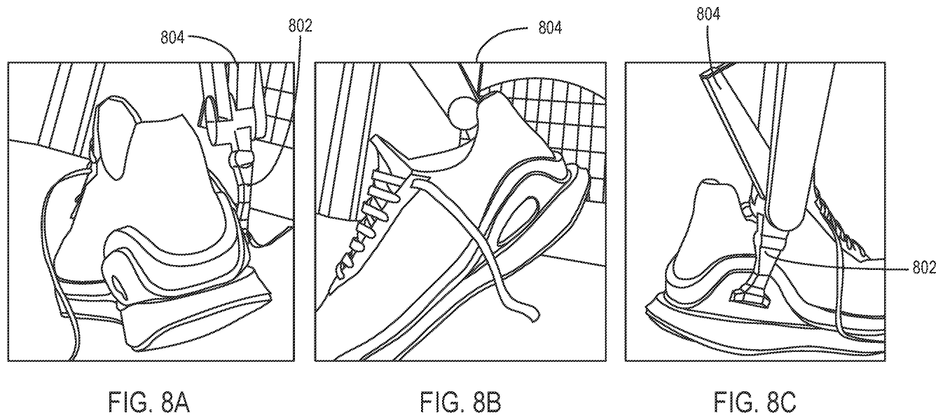

FIGS. 8A-8C illustrate a metatarsal actuator integrated into footwear sole. The metatarsal actuator integrated into footwear sole can include a carbon plate. The carbon plate can be used to transfer power to the ground from the ankle or leg exoskeleton and can be fully integrated into the footwear by combining a heel stability component with a lateral mounting tab and engineered foot plate. This can allow for reduced part count, lower mass, and increased torsional rigidity. Foam structures on both sides of the composite plate protect the foot and cushion peak force loads.

The same location around the heel of the foot can be a good mounting point to actuate the metatarsal joints. Because the bony structure of the heel of the foot is generally tolerant of rigid materials within close proximity and does not need move or flex significantly, the areas immediately surrounding the heel, known in footwear terms as the heel counter, can be used as mechanical attachment points for actuators and linkages to the foot, ankle, or both without injury or discomfort to the user.

A certain amount of the work done to move the body during walking, running and jumping is shared by the foot, ankle, leg, and hip muscle groups. In some embodiments, a foot exoskeleton could be used either in series with an ankle or leg exoskeleton, or alone to augment metatarsal joint activation and increase human performance.

To optimize for mass, comfort and robustness, these structures can be integrated directly into the footwear with low and/or high density cushioning materials such as EVA or PU foams surrounding rigid components. The rigid heel counter structure can be combined with a structural plate under the foot and from the heel area to just behind the metatarsal joint flex area. The plate can have a hinge at the metatarsal joint or terminate in a series elastic flexible actuator. The actuator could utilize a push or pull, hydraulic or pneumatic cylinder or a Bowden cable linkage.

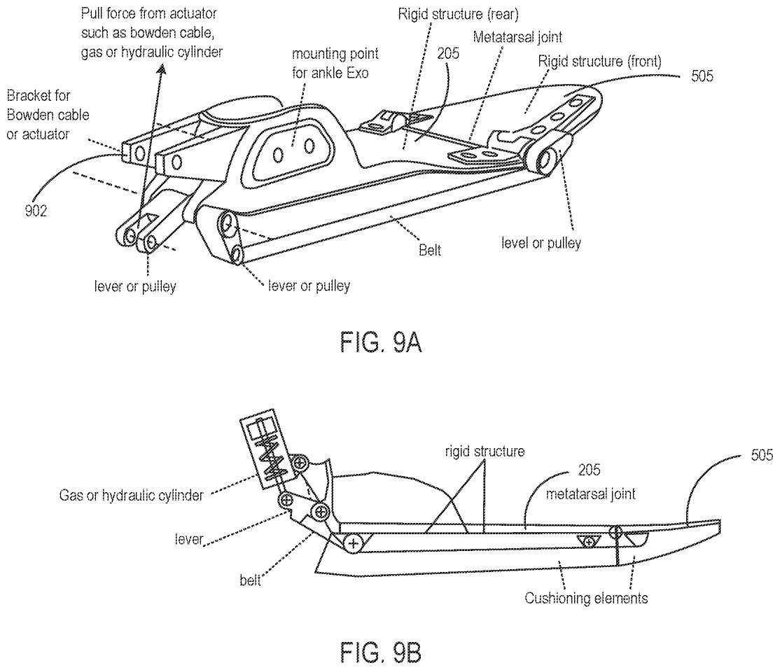

FIGS. 9A-9B illustrate the forefoot actuator (e.g., structure). The rear of the structure (e.g., the composite plate 205 or the footplate) can be suitable for mounting rigid mechanical components (e.g., mechanical component(s) 902) such as a bracket, axle, or axle supports. At least one of the sides of the footplate (e.g., proximate to the heel of the footplate) can be suitable for mounting the one or more mechanical components 902. For simplicity, the mechanical component 902 can be the bracket, which can be coupled to the attachment point (e.g., the mounting tab 620 or the mounting component). The bracket can be configured to receive force from an actuator (e.g., shown in conjunction with at least FIGS. 1, 10-12, and 43). For example, the bracket can be mounted at the first portion of the footplate. The bracket can be coupled to an actuator configured to generate force to or mechanically drive the footplate via an axis of rotation, such as relative to or about the ankle of a subject (e.g., the user of the exoskeleton 100 or wearer of the footwear 600) to augment the motion (e.g., walking, running, jumping, among others) of the subject. In this case, the force generated by the actuator can be applied to the respective portion (e.g., the first portion) of the footplate mounting the bracket that is coupled to the actuator. Hence, the force can be applied to the footplate via the axis of rotation about the ankle to augment motion. The actuator can be coupled to a lower limb of the subject wearing the footwear 600, such as below the knee of the lower limb of the subject. In various aspects, the actuator can be coupled to other parts of the subject.

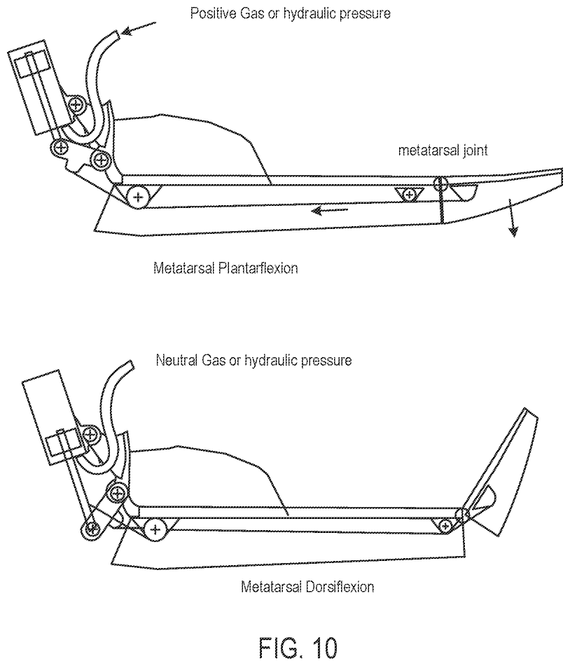

FIG. 10 illustrates the forefoot actuator. The forefoot actuator can include a piston and a cylinder. The piston and cylinder can apply tension to a belt or tensile element under the foot to actuate a hinge at the metatarsal flex area. When the piston is not activated, the belt can be slack and the foot moves freely. When the piston is pushed to the back of the cylinder, the hinged section of the plate can be drawn into plantar flexion by the tensile element rigidly affixed to the section of the plate forward of the metatarsal joints. This assembly can be integrated into the article of footwear optimizing several mechanical elements for the purposes of improving human augmentation. The mechanical actuation mechanism (e.g., mechanical hinge) can allow for a powerful activator to assist metatarsal plantar flexion

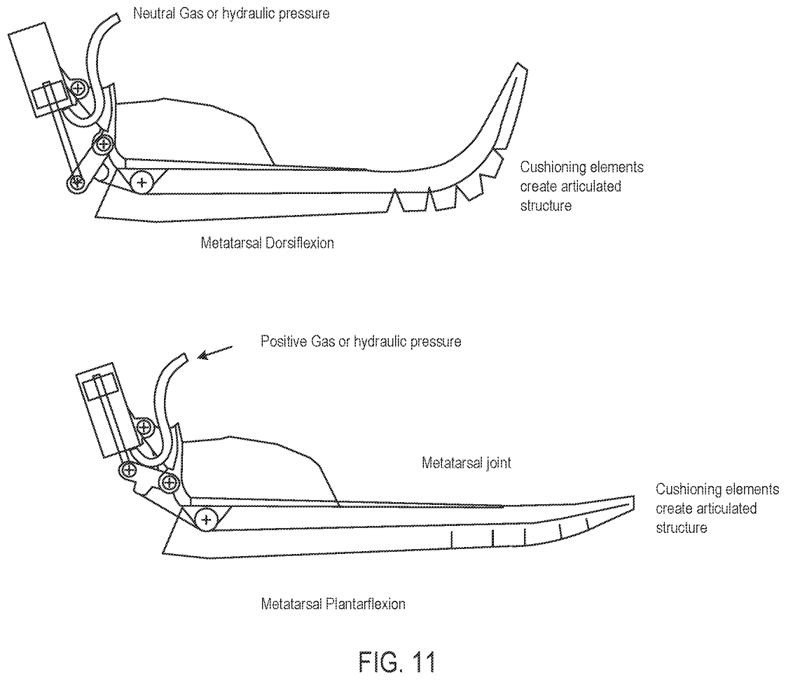

FIG. 11 illustrates the forefoot actuator. Instead of a mechanical hinge, a soft articulated actuation mechanism can be used under the forefoot metatarsal joints. This design can combine footwear components such as the bottom sole which can be manufactured from a flexible foam material to actuate the forefoot metatarsal joint. The soft articulated actuation mechanism can allow for a range of articulation and cushioning. The soft articulated actuation mechanism can allow for a powerful activator to assist metatarsal plantar flexion.

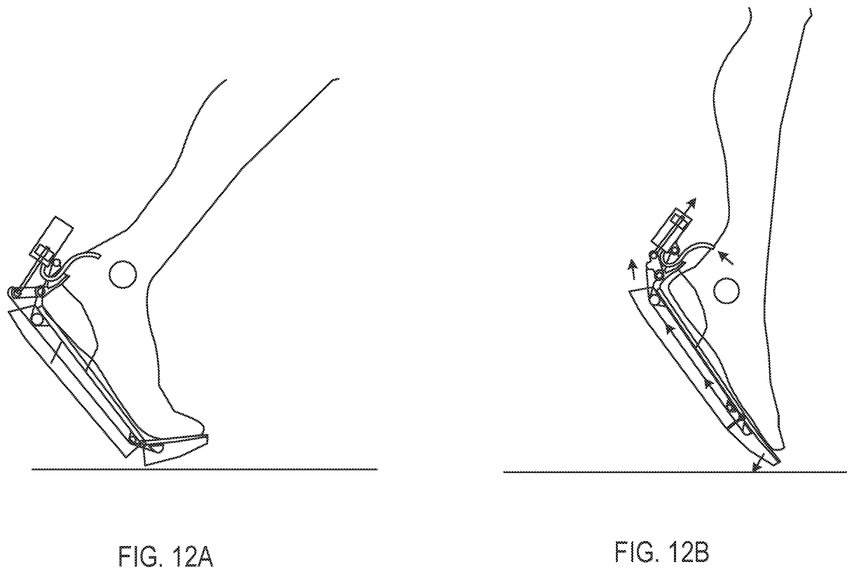

FIG. 12A illustrates a system powered in metatarsal dorsiflection. FIG. 12B illustrates a system powered in metatarsal plantarflexion. There can be mechanical opportunities to minimize mass and inertia. The motor and transmission assemblies can be some of the most massive components in the exoskeleton. Certain locations and configurations of the motor and transmission assemblies may be more advantageous than others to mitigate the effects of inertia during athletic movements. By placing the motors and transmissions in various configurations around the lower leg and foot, the effects of rotational and translational inertia can be reduced or managed in a way that is comfortable to the user and efficient for the purposes of augmenting human performance.

With each stride during walking or running, the mass of the exoskeleton attached to the lower leg can accelerate and decelerate with each step, generating multiple force loads that can be felt by the wearer. While walking, these force loads can be minimal and easily tolerated, however with athletic movements such as running, leg and foot velocity increases and these force loads can become uncomfortable. With the mass of the motor offset from the center of the leg on the lateral side, a rotational force component can be mitigated by placing the motor directly behind, or in front of the leg, aligned along the sagittal plane, bisecting the leg.

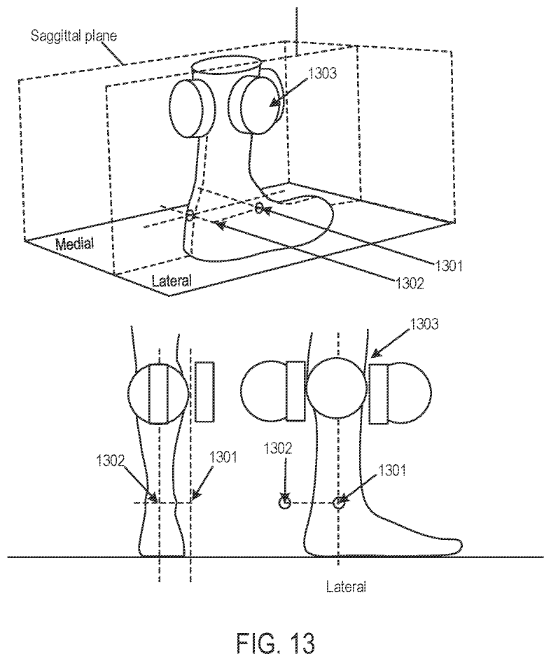

FIG. 13 illustrates a schematic diagram of the rotational axis of the ankle joint. FIG. 13 illustrates the rotational axis 1301 of the ankle joint aligned on the sagittal plane but off-set from the leg on the frontal plane, the rotational axis 1302 of the ankle joint off-set behind the ankle on the sagittal plane, but bisecting the leg on the sagittal plane, and at least one of the locations 1303 for the motor around the lower leg to manage the inertial effects of the exoskeleton mass felt during active movement.

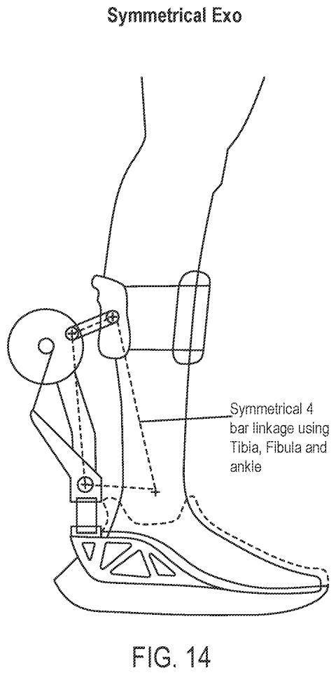

FIG. 14 illustrates a symmetrical ankle exoskeleton design. The mass and mechanical design of the exoskeleton can be packaged in a form factor directly behind or in front of the leg to manage the acceleration and deceleration of the exoskeleton mass without introducing a rotational force component. To minimize rotational forces, inertia, and to leverage other ergonomic opportunities, a symmetric ankle exoskeleton form factor could be advantageous for certain applications such as running or jumping. By aligning the ankle pivot joint on the sagittal plane bisecting the leg, behind the actual human ankle joint, a variety of linkage systems could be used. A symmetrical, behind the ankle exoskeleton design is shown in FIG. 13 with a 2 bar mechanical linkage using the lower leg and ankle to complete a 4 bar linkage when attached.

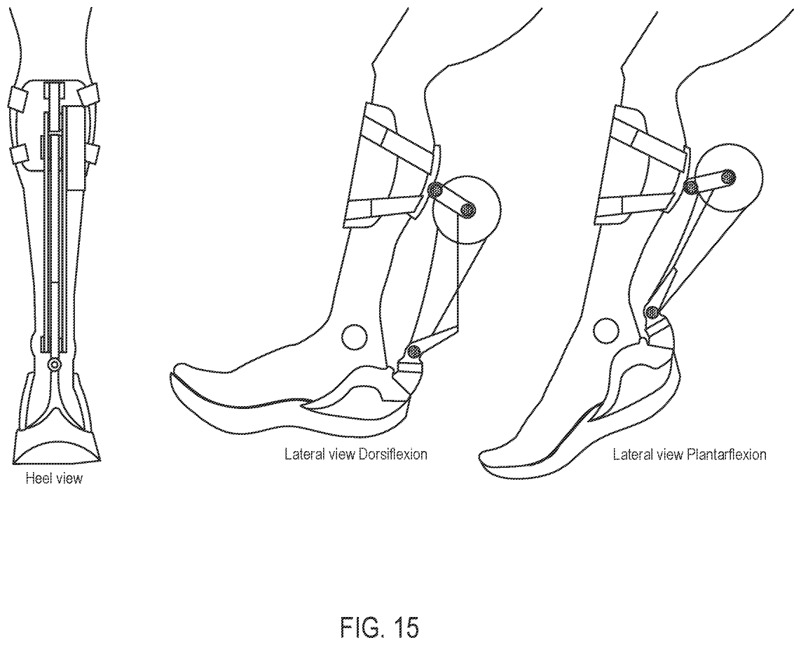

FIG. 15 illustrates a symmetrical linkage system. The symmetrical linkage system can be used to augment ankle plantar flexion.

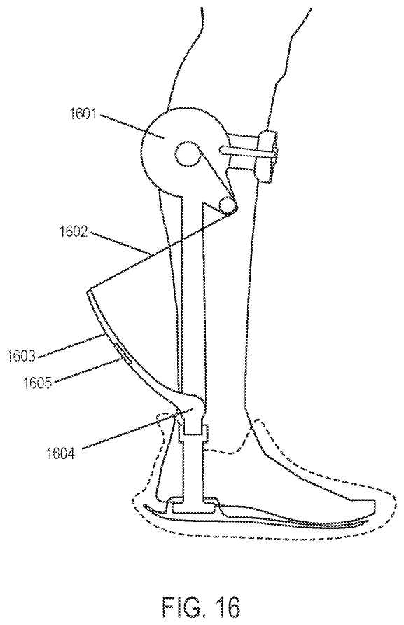

FIG. 16 illustrates an elastic ankle lever. Elastic elements can be introduced in certain components of the exoskeleton such as the ankle level, underfoot plate, or upright. The elastic elements can store and release energy during the gait cycle. The elastic element could be constructed of carbon fiber, steel, or similar material that has an elastic spring quality, allowing it to store kinetic energy when deflected and then return energy while returning to its original shape. The spring could also be implemented in a coil over rod, torsional spring, or other configuration that allows mechanical energy to be stored and released. The spring deflection can also be measured and used as an input into the control system. Storing and releasing energy during operation can increase the efficiency of the system, act as a low pass filter for torques or forces, or measure actuation forces/torques being applied. The system can include a/an motor 1601, belt 1602, elastic element 1603, ankle pivot joint 1604, and integrated strain gauge 1605. The system can include multiple motors 1601. The system can include the motor 1601 positioned at or proximate to at least one of the locations 1303. The motor 1601 can be positioned at other locations, for example, at or below the knee of the subject.

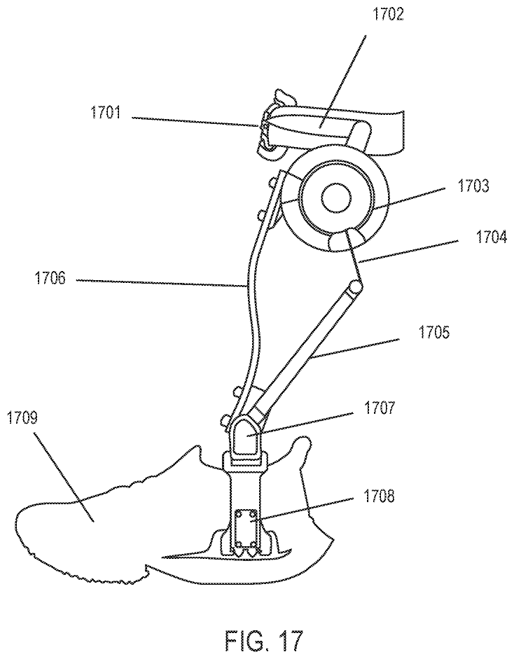

FIG. 17 illustrates an elastic exoskeleton upright element. Elastic elements can be introduced in certain components of the exoskeleton such as the ankle level, underfoot plate, or upright. The elastic elements can store and release energy during the gait cycle. The elastic element could be constructed of carbon fiber, steel, or similar material that has an elastic spring quality, allowing it to store kinetic energy when deflected and then return energy while returning to its original shape. The spring could also be implemented in a composite tube, flat leaf spring, or other configuration that allows mechanical energy to be stored and released. The spring deflection can also be measured and used as an input into the control system. Storing and releasing energy during operation can increase the efficiency of the system, act as a low pass filter for torques or forces, or measure actuation forces/torques being applied. The system can include a/an shin pad 1701, shin pad lever 1702, motor 1703 (e.g., similar to the motor 1601), belt 1704, ankle lever 1705, upright support 1706, ankle joint 1707, exoskeleton attachment point 1708, and footwear 1709 with integrated carbon plate (e.g., the footplate or the composite plate 205). The shin pad 1701 can be configured to couple to the shin of the subject below the knee of the subject. The exoskeleton attachment point 1708 can include, correspond to, or be associated with the mounting tab 620, the mounting component, or the attachment point, for example. The footwear 1709 can correspond to or include similar features or components as the footwear 600.

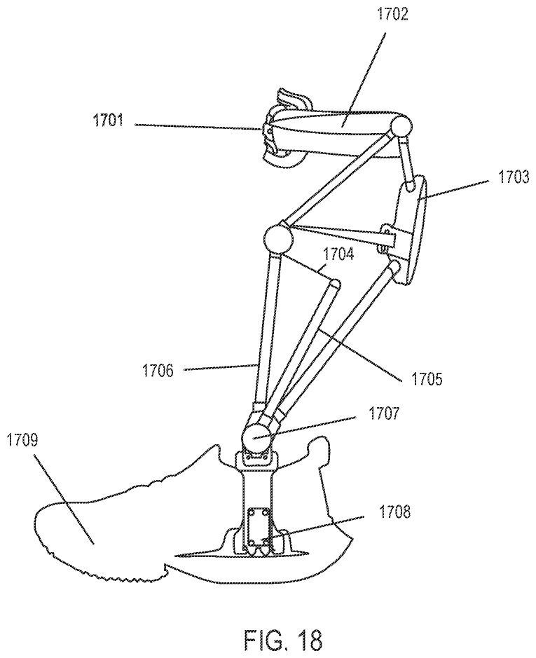

FIG. 18 illustrates a motor 1703 located behind the leg/calf. Moving the center of gravity of the system closer to the sagittal plane can reduce the inertial effects felt by the user. If the motor 1703 is placed in the sagittal plane, and the exoskeleton is connected to the lateral side of the shoe, force/torque can be routed around the leg. This can be done with a tensile element such as a cord, rope, or belt (e.g., belt 1704), and a series of pulleys. The system can include a/an shin pad 1701, shin pad lever 1702, motor 1703, belt 1704, ankle lever 1705, upright support 1706, ankle joint 1707, exoskeleton attachment point 1708, and footwear 1709.

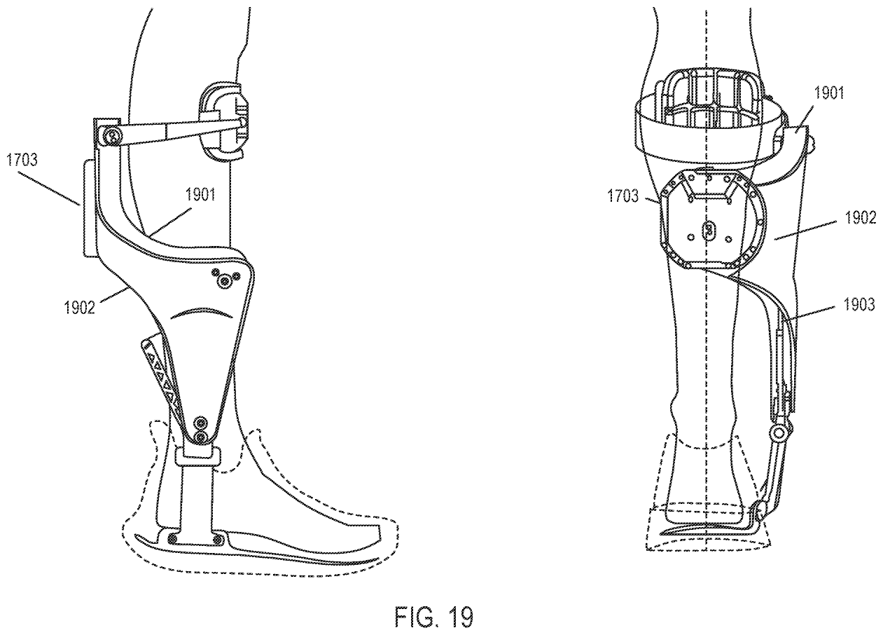

FIG. 19 illustrates a motor 1703 located behind the leg/calf. Moving the center of gravity of the system closer to the sagittal plane can reduce the inertial effects felt by the user. If the motor 1703 is placed in the sagittal plane, and the exoskeleton is connected to the lateral side of the shoe, force/torque can be routed around the leg. This can be done with a tensile element such as a cord, rope, or belt, and a series of pulleys. The system can include a/an guide pulley 1901, carbon shell 1902, motor 1703 (e.g., electric motor), and tensile element 1903. The carbon shell 1902 can be referred to as a shell or a housing for enclosing, encapsulating, or including one or more components of the exoskeleton 100. For example, the carbon shell 1902 (e.g., the housing) can include or enclose the motor 1703, the belt 1704, the ankle lever 1705, the upright support 1706, among others. The motor 1703 can be configured to generate or apply force to the footplate, such as via the ankle lever 1705 (e.g., or a linkage between the motor 1703 and the footplate). By the motor 1703 applying force to the footplate, torque can be generated about an axis of rotation of an ankle joint 1707 (e.g., ankle joint of the subject).

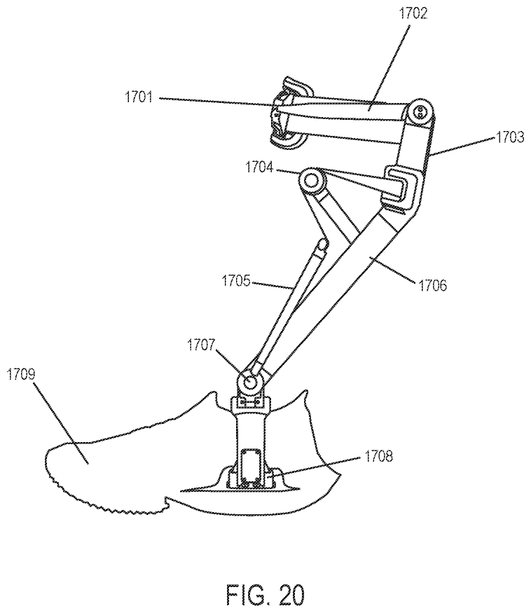

FIG. 20 illustrates a motor 1703 located behind the leg. Moving the center of gravity of the system closer to the sagittal plane can reduce the inertial effects felt by the user. If the motor 1703 is placed in the sagittal plane, and the exoskeleton is connected to the lateral side of the shoe, force/torque can be routed around the leg. This can be done with a tensile element such as a cord, rope, or belt, and a series of pulleys. Small diameter tubes can be used to create the rigid frame. The system can include a/an shin pad 1701, shin pad lever 1702, motor 1703, belt 1704, the ankle lever 1705, the upright support 1706, ankle joint 1707, exoskeleton attachment point 1708, and footwear 1709 with integrated carbon plate.

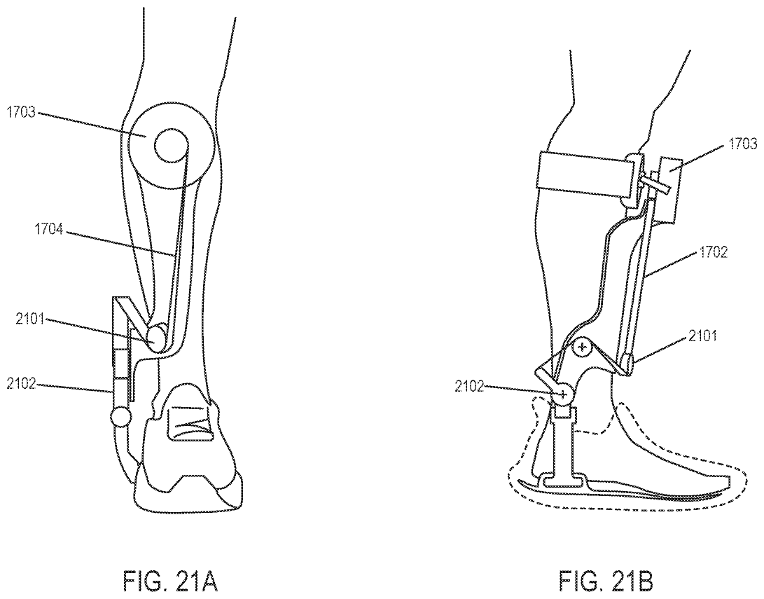

FIG. 21A-21B illustrate a motor 1703 located in front of leg/shin. Moving the center of gravity of the system closer to the sagittal plane can reduce the inertial effects felt by the user. If the motor 1703 is placed in the sagittal plane, and the exoskeleton is connected to the lateral side of the shoe, force/torque can be routed around the leg. This can be done with a tensile element such as a cord, rope, or belt, and a series of pulleys. A belt can twist and can be done in multiple small increments. The system can include a/an motor 1703, belt 1704, guide pulley 2101, and ankle pivot joint 2102.

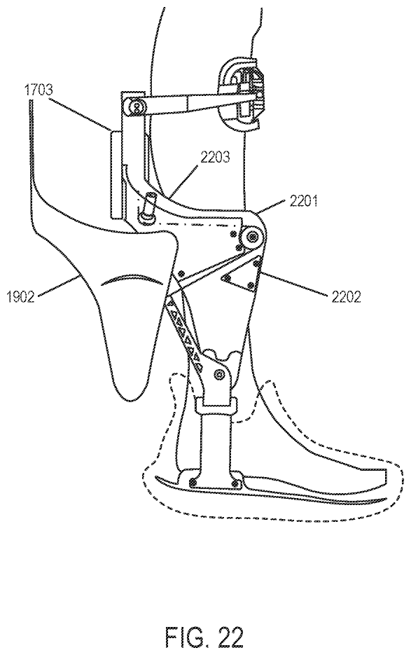

FIG. 22 illustrates a composites sheet construction. The system can include low mass, high strength materials to avoid the effects of mass and inertia during activities such as running or jumping. Carbon-fiber-reinforced polymers (CFRP) or thermo-set layup can be used for the body section. 3D printed internal structures, honeycomb or other lattice structures sandwiched between composite or other materials, or additively manufactured composite structures can be sued. The system can include a/an 3D printed structure 2201, structural carbon shell 1902 (e.g., the housing), motor 1703, 3D printed structure 2202, and 3D printed structure 2203.

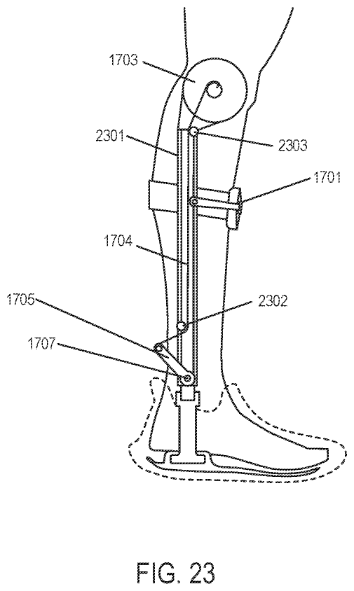

FIG. 23 illustrates an exoskeleton. The exoskeleton can include materials made from carbon tubes or extruded shapes. Low mass, high strength materials can reduce distil mass by locating motor 1703 closer to knee. Carbon tubes or extruded shapes can be exceptionally strong and rigid with low mass properties. Furthermore their internal volume can be utilized to house a belt, linkage or tensile elements to protect the wearer from moving parts within, and or protect the internal mechanism from external damage during use. The system can include a/an motor 1703, belt 1704, ankle lever 1705, ankle pivot joint 1707, composite tube 2301, shin pad 1701, guide pulley 2302, and guide pulley 2303.

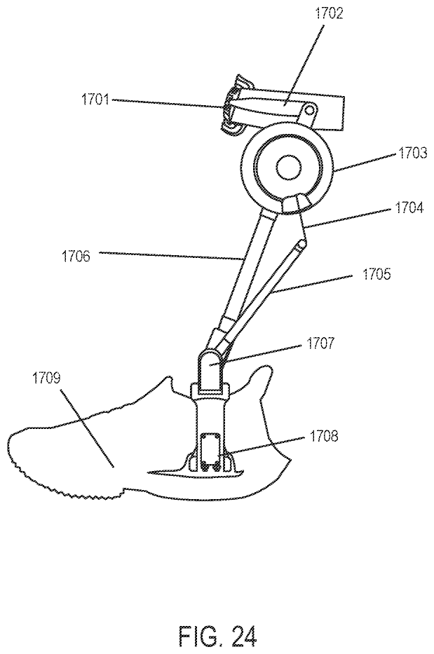

FIG. 24 illustrates an exoskeleton. The system can include a/an shin pad 1701, shin pad lever 1702, motor 1703, belt 1704, ankle lever 1705, upright support 1706, ankle joint 1707, exoskeleton attachment point 1708, and footwear 1709 with integrated carbon plate.

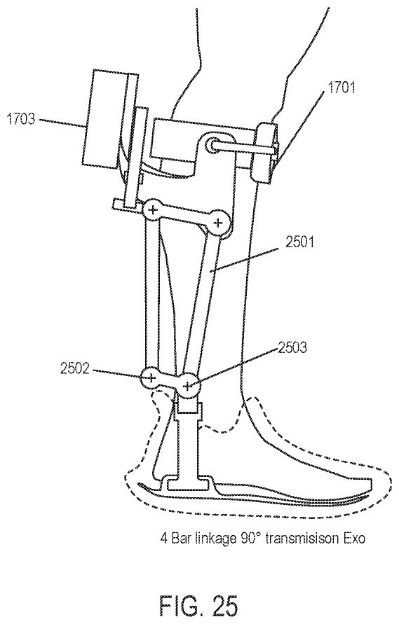

FIG. 25 illustrates an around the corner transmission 4 bar linkage. Moving the center of gravity of the system closer to the sagittal plane can reduce the inertial effects felt by the user. If the motor 1703 is placed in the sagittal plane, and the exoskeleton is connected to the lateral side of the shoe, force/torque can to be routed around the leg. Using a 4 bar linkage on the lateral side can simplify the routing path of the belt from the linkage to the motor pulley, eliminating a need to “twist” the belt or route a tensile element around a corner. The system can include a/an motor 1703, shin pad 1701, linkage 2501, linkage 2502 (e.g., ankle lever), and ankle pivot 2503. At least one of the linkages 2501, 2502 can receive the force generated by the motor 1703 to apply to the footplate (e.g., via the pivot 2503). By applying the force, the motor 1703 can generate torque about an axis of rotation of the ankle joint 1707 of the subject or at the pivot 2503.

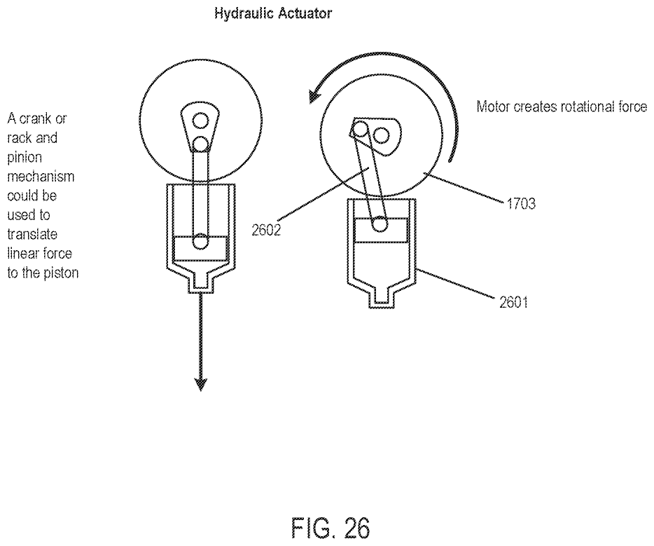

FIG. 26 illustrates a hydraulic transmission. The hydraulic transmission can include two pistons connected by a hydraulic line. If the motor (e.g., motor 1703) is placed in the sagittal plane, and the exoskeleton is connected to the lateral side of the shoe, force/torque can to be routed around the leg. A hydraulic transmission can allow the motor/actuator to be placed in any location. When force is applied from one fluidly connected piston, proportionate force can be applied to the other fluidly connected piston through a line or passage. Furthermore, a mechanical advantage can be built in to increase leverage. This could be accomplished with a Bowden cable. The system can include a/an motor 1703, piston/cylinder 2601, and crank 2602.

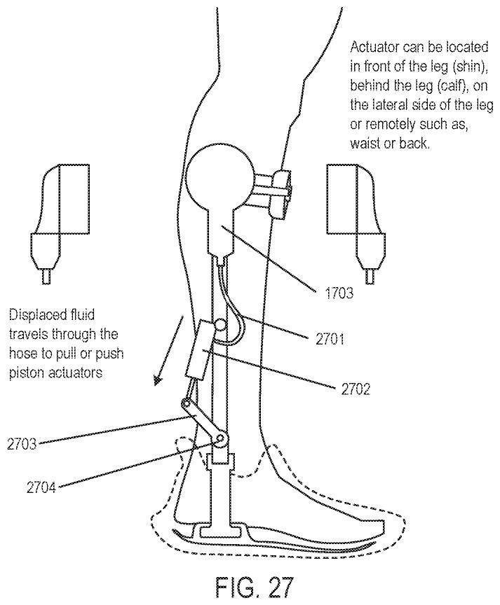

FIG. 27 illustrates a hydraulic actuator. The system can include a/an motor 1703 and hydraulic actuator cylinder/piston, hydraulic line 2701, hydraulic cylinder/piston actuator 2702, lever 2703, and ankle joint 2704.

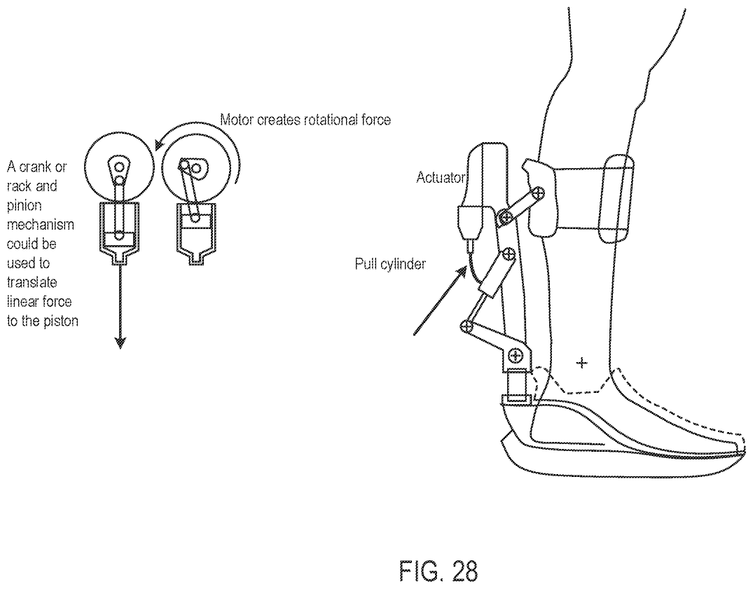

FIG. 28 illustrates a hydraulic actuator. The hydraulic actuator can be used in a symmetrical behind the heel configuration.

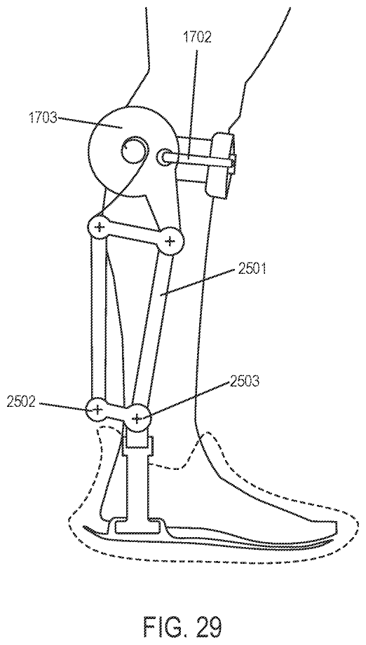

FIG. 29 illustrates a 4 bar lateral linkage. This design can allow for the use of a short belt with most of the span between the ankle and motor connected by the linkage. Added mechanical leverage can be achieved by using disproportionate lengths of top and bottom links. Mechanical force can be transferred from the upper link to the lower link in a 4 bar configuration applying torque to the ankle joint. The system can include a/an motor 1703, shin pad lever 1702, linkage 2501, linkage pivot point 2502, and ankle pivot linkage 2503.

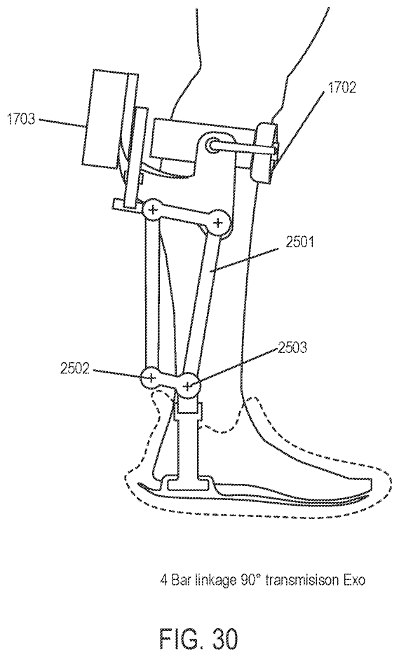

FIG. 30 illustrates a 4 bar linkage 90° transmission exoskeleton. The system can include a/an motor 1703, shin pad lever 1702, linkage 2501, linkage pivot point 2502, and ankle pivot linkage 2503.

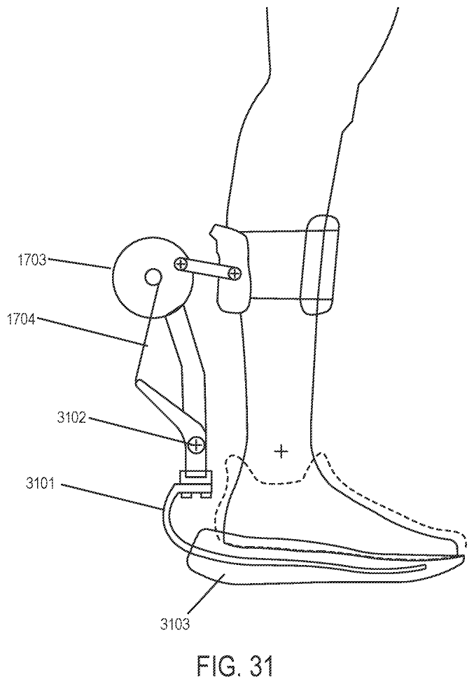

FIG. 31 illustrates a carbon fiber underfoot structure. The carbon fiber underfoot structure can be used to store and release energy elastically. Storing and releasing energy during operation can increase the efficiency of the system. A simplified version of the composite plate geometry currently used in the exoskeleton can be combined with the inverted spring designs. The system can include a/an motor 1703, underfoot spring plate 3101, ankle pivot joint 3102, belt 1704, and bottom sole 3103 of a shoe (e.g., similar to the bottom sole of the footwear 600).

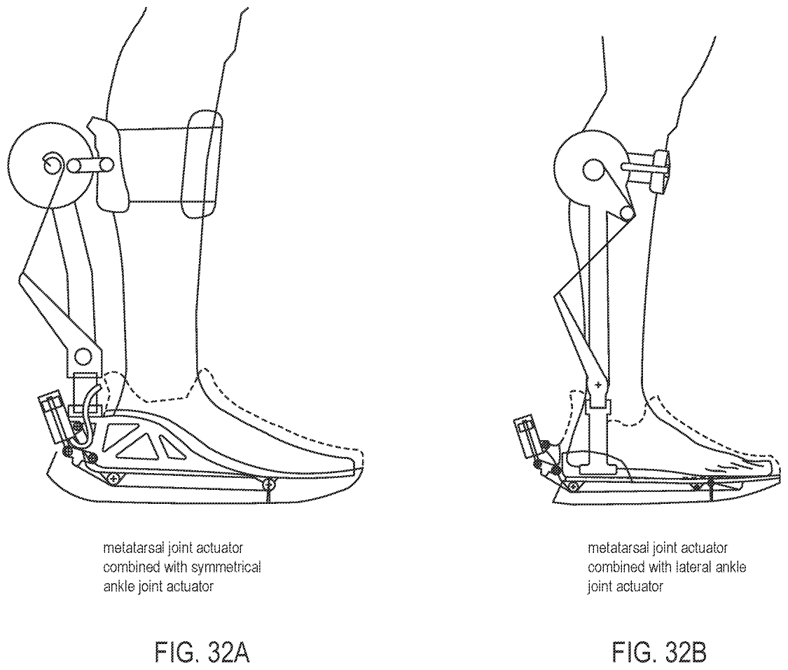

FIGS. 32A-32B illustrate a combined ankle and metatarsal actuation system. A certain amount of the work done to move the body during walking, running and jumping can be shared by the foot, ankle, leg and hip muscle groups. In some embodiments, a foot exoskeleton can be used either in series with an ankle or leg exoskeleton, or alone to augment metatarsal joint activation and increase human performance. Mechanically, ankle and metatarsal actuator systems can act independently. Control and power systems can be shared. Just as groups of muscles are timed to contract and relax in concert to move the human body, ankle and forefoot actuators, controllers and sensors can work together to augment movement by applying the appropriate consecutive forces.

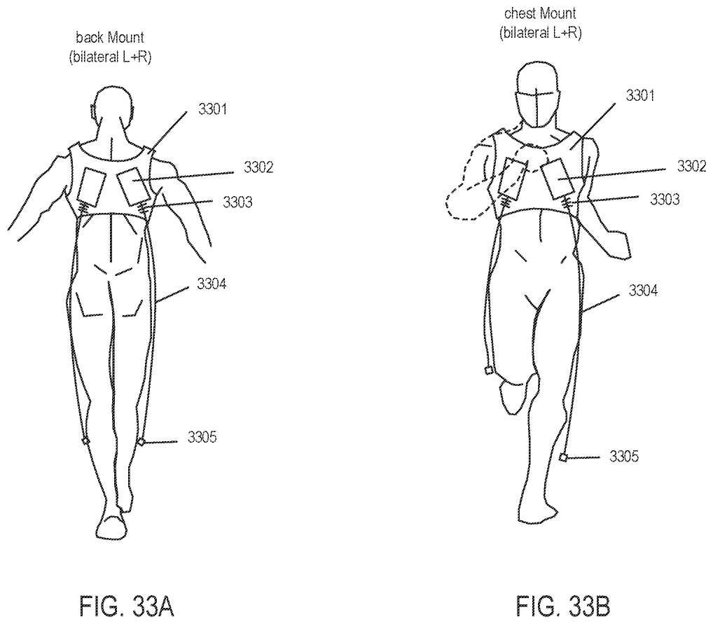

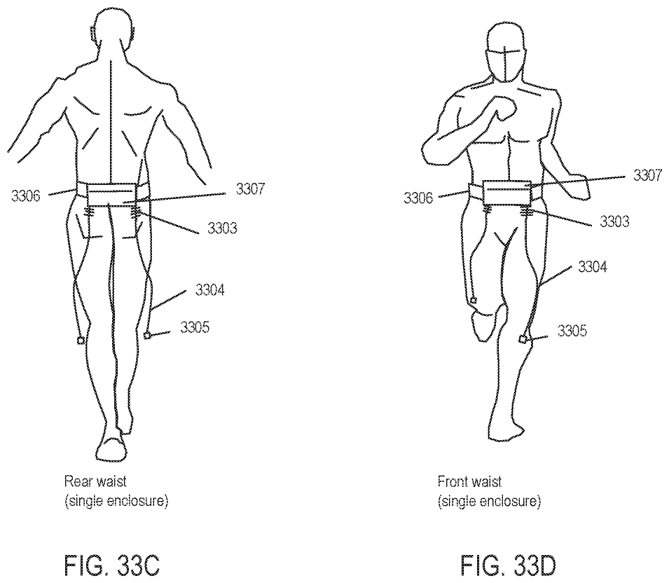

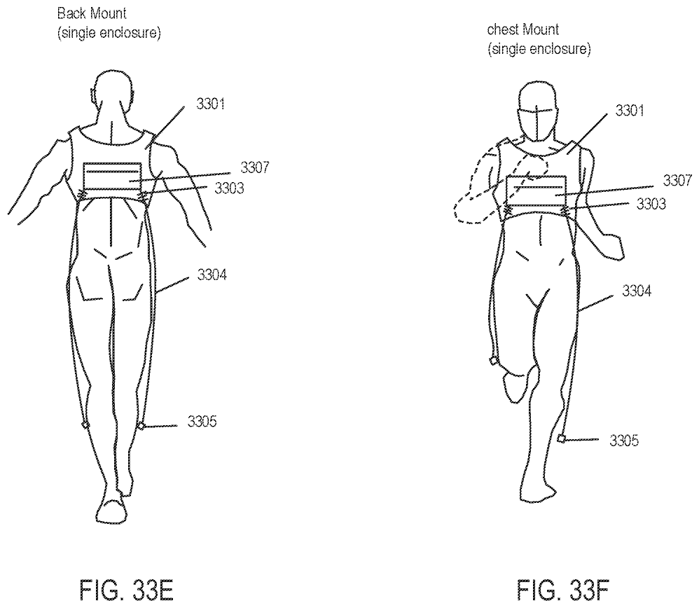

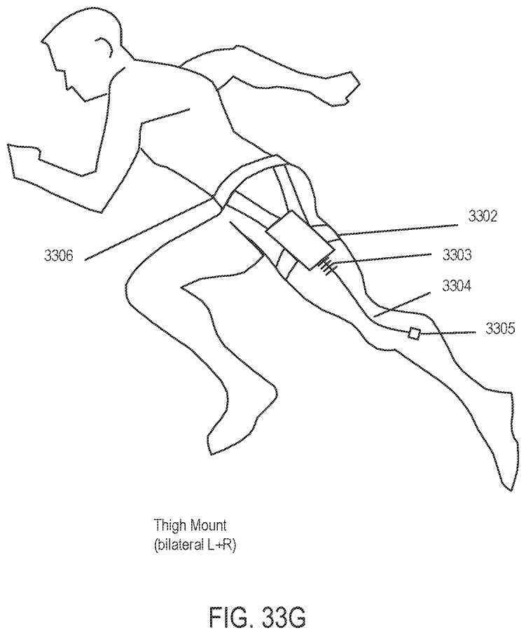

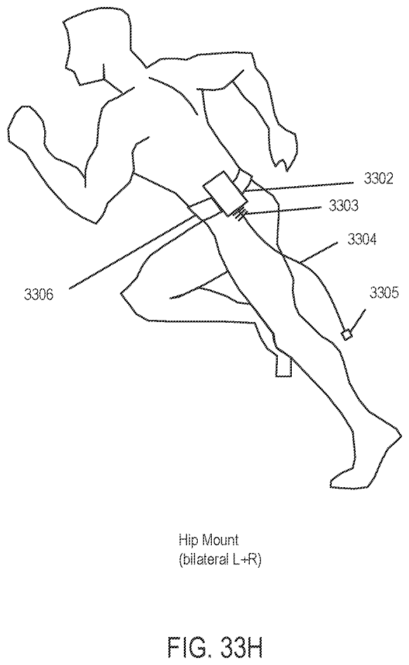

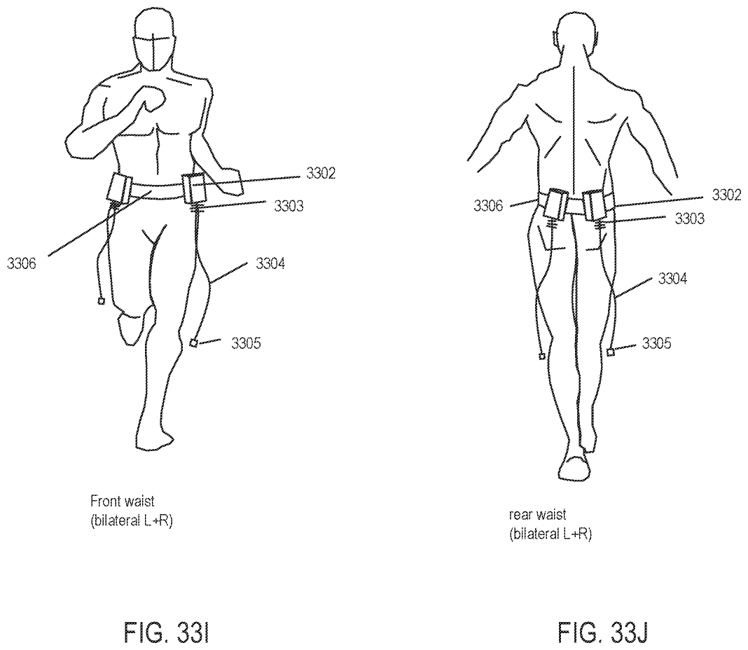

The system can include a battery attachment as shown in FIGS. 33A-33J. The system can reduce distil mass and discomfort fatigue associated with carrying mass on the body during rigorous athletic activities such as running and jumping. The battery can be positioned at various locations around the body. For example battery can be placed closer to the center of mass.

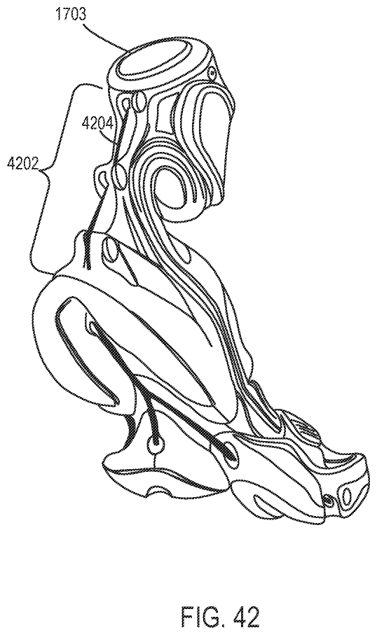

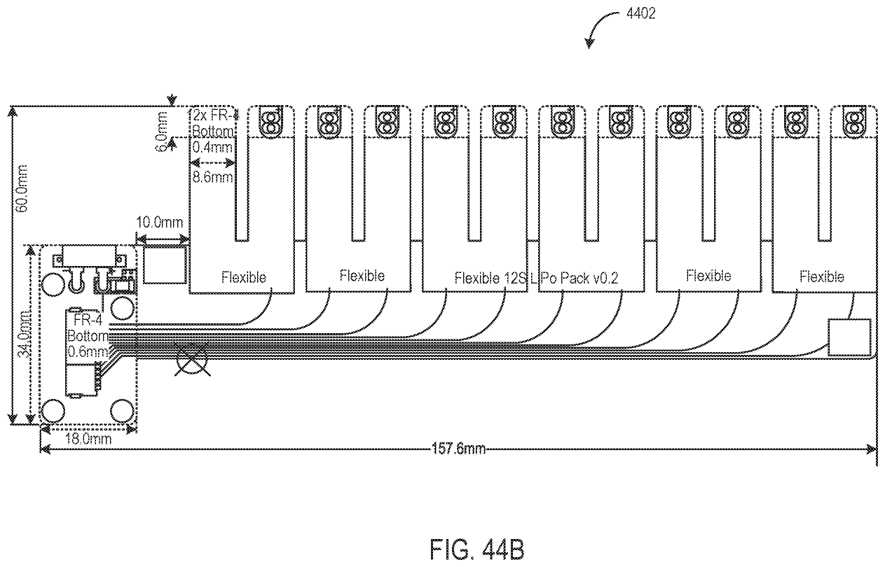

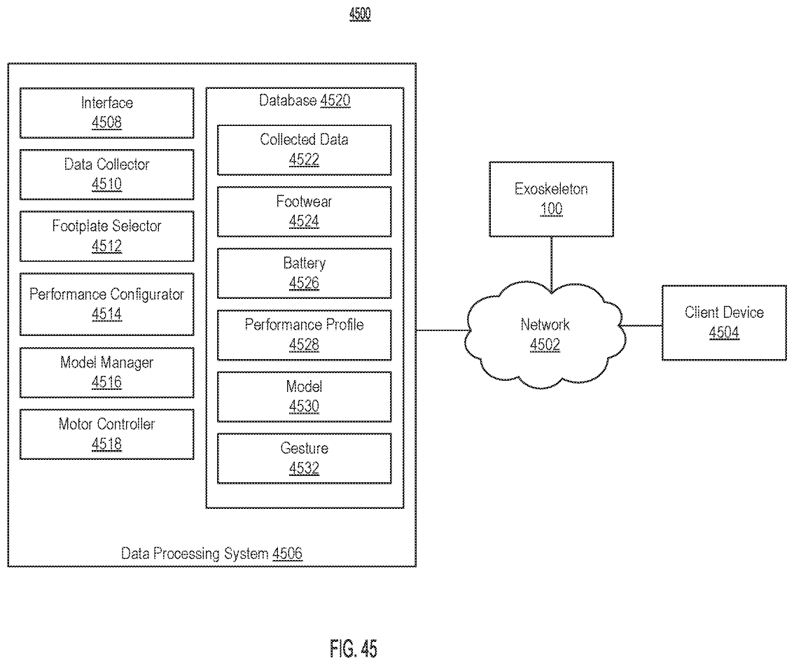

FIG. 33A illustrates a back mount battery attachment. The back mount battery attachment can include a bilateral L+R back mount battery attachment. The system can include a/an vest 3301, harness or sports bra, battery module 3302, cable strain relief 3303, cable 3304 (e.g., wire) to the exoskeleton (e.g., exoskeleton 100), and plug 3305 (e.g., connector or interface) to the exoskeleton. The battery module 3302 can include, correspond to, or be referred to as a battery pack (e.g., battery pack 3302). The battery module 3302 can be included in a container, for example. The battery module 3302 can include one or more battery cells, such as discussed in further detail in at least FIG. 42 or 44.

For example, the battery module 3302 can be integrated in or as part of the shin pad 1701, vest 3301, belt, 3306, among other components or structures. The battery module 3302 can be or include flexible battery. The battery module 3302 can be configured to couple to a calf behind the shin of the subject, among other body parts of the subject. The battery module 3302 can include a printed circuit board (PCB) or a substrate upon which the circuitry of the battery module 3302 is formed, which can be flexible with the battery module 3302 or the curvature of one or more body parts of the subject. The PCB can be configured to connect individual battery cells in series (or in some cases, in parallel). The battery module 3302 can include a mechanical structure with flexible members (or components). The flexible members can allow or enable the battery module 3302 to flex or bend along various curved radii (e.g., curvature of the body parts or of the exoskeleton component(s)). The battery module 3302 can include one or more connectors for connection to various components to enable the functionality of the exoskeleton 100. For example, the battery module 3302 (e.g., battery pack) can include a first connector (e.g., at least one of the cable 3304 or plug 3305) for electrical connection between the battery module 3302 to the motor 1703. The battery module 3302 can include a second connector (e.g., another cable 3304 or plug 3305) configured to electrically connect to a charger (not shown) for charging the battery module 3302 (e.g., the battery pack or one or more battery cells). In some cases, the second connector can correspond to a charging port. The second connector can allow the charger to directly power the exoskeleton 100 (e.g., the motor 1703 of the exoskeleton 100), for example.

FIG. 33B illustrates a chest mount battery attachment. The chest mount battery attachment can include a bilateral L+R chest mount battery attachment. The system can include a/an vest 3301, harness or sports bra, battery module 3302, cable strain relief 3303, cable 3304 to exoskeleton, and plug 3305 to exoskeleton.

FIG. 33C illustrates a rear waist battery attachment. The rear waist battery attachment can include a single enclosure rear waist battery attachment. The system can include a/an belt 3306, pouch 3307 (or container) holding L+R battery modules (e.g., similar to the battery module 3302 or the battery pack), cable strain relief 3303, cable 3304 to exoskeleton, and plug 3305 to exoskeleton.

FIG. 33D illustrates a front waist battery attachment. The front waist battery attachment can include a single enclosure front waist battery attachment. The system can include a/an belt 3306, pouch 3307 or container holding L+R battery modules, cable strain relief 3303, cable 3304 to exoskeleton, and plug 3305 to exoskeleton.

FIG. 33E illustrates a back mount battery attachment. The back mount battery attachment can include a single enclosure back mount battery attachment. The system can include a/an vest 3301, harness or sports bra, pouch 3307 or container holding L+R battery modules, cable strain relief 3303, cable 3304 to exoskeleton, and plug 3305 to exoskeleton.

FIG. 33F illustrates a chest mount battery attachment. The chest mount battery attachment can include a single enclosure chest mount battery attachment. The system can include a/an vest 3301, harness or sports bra, pouch 3307 or container holding L+R battery modules, cable strain relief 3303, cable 3304 to exoskeleton, and plug 3305 to exoskeleton.

FIG. 33G illustrates a thigh mount battery attachment. The thigh mount battery attachment can include a bilateral L+R thigh mount battery attachment. The system can include a/an belt 3306 and thigh harness, battery module 3302, cable strain relief 3303, cable 3304 to exoskeleton, and plug 3305 to exoskeleton.

FIG. 33H illustrates a hip mount battery attachment. The hip mount battery attachment can include a bilateral L+R hip mount battery attachment. The system can include a/an belt 3306, battery module 3302, cable strain relief 3303, cable 3304 to exoskeleton, and plug 3305 to exoskeleton.

FIG. 33I illustrates a front waist battery attachment. The front waist battery attachment can include a bilateral L+R front waist battery attachment. The system can include a/an belt 3306, battery module(s) 3302, cable strain relief 3303, cable 3304 to exoskeleton, and plug 3305 to exoskeleton.

FIG. 33J illustrates a rear waist battery attachment. The rear waist battery attachment can include a bilateral L+R rear waist battery attachment. The system can include a/an belt 3306, battery module(s) 3302, cable strain relief 3303, cable 3304 to exoskeleton, and plug 3305 to exoskeleton.

In some cases, the battery module 3302 can include or correspond to a single battery pack. In some other cases, the battery module 3302 can include or correspond to multiple battery packs. The one or more battery packs can be equipped or replaced by the subject. The battery pack can include at least a type of battery cell. The type of battery cell can indicate at least one of a capacity of the battery or a maximum peak current (e.g., or voltage or power) of the battery. The capacity of the maximum peak current of the battery cells associated with the battery pack can be used to optimize the lifetime of the battery or the energy (e.g., performance) of the motor 1703.

For example, based on at least the type of battery cell (e.g., the capacity or maximum peak current), a performance profile can be selected to optimize the lifetime (e.g., setting a threshold energy output below the maximum peak current or lowering the energy output at a predetermined remaining capacity) or optimize the energy output (e.g., setting the threshold energy output to the maximum peak current or maintain the energy output at any capacity). In some cases, the battery pack (e.g., a first battery pack) can be replaced with another battery pack (e.g., a second battery pack) with a greater number of battery cells (e.g., of similar type or different types). With the greater number of battery cells, the second battery pack can include at least one of a relatively greater capacity or maximum peak current compared to the first battery pack, for example. In this case, the performance profile (e.g., a second performance profile) associated with the second battery pack can include a higher threshold energy output relative to the performance profile (e.g., a first performance profile) of the first battery pack. Further, the first battery pack may have less lifetime (e.g., or be rated with less remaining operating time) compared to the second battery pack. The various thresholds discussed herein can be configured or predetermined by the administrator of the exoskeleton 100, the controller, or the data processing system, for example.

In various aspects, the exoskeleton 100 can include multiple motors (e.g., a first motor and a second motor). For example, the first motor can be used to drive or apply force to a first footplate (e.g., one of the footwear 600 of the subject) and the second motor can be used to apply force to a second footplate (e.g., the other footwear 600 of the subject). The first motor can be electrically connected to a first battery pack. The second motor can be electrically connected to a second battery pack. The first and second battery packs may include similar or different types of battery cells. The state of charge of the first battery pack can be balanced with the state of charge of the second battery pack (e.g., the state of charge controlled by a controller or a data processing system described in conjunction with FIG. 45). In some cases, the energy output of the first and second battery packs can be balanced. In some other cases, the state of charge or the energy output between the first and second battery packs may be different.

In some cases, the motor 1703 can be connected to multiple battery packs. The multiple battery packs can include similar or different types of battery cells (or different performance, such as different levels of energy output or capacities). Based on the performance or efficiency of the respective battery packs, the controller (or the data processing system as described in conjunction with FIG. 45) can select at least one of the battery packs associated with a performance profile (or select a performance profile associated with one of the battery packs) to power to the motor 1703 in accordance with the performance profile. By delivering power to the motor 1703, the motor 1703 can augment the activity of the subject, such as facilitating movement or assisting with performing the movement.

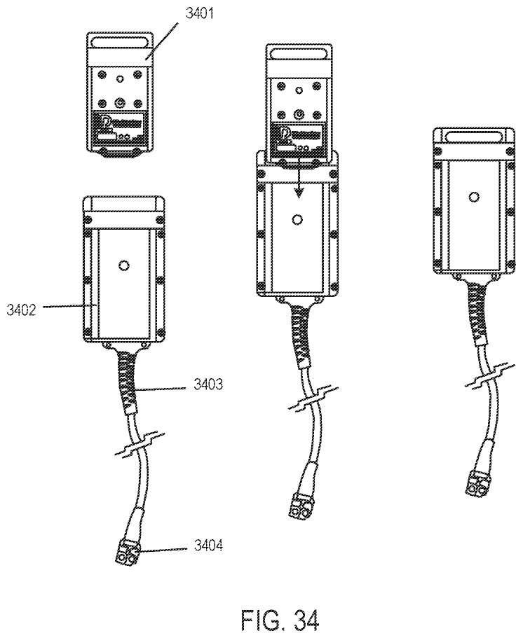

FIG. 34 illustrates a battery module. The system can include a/an BA30 battery 3401 (e.g., similar to the battery module 3302 or the battery pack), battery receptacle 3402 (e.g., sometimes referred to as a battery container, holder, or pouch, similar to the pouch 3307), cable strain relief 3403 (e.g., similar to the cable strain relief 3303), and power plug 3404 (e.g., similar to the plug 3305) to exoskeleton (e.g., exoskeleton 100).

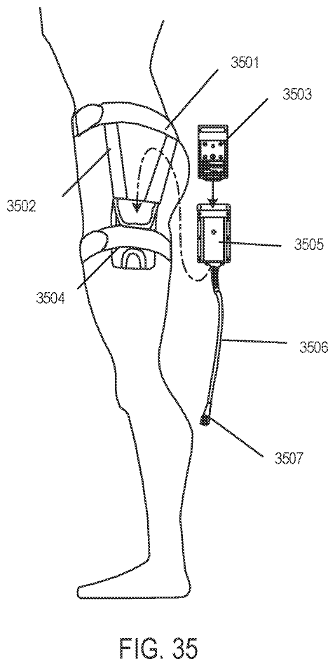

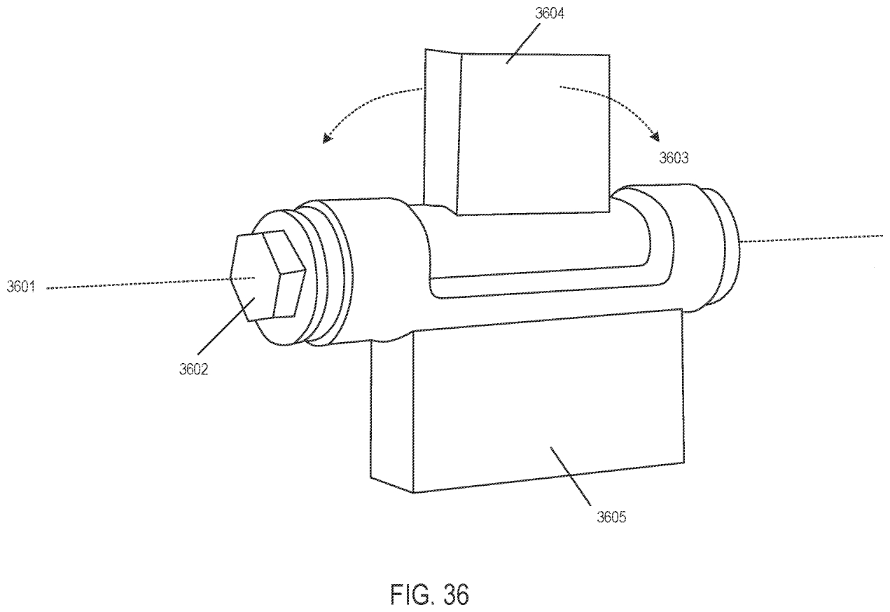

FIG. 35 illustrates a battery module and attachment. The system can include a/an waist belt 3501, thigh harness 3502, BA30 battery 3503 (e.g., similar to battery 3401), soft pouch to hold receptacle 3504, battery receptacle 3505, cable 3506 to exoskeleton, and plug 3507 to exoskeleton.

FIG. 36 illustrates a centering mechanism. The centering mechanism can be used on the shin lever, shin guard, or eversion/inversion joints. The centering mechanism can be used to reduce torque due to self-weight. The centering mechanism can help keep alignment of the system during use. The centering mechanism can provide for easier donning/doffing compared to without the centering mechanism. The centering mechanism can include sticky hinges. The system can include a/an rotational axis 3601 of hinge, threaded tensioner 3602, joint freedom 3603, top part 3604 of hinge, and bottom part 3605 of hinge.

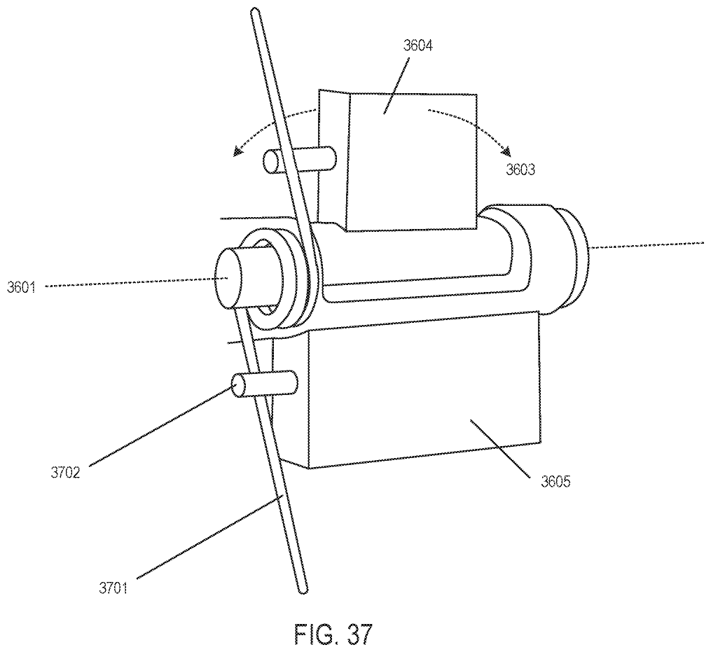

FIG. 37 illustrates a centering mechanism. The centering mechanism can be used on the shin lever, shin guard, or eversion/inversion joints. The centering mechanism can be used to reduce torque due to self-weight. The centering mechanism can help keep alignment of the system during use. The centering mechanism can provide for easier donning/doffing compared to without the centering mechanism. The centering mechanism can include a torsion spring. The system can include a/an hinge axis of rotational 3601, torsion spring 3701, joint freedom 3603, top part 3604 of hinge, bottom part 3605 of hinge, and spring stop 3702.

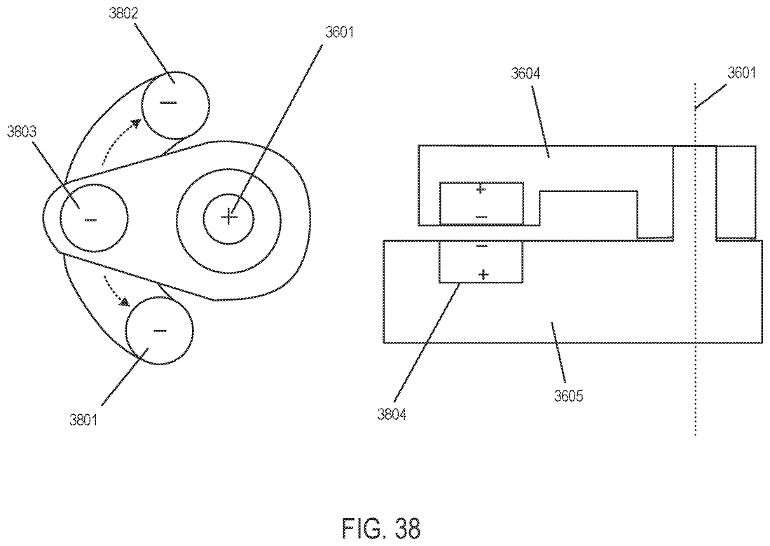

FIG. 38 illustrates a centering mechanism. The centering mechanism can be used on the shin lever, shin guard, or eversion/inversion joints. The centering mechanism can be used to reduce torque due to self-weight. The centering mechanism can help keep alignment of the system during use. The centering mechanism can provide for easier donning/doffing compared to without the centering mechanism. The centering mechanism can include one or more magnets. The system can include a/an hinge axis of rotational 3601, top part 3604 of hinge, bottom part 3605 of hinge, stationary magnet 3801 at end of travel, stationary magnet 3802 at end of travel, moving magnet 3803, and polarity of magnets 3804 oppose each other.

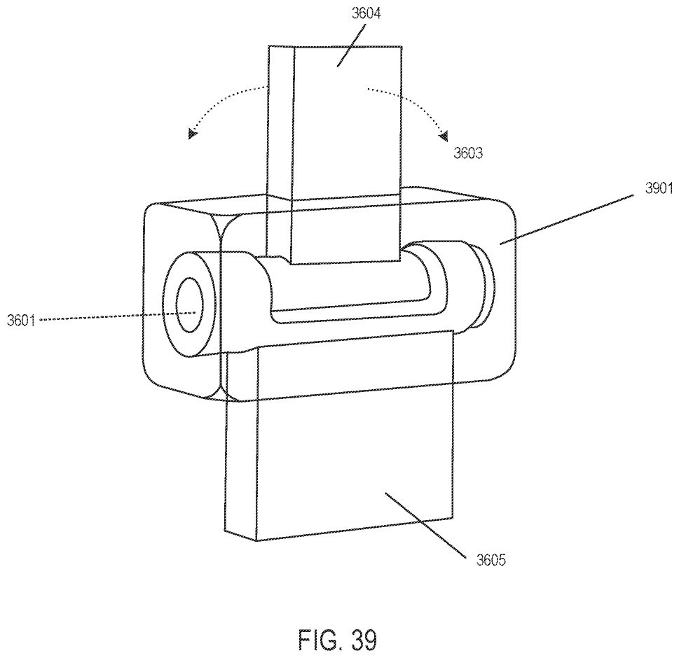

FIG. 39 illustrates a centering mechanism. The centering mechanism can be used on the shin lever, shin guard, or eversion/inversion joints. The centering mechanism can be used to reduce torque due to self-weight. The centering mechanism can help keep alignment of the system during use. The centering mechanism can provide for easier donning/doffing compared to without the centering mechanism. The centering mechanism can include an elastomeric material encapsulating joint. The system can include a/an axis of rotational 3601, elastomeric material encapsulating joint 3901, joint freedom 3603, top part 3604 of hinge, and bottom part 3605 of hinge.

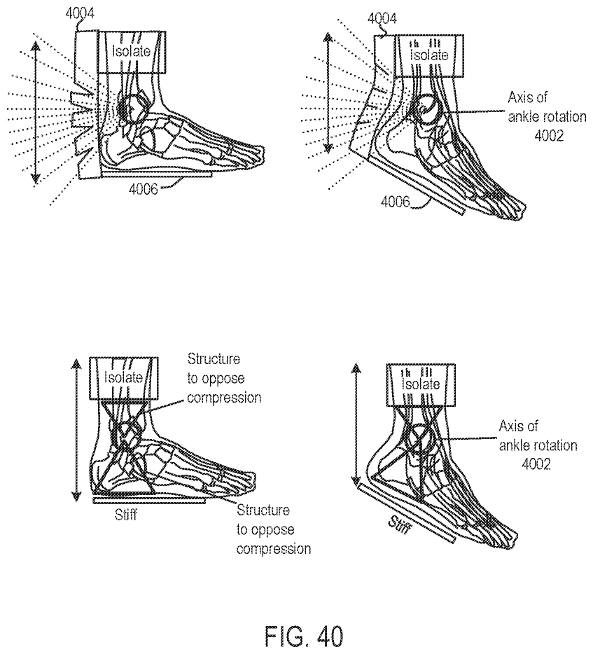

FIG. 40 illustrates a bionic shoe soft actuator. The bionic shoe soft actuator can augment walking in a familiar form-factor without introducing hard, heavy materials. The bionic shoe soft actuator can include soft actuators 4004, and rigid and compliant structures 4006. The actuator can provide torque or force about an axis of ankle rotation, as depicted in FIG. 40.

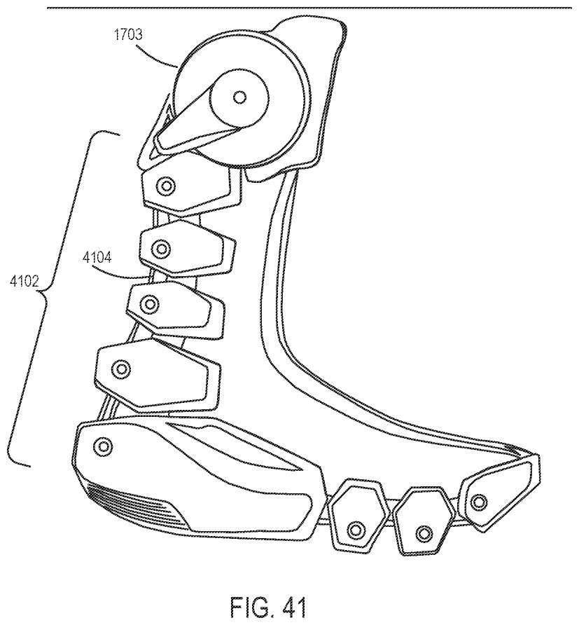

FIG. 41 illustrates a bionic shoe. The bionic shoe can augment walking using soft and compliant structures. The underfoot cushioning, and forefoot and heel actuation can be accomplished in the same assembly. A motor (e.g., motor 1703), pulley, and/or belt 4104 can draw a series of foam blocks 4102 under tension to create a rigid structure in the ankle and foot plantar flexion position. When the belt is slack, the foot can move freely without resistance.

FIG. 42 illustrates a bionic shoe. The bionic shoe can augment walking using soft and compliant structures. The underfoot cushioning, and forefoot and heel actuation can be accomplished in the same assembly. A motor, pulley, and/or belt 4204 can draw a tensile element 4202 to create a rigid structure in the ankle and foot plantar flexion position. When the belt is slack, the foot can move freely without resistance.

The system can detect activity based on inertial measurement unit (IMU) and/or power consumption. A controller can infer what the user is doing with the exoskeleton. With this information, the controller can be updated. The information can include information about road conditions (e.g., trail vs. road) based on sensor data (IMU, power profiles). The information can include a ratio of peak vs. average motor current. The information can include consistency of the gait frequency.

The system can include electronics and software. The system can include voltage boost and pack type detection. For example, the system can use a switch-mode power supply (SMPS) (e.g., a power supply with a switching regulator to convert electrical power efficiency) to increase the bus voltage for high-speed parts of the gait. The BMS can report the cell capacity, maximum peak current, etc. Different packs can be used with a given exoskeleton. Some exoskeletons can favor energy density over power density.

The system can use a SMPS as a way to accommodate different power profiles, and battery types. Boosting the voltage can allow the controller to behave more consistently even if the battery is of a different type.

Batteries can be built with a specific purpose. For example, using the same cells, one can build a pack (e.g., battery pack) that can have a longer lifetime (e.g., cells are not discharged below 20%, lower peak limits) or a higher power/energy output (e.g., discharge down to 10%, allow more peaks). The BMS can allow the first pack to do 500 cycles while the second pack may be limited to 200 cycles for safety reasons.

The system can include a BMS with activity and gait phase dependent protections and limits. The BMS and main controller can exchange data. The main controller can change the max motor current based on battery voltage. The main controller can request a change to the safety limits. For example, if a user is walking, the system can use 2 A. If the user is running, the system can allow 20 A for the next 100 ms. In emergency mode, the system can allow discharging below limits, the brick the pack.

The system can include a local battery with range extender. For example, the system can include a large battery located at the waist or anywhere else to optimize for energy density. The system can include a small battery located on the exoskeleton to optimize for power density. The system can include a large battery which can trickle charge a small battery. The system can include a small battery which handles large current pulses. The system can include a small battery which may have a much shorter lifespan than large battery. The system can include a small battery which can be replaced by super capacitors.

The activity of the system can be controlled via an application (e.g., app). For most applications, the exoskeleton controller can automatically detect the activity and augment it, but for certain applications, the user can force the controller into a specific mode. For example, the use can put the exoskeleton into “sprint mode”. This could eliminate “wasting” 3-4 steps before augmenting. The system can put the exoskeleton into a trail-running mode will support very dynamic events, while a road running mode may choose to filter out more transients to provide a smoother experience. If the user informs the controller that they are about to go downhill for a while, a special regeneration mode may be triggered.

The system can include activity-based embedded system processing and battery usage. Based on the dynamic aspect of the activity, the system can sample and compute at a different rate. For trail running where a lot of things change gait by gait, the system can refresh very quickly. For slow walking, the system can sample slower, thus conserving battery energy. The system can have a low power mode for the observation phase. Until sensors detect event X, the system can run the CPU slowly. When the event is detected, the system can increase the clock frequency to support real-time math.

The system can enable more power when racing a segment. If Garmin Connect, Strava or an equivalent app detects a starred segment, the system can unlock more power to help the user achieve a personal record.

The system can include an augmented sport tracking category. The system can add an “e-shoe” category to other applications. The system can subtract exoskeleton power from calorie estimate to correctly represent the amount of calories consumed by the athlete (e.g., user). The system can include a human-machine efficiency metric.

The system can be structured as a capacitor. The composite materials can be built in layers, akin to the construction of capacitors. Interleaving conductive film in the carbon fiber layup process can create large capacitors. These capacitors can be used as an energy reserve. The armature and/or the struts can be designed to store mechanical energy as well as electrical energy.

The system can include a hand-powered battery charger. The exoskeleton can have a hand crank. The exoskeleton can be put into hand crank mode. The controller can provide resistance, and turning of the hand crank can charge that battery. The battery can be used to power a phone or flashlight.

The system can include battery charging a battery. Instead of augmenting one leg and not the other or having one exoskeleton user with 90% batteries and one with 20% batteries who moves at the speed of the slowest, the system can allow batteries to be connected to one another. The firmware can detect a second battery and enter a charging mode where both batteries can reach an equilibrium point.

The system can include smart shoe laces. The system can adjust the tightness of the shoe laces based on activity (e.g., running, walking, sitting, jumping, amount of load being carried by the user). Pressure can be released (e.g., by 10%) for some phases of the gait. The exoskeleton battery can be used to power the shoe laces. The system can include light-up shoes. A logo can light up based on user activity, power delivered, or sensor activity.

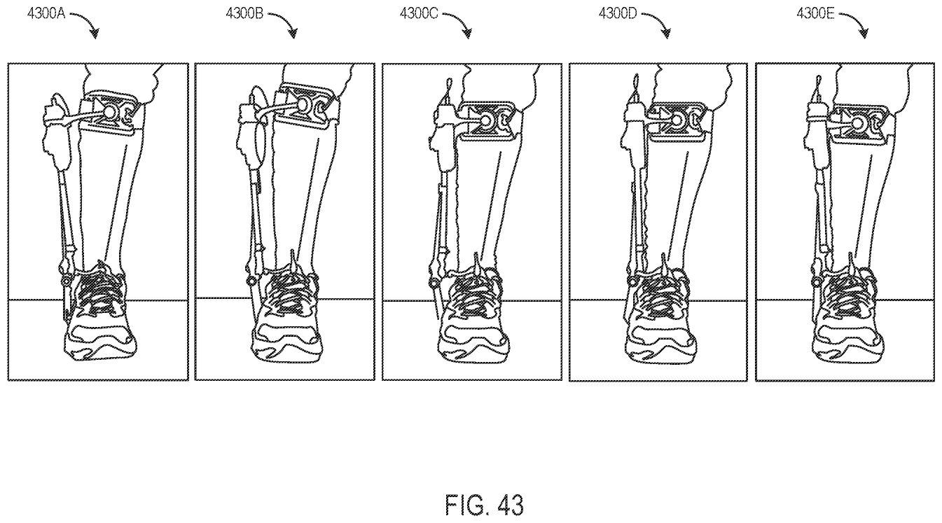

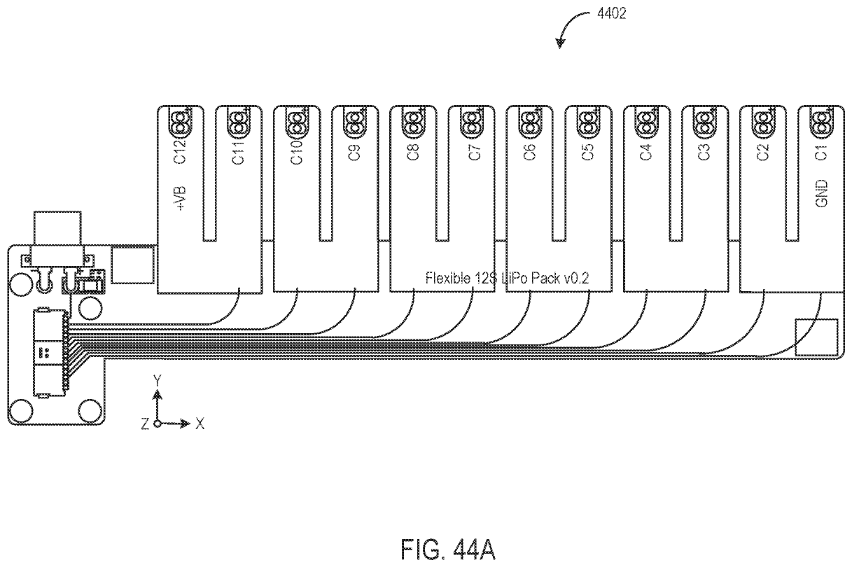

FIG. 43 illustrates example configurations 4300A-E of shin levers (schlevers) and quick-disconnects (QDs). A system can modify a commercial off-the-shelf (COTS) shoe for compatibility or to be compatible with an exoskeleton. The COTS footwear (e.g., using shoes as an example herein) can be any shoe available in-store, online, or other entities selectable or purchasable by the user. The system can determine whether the shoe is compatible (e.g., can be modified to be compatible) with the exoskeleton based on the sole specification (e.g., thickness, drop, etc.) of the shoe. In some cases, the system can select one or more shoes for compatibility with the exoskeleton. The system can use a tool or device (e.g., a saw with a type of jig or other cutting instruments) to split the mid-sole of the shoe into at least two parts. In some cases, at least a portion (e.g., area, bridge, subsection, layer, etc.) of the mid-sole can remain connected during the splitting of other portions.

The system can select an insert (e.g., footplate, insole, etc.) for the shoe based on at least one of the size of the shoe, a type of application (e.g., intended application or use of the shoe), etc. The insert can be composed of at least one or a combination of carbon fiber, plastic, natural fiber composites, metal, among other materials. In some cases, certain types of shoes may not require an insert or may include an embedded insert which the system can modify. For instance, the system can bolt or install a quick-release cleat directly into a hard sole, among other areas of a shoe. The type of application can include at least walking, running, jumping, among other activities intended for the shoe, for example. The system can utilize a template (e.g., shaped piece of material, such as metal, wood, plastic, etc.) to grind a cavity for the insert. The system can perform one or more preparation or adhering (e.g., gluing, soldering, taping, molding, nailing, etc.) steps to bond the parts of the shoe together. In this case, the parts can include the two split mid-sole and the insert of the shoe. Accordingly, the system can efficiently modify or assemble any pair of shoes to be compatible with the exoskeleton installation. The system can provide or install a quick-release cleat (e.g., QD, fixture, etc.) on the shoe to facilitate a disconnection (or connection) of the exoskeleton to the modified shoe.

The system can include multiple sizes of QDs (e.g., cleats) that can fit, install, connect, or bridge the exoskeleton onto the leg (e.g., or certain items hooked or worn on the leg) at a different angle. The QD can be located at the bottom of the exoskeleton, or configured at other portions of the exoskeleton. For instance, a large QD angle can clear or be distant from the ankle bone or footwear of the user, such as in configurations 4300B and 4300C). In another example, a smaller QD angle can bring the armature of the exoskeleton closer to the leg, ankle bone, or footwear of the user, such as in configurations 4300D and 4300E. A medium QD angle can be between the large QD angle and smaller QD angle, such as more distant from the leg than the smaller QD angle, while closer to the leg than the larger QD angle (e.g., configuration 4300A). Hence, the larger QD angle can move or displace the lower portion (e.g., the bottom) of the exoskeleton away from the leg, ankle bone, or footwear of the user. The QD or the QD angle can be configured, modified, or changed by the user, for instance, using one or more bolts or other adjustment mechanisms. The maximum or minimum QD angle can be predetermined based on a specification (e.g., standard) or configured by the manufacturer of the exoskeleton.

The system can include multiple sizes (e.g., length, width, height, or other dimensions) of shin levers to fit, bridge, or connect the exoskeleton to the leg of the user at a different angle. In some cases, the shin lever can be structured or configured to fit with a device, structure, material, or component worn or attached to the user. A larger shin lever can clear, move, or distance the top of the exoskeleton away from the leg or the calf muscle of the user by pushing the armature away from the leg. A shorter shin lever can pull, bring, or contract the armature of the exoskeleton closer to the leg, allowing the top of the exoskeleton to be closer to the leg. The shin lever can be adjusted by the user, such as using one or more screws, bolts, or other coupling mechanisms. For instance, the screws of the shin lever can be replaced by a thumbscrew, a lock pin, a twist-release, or any other tool-less mechanism. Hence, the system can allow easy-release or easy packing of the exoskeleton for transport.

In the example of FIG. 43, five configurations (e.g., configurations 4300A-E) of the QDs and shin levers are shown. Configuration 4300A can include a medium QD angle and extra-large shin lever. Configuration 4300B can include an extra-large QD angle and an extra-large shin lever. Configuration 4300C can include an extra-large QD angle and an extra-small shin lever. Configuration 4300D can include an extra-small QD angle and an extra-small shin lever. Configuration 4300E can include a small QD angle and a small shin lever. Other configurations or combinations of QD angles and shin levers can be utilized for fitting the exoskeleton to the user, such as based on at least the body size or type, or preference of the user.

The system can utilize or leverage the combination of the cleat (e.g., QD) and the shin lever to enable the exoskeleton to fit or couple to all body types, shapes, length, etc., or to use the exoskeleton with different equipment or apparatus. For instance, installation of the exoskeleton to a user wearing a ceratin boot and pants may require extra clearance compared to a user wearing a sneaker and shorts. Other clothing, accessories, or equipment of the user may contribute to the clearance required for fitting the exoskeleton. While a running exoskeleton (e.g., exoskeleton structured or configured for running) is used herein as an example, the system can include other types of exoskeletons to fit with the user using the QD and shin lever, such as an exoskeleton for walking, fast-walking, hiking, jogging, snowshoeing, cross-country skiing, backcountry skiing, snorkeling, etc.

FIGS. 44A-B illustrate a battery 4402 (e.g., sometimes referred to as a battery module, battery pack, battery component, power supply, group of battery cells, among other interchangeable terms). The battery 4402 can include similar features as or correspond to the battery module 3302. The battery 4402 (or at least a portion thereof) can be composed of one or more rigid materials. For example, the rigid battery can include a symmetrical design with one or more connectors. The battery 4402 can include a connector (e.g., a first connector) that allows for connection to the exoskeleton (e.g., on a first end of the battery 4402). The battery 4402 can include a connector (e.g., a second connector) that allows for charging of the battery 4402, such that the battery 4402 can power the exoskeleton and be charged concurrently. In some cases, the connector can include or refer to a port configured to connect with the exoskeleton and charge the battery 4402. The battery design (e.g., symmetrical design) can be used to fit the left or right exoskeleton (e.g., exoskeleton equipped on the left or right leg). Although described herein as having a symmetrical design, the rigid battery may include other designs for performing one or more similar operations, for example, charging and powering devices. The battery 4402 can include at least one of a foam tape or a non-extensible tape (e.g., kapton), among other materials used to assemble a battery brick from N individual cells without soldering. The N can represent the number of individual cells included or contained in the battery 4402.

The battery 4402 (or a portion thereof) can be composed of one or more flexible materials, such that the battery 4402 includes at least one flexible portion. For example, the battery 4402 can include a flexible printed circuit board (PCB). The flexible PCB can put or include multiple (or all) cells in series. The flexible PCB can expose individual cells' voltage on a balancing connector (e.g., shown in FIGS. 44A-B). The PCB geometry, copper, or other parts or components of the battery 4402 can be designed to support various bend cycles (e.g., a thousand, ten thousand, etc.) in at least one axis, and at least one sharp bend in the opposing axis (e.g., for each call and for the connector of the battery 4402). The flexible battery 4402 can include a symmetrical design. One or more connectors that are not used can be cut away, for instance, connectors that are not for the operation of the exoskeleton or charging of the battery 4402. The battery 4402 can include at least one mechanical structure with flexible members to allow different curve radii. In some cases, the battery 4402 can include a protection mechanism, such as to avoid or prevent over-bending of the PCB or in some cases under bending of the PCB (e.g., during certain movements by the user equipped with the exoskeleton). One or more of the battery 4402 can be worn at different areas of the body, such as the waist, calf, etc. The battery 4402 can be structured to provide flexibility matching different areas of the body. In some cases, full BMS can be built on the rigid PCB parts.

Each cell of the battery 4402 can include or be embedded with a connector to facilitate or allow replacement, such as due to failure of individual cells or end of life of the cells. For instance, an operator or user can disconnect a first cell (or first set of cells) and replace with a second cell (or second set of cells). The cells may be commercially available, such as COTS cells. In some cases, at least one cell may be a custom or modified cell. The battery 4402 can include multi-layer PCBs used to carry large peak currents (e.g., above 15 A, etc.) in a small package, with minimal thermal dissipation. The battery 4402 can include one or more cutouts in the PCB for weight reduction. In some cases, the battery 4402 can be a calf-mounted battery for integration with a shin pad or other equipment. In this case, a hook-and-loop (e.g., Velcro) belt can be used to attach to the shin pad, thereby holding the battery pack or battery module while equipping the shin pad. If a battery pack supports N cells, the battery 4402 can use 1 to N cells with one or more jumpers connected to unused cells. Hence, the system can allow lighter battery packs for specific applications. In some cases, the system can include firmware (e.g., customized or modified firmware for the specific battery) to support the charging of the battery 4402 while in electrical connection with the exoskeleton. The firmware may support batteries local to the exoskeleton (e.g., embedded batteries that cannot be disconnected).

In some cases, the rigid battery may include a rigid PCB with at least one predetermined or selected curvature on at least one portion of the PCB. In some implementations, at least a portion of the battery 4402 (or PCB) can be composed of both rigid and flexible materials, such that at least a first portion is rigid and a second portion is flexible (e.g., hybrid of stiff and flexible). In this case, the cells can be split into different groups, such as 2, 3, or 4 groups of cells. In some cases, the battery 4402 can include at least one fuse in series between two N cell packs. For instance, instead of using the same fuse on the positive and negative terminal, the battery 4402 may include or create two (e.g., N/2) cell packs. Hence, a 12 cell (e.g., 12S) battery 4402 can correspond to or be 2×6S batteries, such that the battery 4402 can be shipped or transported without restriction, for example.

The system may include pants customized or modified with one or more electrical conductors. The electrical conductors can allow waste or backpack-mounted batteries (e.g., among other types of mount) to power the exoskeleton or multiple exoskeletons. The electrical conductors can include or correspond to one or more cables. The cables can be removed from the pants (e.g., for washing the pants). The cables may be fished or retrieved in sewed channels. In some cases, the cables can be sewed in the pants and washed (e.g., the pants can be exposed to moisture, liquid, detergent, heat, among other external elements with the cables). In some cases, the cable can be sleeved when installed or embedded in the pants, such that the cable can be exposed to various elements (e.g., water, heat, etc.).

The exoskeleton as discussed herein (e.g., with the same design) can be compatible or used with multiple battery options. In some cases, at least one battery option can be used for various exoskeleton designs. For instance, the exoskeleton can be used with small, medium, or large local batteries. The exoskeleton can be used with small, medium, or large calf-mounted batteries. The exoskeleton can be used with small, medium, large, single, or double waist-mounted or backpack-worn batteries. In some cases, one battery 4402 can be used to power multiple exoskeletons (e.g., two, three, etc.).

An extender cable can be used to connect at least one battery 4402 to at least one exoskeletons. The extender cable can accommodate different body types. The extender cable may accommodate different pants or other equipment worn by the user.

The system identification and modeling can be used to improve controls for the exoskeleton. The system (or controller) can model the deformity of the shoe. For example, when torque (or other types of forces) is applied (e.g., during the user's movement), the insert or the sole of the shoe can deform. The controller can measure the torque applied to at least one portion of the shoe using an ankle angle sensor, or other sensors to measure the force. However, in some cases, the deformation may be read as a change of ankle angle, while the ankle of the user has not moved. Hence, the controller can implement or use a model to distinguish between a real angle and series compliance, such that the deformation can be associated with the movement of the user, for example. The controller can model the shoe insert and shoe sole compliance. The controller can be configured to generate, train, develop, or program the model (e.g., embedded controller of the exoskeleton). In some cases, the model can be generated or modified on a remote computing system, such as a remote server or a cloud computing system. The controller can use the model to compensate for the measured angle based on the applied torque. In some cases, the controller can use a look-up table to perform the compensation thereof.

The model can be developed offline. For instance, the controller can receive torque data applied to at least one of the shoe insert or shoe sole, measure the torque applied, repeat the process, and generate a model based on the measurements. The controller can simplify the model (e.g., filter certain data), provide or generate a code in a certain language (e.g., program in C based on the model), and use the model, code, or script in real-time. In some implementations, the controller can use system identification techniques or toolboxes, such as to compensate for the measured angle based on the applied torque.

The controller can calculate the model during runtime, such as on the embedded controller. In some cases, the controller can use a provided base model. In some other cases, a base model may not be provided. The controller can update the model in real-time (e.g., responsive to an event or certain actions by the user), such as based on walking data, running data, or other specific actions taken by the user (e.g., initiating a calibration procedure in an application and jump multiple times, or other calibration procedures). In some cases, the controller may update or reconstruct the model periodically or at predetermined time intervals for foam compression or for broken insert (e.g., wear or tear of the shoe insert). The controller can use the model to detect or identify a broken insert. The controller can use the model to inform that the sole foam has lost its properties (e.g., cushion, flexibility, etc.) and to alert the user to replace the shoe (e.g., or certain parts of the shoe).

Shoe choice can be used as controller input. For a certain control program, technique, or code, the perception of power or performance from the shoe may vary depending on the type of shoe, such as a difference in power between a thick, soft shoe compared to a thin, zero-drop shoe. The controller can receive an input of footwear selection to optimize or improve the user experience when selecting and using a shoe. The traction control program of the controller can compensate for the grip of the sole, which may be reduced subsequent to the usage of the footwear. Based on historical data (e.g., previous runs or other activities by the user), the controller can determine, recommend, suggest, or select optimal footwear for the user based on at least the characteristic of the foot contact to the shoe (e.g., heel strikers can be recommended with more padded shoes).

The system can use an optimal running gait to create an optimal running controller or update an existing controller. For instance, by measuring the movement of a good runner (e.g., or other activities), the system can inform the controller to replicate the movement motion on one or more other users, thereby teaching or guiding the users with proper running gait and form. Hence, the system can improve the controls for the controller of the exoskeleton by replicating at least some of the movements of at least one high-performing athlete.

The controller may be used to encourage a longer duration of the activity, such as running walking, fast-walking, etc. For instance, the controller can initiate assistance (e.g., nudge, push, etc.) for the user to move the user further forward. In some cases, the controller may decline or refuse to slow down (e.g., unless the user forces the controller to slow down by force stopping the motion) to encourage the user to maintain a steady pace, or in some cases, increase the pace. For instance, the controller may perform a transition by slowing down a run to a walking pace, or between a faster and slower pace. In some cases, the exoskeleton may shake when stopping, such as in response to an error or other irregularities. For instance, a machine (or other organisms) may buckle, shake, or vibrate in certain scenarios or configurations to signify that there is an irregularity (e.g., error, etc.). By emulating this behavior with the exoskeleton, the controller can create a sentiment that the stoppage is unintended by the exoskeleton.

In some cases, the controller can encourage a faster pace (e.g., running faster) for the user. The controller can initiate a powerful impulse that propels the user forward (or backward in certain cases). The controller can control the ankle angle to change the running gait, which can simulate the starting motion of individual steps, for example. The controller can allow the user to load the system by creating a virtual spring that simulates a bouncy characteristic for the device. The controller can be ready for an explosive launch, such as similar to a “launch mode” or “sport mode” of a vehicle.

The controller may encourage optimal running gait. For example, the controller can change the limb angle of the exoskeleton, such as by applying power in a way that the foot is always angled and never flat on the ground during the run. In another example, the controller may compensate power during specific phrases of the activity, such as preventing heel striking or preventing heel from contacting the ground. In further example, the controller can provide a smoothness score to the user via an application coupled or communicative with the controller. In some cases, the controller may send or provide suggestions to the user to increase the score and improve the running gait.

The user can adjust the controller, such as the settings and configurations of the controller. The program or code can match one or more technical parameters (e.g., dorsiflexion angle, maximum power plantar flexion, current controller proportional gain, etc.) to user perception (e.g., early, late, explosive, supportive, etc.). The user can increment or decrement the parameters. The user can enable or disable certain motions, transitions, or activities (e.g., running only, no walking, etc.). In some cases, the user can select the handling of the transitions (e.g., reactive or conservative, enabled or disabled, timings, the confidence of the transitions, etc.). For instance, the user can set the controller to reactive which may provide an enhanced or improved trail running experience. Other settings can be applied to the controller to improve the user experience based on the activities by the user.

The controller can be configured for injury rehabilitation. In certain cases, one leg of the user may be affected at a given time. The user may indicate to the controller that a specific leg (e.g., left or right) is injured and the type of injury (e.g., Achilles' tendonitis, shin splints, etc.) via the application connected to the controller. The controller can adapt the power profile of the exoskeleton to minimize the pain and loading (e.g., the load applied to the specific leg or body part), thereby assisting the rehabilitation process while allowing the user to stay active. The controller can even out or balance the load on both sides (e.g., both legs of the user) to prevent muscular imbalances, and to prevent the injury from creating other injuries (e.g., hip flexor of the other leg overcompensating, among others). In some cases, an upper body sensor may be used on the user. In this case, the controller may receive data from the upper body sensor informing the posture of the user's whole body. The controller and the application can provide a recovery plan to the user. The plan can be adjusted or updated based on at least measured data or user inputs (e.g., pain is relieving, increasing, decreasing, etc.).

The controller can include built-in training tools or plans for users. For instance, in a certain program, new athletes may alternate between walking and routing bouts over a time period (e.g., a few weeks, etc.) until the athlete completes a predetermined distance (e.g., 5 km, 10 km, etc.). To follow the plan, the user may print or receive instructions from the application (e.g., visual or audio cues). An exoskeleton can support the training plan and adjust to the plan accordingly. For instance, if the plan includes a walking bout, the controller can transition to walking mode, thereby responsively informing the user on the particular activity. In this case, the application may not be required. In some cases, the controller can ensure that when athletes are told to decrease the running pace, the user will slow down, such as by slowing down or stiffening the motion of the exoskeleton. In some cases, if the user's heart rate or form is deteriorating during a running bout, the controller can apply more power or compensate for the form by simulating one of at least the user's good form or other athletes' good form. The exoskeleton can provide form and technique feedback to the user, such as via the application or via guidance by the exoskeleton. The training plan can be customized based on the user's progress. In some cases, the user can input the activity length (e.g., running or walking, etc.). In response to receiving the activity distance or length, the controller can utilize the full capacity of the available battery energy on the particular activity, such as for the whole input distance.

In some cases, the controller may include advanced built-in training tools. For example, with running intervals of 4×400 meters, the exoskeleton can force the user to run at a given pace by imposing a cadence. Similarly, the user can use the exoskeleton to do cadence workouts. The exoskeleton controller can be used to inform cadence, gait, and form to the user (e.g., guiding the user through the activity), while minimizing positive power. For instance, the exoskeleton may not consume power or inform the user of the form while the user exhibits good form. In response to the user exhibiting bad form, the exoskeleton may consume power to guide or inform the user of a bad form. The controller may assist or guide the user on how to execute the desired activity (e.g., running form, walking form, etc.), without fully engaging in the activity for the user (e.g., running for the user). In some cases, the controller may facilitate the user's training by loading or increasing the difficulties for the user to run (e.g., making it harder to run). For instance, the controller or the exoskeleton can simulate hills or hard conditions (e.g., muddy trails, snowshoes, etc.) for the user to perform strength training. In some implementations, the exoskeleton may be used to perform the activities for the user, such as for users with injured limbs, or other conditions.

The system can include hardware, software, or a combination of hardware and software, among other electronics. The system can include a physical user interface (PUI) with various modes (e.g., three or more modes). The PUI can support an on, standby, or off mode, which can be a part of the exoskeleton (e.g., configured on the exoskeleton). In the on mode, the power of the exoskeleton can be on, as well as augmentation being on (e.g., enabled). In the standby mode, the power can be on with the augmentation set to off (e.g., disabled). In the off mode, the power and augmentation can be off. In some cases, the PUI can include a gradual mode. For instance, in the gradual mode, the power can be on with configurable augmentation from 0 to 100%, thereby providing granularity for the user. The system can include a boost mode. The boost mode can change the safety limits of the exoskeleton for a predetermined time period (e.g., 10 seconds, 20 seconds, etc.) to allow the user to run faster or provide the user with additional range of motion. The boost mode (e.g., or other modes) can be triggered by a button press, interaction with an application connected to the exoskeleton, or other interactive means. The system can include other modes to provide different abilities or functionalities to the exoskeleton.

The system can include one or more features for configuring the modes. For instance, the system can receive a signal or input to change the mode via a rotary switch, button press or click, button sequence, touch screen, slider switch, exoskeleton motion (e.g., certain motion performed by the user), among others. The system can include one or more LEDs or displays to provide visual feedback to the user (e.g., color, blinking, fading patterns, on and off lighting, text, notification, or other visual effects). The PUI (e.g., a single PUI or multiple PUI) can be used for one or more exoskeletons based on the configuration of the exoskeleton. The PUI can be connected to the exoskeleton via at least one of wires, Bluetooth connection (or other radio interfaces), infrared, among other communication interfaces. The PUI can include a local battery 4402 or be powered by the exoskeleton. For instance, the exoskeleton can charge the PUI's local battery 4402 or be a power source to the PUI. The exoskeleton can charge the PUI by wired connection, inductive charging, etc. In some cases, energy harvesting (e.g., motion, heat, light, etc.) can be used to charge the battery 4402 of the PUI (or the exoskeleton). The PUI may use power when the user is interacting with the interface, for instance, using a smart power management technique. In some cases, the PUI can be a smartwatch or other smart wearables equipped by the user.

The system can be connected to an application (e.g., mobile application, local application, or network application) that supports the same modes as the PUI. In this case, the system can be operated or configured to different modes via the application. The IMU can detect or measure that the motion of the exoskeleton satisfies (or does not satisfy) a threshold for a predetermined time period (e.g., less than 0.01 g of force for more than 5 minutes, etc.). In this case, if the IMU detects that the exoskeleton is below the threshold for more than a predetermined time, the exoskeleton can turn off or power down to preserve batteries.

The system can include IMU-based settings. For example, the user can undergo a certain movement (e.g., swinging movement, swiping movement, stomping their foot at a set frequency for a certain number of steps, etc.) to enter settings mode. The exoskeleton can use the IMU and one or more other available sensors (e.g., ankle angle sensor, etc.) to recognize or detect the predetermined movements configured to enter the setting mode or set the exoskeleton into the setting mode. The left and right exoskeletons may communicate with each other to synchronize the settings. The settings may be adjusted on one exoskeleton and then communicate to the contralateral side, for example. In some cases, the settings can be adjusted for individual exoskeletons. Each side or exoskeleton may be responsible for different settings. For example, the right exoskeleton may control power level and the left exoskeleton may control the power type for individual exoskeletons or both exoskeletons.

The exoskeleton can sense or detect any movement to enter or exit the settings mode or adjust one or more parameters or configurations within the settings mode. The types of movements that can be discerned by the exoskeleton can include at least stomping, sliding feet, tapping on the exoskeleton (e.g., with finger or hand), or twisting of the foot (e.g., angle of twist can be used as a power knob and the power setting can be proportional to the angle of the twist). the sliding movement can include forward sliding, horizontal sliding, or a combination of forward, medial, lateral, etc. to enable the user to navigate in a virtual 2D space. In some cases, the motions detected by the exoskeletons can allow the user to navigate in a 3D space, such as detecting the x-axis, y-axis, and z-axis movements. Further, the types of movement can include at least ankle angle or shank angle, when user clicks their heels together, among others. The ankle angle can include a squat motion which may indicate a power level, or the user can swing their leg forward (or backward) and the angle of the shank may determine the setting level. The clicking of the heels can indicate a bilateral communication between the left and right exoskeletons, such as to enable or initiate synchronization. The left and right exoskeletons may sense the wisting along an axis perpendicular to the ground, as well as sensing a synchronized pulse received by both the exoskeletons. The synchronization may consider or account for certain latencies known to occur between the left and right communications.

In some cases, the system can include an exoskeleton boot (e.g., exoBoot) which may indicate that the settings mode has been entered via a feedback indication. The feedback can include at least one of visual (e.g., LED blinking pattern, color change, etc.), audio (e.g., built-in speaker or pulse width modulation (PWM) frequency on motor can be changed to generate or create sound feedback), or tactile feedback (e.g., a motor can be configured or programmed to vibrate, where the vibration is modulated by amplitude, frequency, or duration).

Responsive to accessing the settings mode, the user may use additional movement to scroll through the settings. For instance, one stomp can indicate or be low power, two stomps can be normal power, and three stomps can be high power configuration for the exoskeleton. In some cases, twisting of the foot can indicate the power level of the exoskeleton. The exoskeleton can use a feedback mechanism to indicate to the user that the settings have been successfully changed (e.g., updated, modified, implemented, etc.) or abandoned (e.g., successfully canceled, timeout, etc.). The feedback may also indicate that one or more settings have been selected. For example, if the user is setting the power level, the exoskeleton may vibrate in discrete bursts to indicate which power level was selected. In this case, 1 bust may indicate low power selection, 2 bursts may indicate normal power, and 3 bursts may indicate high power selection. The exos may also use certain techniques (e.g., accidental press technique or monitoring system or program) to detect when the settings mode has been accidentally triggered. For example, if the settings mode is triggered and walking steps are identified in response to triggering the settings mode, all setting changes may be ignored. Otherwise, the user can provide a predetermined motion (e.g., triple stomps or shakes) to indicate that the changes should be implemented or not canceled.

The system can include smart under-voltage lockout (UVLO). The UVLO technique, program, or system can compare the instantaneous battery voltage to a constant and turn the system off responsive to the battery 4402 lower than a threshold (e.g., a minimum capacity threshold). Further, the UVLO may use a filtered value to reject transients. For instance, a smart UVLO system can utilize the controller state (e.g., activity, mode, gait phase, commanded, or measured torque, etc.), current and voltage measurements, or a system model (e.g., battery state, series resistance and impedance, efficiency, etc.) to reject transients and use the actual state of charge to determine a low voltage. The amount of battery energy used during a session can be used to inform or indicate the battery state. For example, if the battery current and voltage sensor measure an average battery power of 5 W over 2 hour, the smart UVLO can determine that the battery level has been reduced by at least 10 W per hour. The smart UVLO can run on the BMS, the embedded controller, or partially on both the BMS and the embedded controller. The smart UVLO can be redundant or complementary to the BMS or the embedded controller.

The system can include or perform hardware power management. The system can use the controller state and sensor inputs to adjust the clock frequency of the microcontroller and control loop frequency. The system may use similar inputs to change the hardware state. The system can include one or more electronic switches (e.g., point of load switches) for disabling sub-circuit during standby or during specific gait phases. Similarly for sensors, for instance the system can power off the ankle angle sensor when the system is on standby (e.g., responsive to entering or during standby mode). In response to the IMU detecting motion, the controller can power the ankle angle sensor on. Microcontroller cores can be enabled/disabled.

The system can perform dual-range current sensing with a single sensor. For instance, the one sensor (e.g., via a resistor, hall effect, etc.) can measure a current across one or more electrical components. Two amplifiers can provide 1:1 and 1:2 range, for example, both may be converted simultaneously by two analog-to-digital converters (ADCs). The system can use a code or technique to determine the ADC output to use at a given time based on at least one of the previous measurement (e.g., measurement from the one sensor), trend, hysteresis, controller state, among others.

Referring to FIG. 45, depicted is a block diagram of a system 4500 for augmented motion. The components of the system 4500 discussed herein can include or be composed of hardware, software, or a combination of hardware and software components. The system 4500 can include, interface with, facilitate the operation of, or communicate with one or more networks 4502, at least one exoskeleton 100, at least one client device 4504, or at least one data processing system 4506. For example, the system 4500 can include, interface with, communicate with, or facilitate the operation or performance of one or more system or component depicted in FIGS. 1-44. The one or more components (e.g., exoskeleton 100, client device 4504, or data processing system 4506) of the system 4500 can establish communication channels or transfer data via the network 4502. For example, the data processing system 4506 can communicate with the client device 4504 through a first network and the data processing system 4506 can communicate with the exoskeleton 100 via a second network, etc. In some cases, the first network and the second network can be the same network 4502. In some other cases, the first network and the second network may be different networks bridging or enabling communication between different devices or components of the system 4500.

The network 4502 can include computer networks such as the Internet, local, wide, metro or other area networks, intranets, satellite networks, other computer networks such as voice or data mobile phone communication networks, and combinations thereof. The network 304 may be any form of computer network that can relay information between the one or more components of the system 4500, such as between the exoskeleton 100, the client device 4504, or the data processing system 4506. In some implementations, the network 4502 may include the Internet and/or other types of data networks, such as a local area network (LAN), a wide area network (WAN), a cellular network, a satellite network, or other types of data networks. The network 4502 may also include any number of computing devices (e.g., computers, servers, routers, network switches, etc.) that are configured to receive and/or transmit data within the network 4502. The network 4502 may further include any number of hardwired and/or wireless connections. Any or all of the computing devices described herein (e.g., exoskeleton 100, client device 4504, or data processing system 4506, etc.) may communicate wirelessly (e.g., via WiFi, cellular, radio, etc.) with a transceiver that is hardwired (e.g., via a fiber optic cable, a CAT5 cable, etc.) to other computing devices in the network 4502. Any or all of the (e.g., computing) devices described herein (e.g., exoskeleton 100, client device 4504, or data processing system 4506, etc.) may also communicate wirelessly with the computing devices of the network 4502 via a proxy device (e.g., a router, network switch, or gateway).

The system 4500 can include or interface with at least one client device 4504 (or various client devices 4504). Client device 4504 can include at least one processor and a memory, e.g., a processing circuit. The client device 4504 can include various hardware or software components, or a combination of both hardware and software components. The client device 4504 can be constructed with hardware or software components. For example, the client device 4504 can include, but is not limited to, a television device, a mobile device, smart phone, personal computer, a laptop, a gaming device, a kiosk, or any other type of computing device.

The client device 4504 can include at least one interface for establishing a connection to the network 4502. The client device 4504 can communicate with other components of the system 4500 via the network 4502, such as the exoskeleton 100 or the data processing system 4506. For example, the interface of the client device 4504 can include hardware, software, features, and functionalities of at least a communication interface(s) or user interface. In some cases, the client device 4504 can communicate with other client devices.

The client device 4504 can include, store, execute, or maintain various application programming interfaces (“APIs”) in the memory (e.g., local to the client device 4504). The APIs can include or be any types of API, such as Web APIs (e.g., open APIs, Partner APIs, Internal APIs, or composite APIs), web server APIs (e.g., Simple Object Access Protocol (“SOAP”), XML-RPC (“Remote Procedure Call”), JSON-RPC, Representational State Transfer (“REST”)), among other types of APIs or protocol.

The client device 4504 can include a display device configured to provide a graphical user interface (GUI) or visual presentation to the user (or subject). The client device 4504 can request information from the subject, such as shoe type, shoe size (or dimension), shoe identifier, at least one type of activity about to be performed by the user, among other types of information discussed herein. The client device 4504 can display one or more predetermined or configured sets of commands to the subject for controlling at least one component (e.g., motor 1601, 1703) of the exoskeleton 100. For example, the client device 4504 can provide the subject with at least one set of commands for configuring the energy output (e.g., power optimization options), the type of activity (e.g., optimization for certain types of activity), or other performance profile configuration for the exoskeleton 100. The subject can interact or interface with the exoskeleton 100 through an application of the client device 4504. In some cases, the client device 4504 can be a part of the exoskeleton 100, such as an embedded device of the exoskeleton 100. In various aspects, the client device 4504 can receive data (e.g., sensor data or measurement data) from the exoskeleton 100 (or one or more components of the exoskeleton 100). The client device 4504 can transmit or relay data to the data processing system 4506 for processing. In some cases, the client device 4504 can receive instruction or command from the data processing system 4506 to execute for controlling or configuring the exoskeleton 100.

The system 4500 can include at least one data processing system 4506. The data processing system 4506 can sometimes be referred to as or correspond to a controller (e.g., for controlling the exoskeleton 100). The data processing system 4506 can be composed of hardware or software components, or a combination of hardware and software components. The data processing system 4506 can be remote from the exoskeleton 100 and the client device 4504. In some cases, the data processing system 4506 can be a part of at least one of the exoskeleton 100 or the client device 4504, such as an embedded system of the exoskeleton 100. The data processing system 4506 can receive data or information from the exoskeleton 100 or the client device 4504. The data processing system 4506 can communicate with other devices within the network 4502. The data processing system 4506 can transmit information (e.g., processed information) to the client device 4504. The data processing system 4506 can transmit a command or instruction to client device 4504 for controlling the exoskeleton 100 or directly to the exoskeleton 100.

The data processing system 4506 can include various components for processing information. For example, the data processing system 4506 can include at least one interface 4508, at least one data collector 4510, at least one footplate selector 4512, at least one performance configurator 4514, at least one model manager 4516, at least one motor controller 4518, and at least one database 4520. Individual components (e.g., interface 4508, data collector 4510, footplate selector 4512, performance configurator 4514, model manager 4516, or motor controller 4518) of the data processing system 4506 can include or be composed of hardware, software, or a combination of both hardware and software components. Individual components can be in electrical communication with each other. For instance, the interface 4508 can exchange data or communicate with the data collector 4510, footplate selector 4512, performance configurator 4514, model manager 4516, or motor controller 4518. The one or more components (e.g., interface 4508, data collector 4510, footplate selector 4512, performance configurator 4514, model manager 4516, or motor controller 4518, etc.) of the data processing system 4506 can be used to perform features or functionalities discussed herein, such as selecting footplate, configuring the performance of the exoskeleton 100, selecting a model, controlling the motor (e.g., motor 1703) of the exoskeleton 100, among others. The data processing system 4506 can include other components (e.g., processors and memory) to perform features and functionalities described herein. In some cases, the one or more components of the data processing system 4506 can correspond to the controller for controlling or processing data to control the exoskeleton 100. The data processing system 4506 can be electrically connected to one or more components of the exoskeleton 100, such as to the motor 1703 or the battery 4402.

The interface 4508 can refer to a network interface card (NIC). The interface 4508 can be one of at least a physical interface 4508 or a virtual interface 4508. The type of the interface 4508 may be indicated by a configuration file during deployment of the data processing system 4506 or modified during execution of the data processing system 4506. The interface 4508 can interface with the network 4502, devices within the system 4500, among others. The interface 4508 can include features and functionalities to interface with the aforementioned components. For example, the interface 4508 can include standard telephone lines LAN or WAN links (e.g., 802.11, T1, T3, Gigabit Ethernet, Infiniband), broadband connections (e.g., ISDN, Frame Relay, ATM, Gigabit Ethernet, Ethernet-over-SONET, ADSL, VDSL, BPON, GPON, fiber optical including FiOS), wireless connections, or some combination of any or all of the above. Connections can be established using a variety of communication protocols (e.g., TCP/IP, Ethernet, ARCNET, SONET, SDH, Fiber Distributed Data Interface (FDDI), IEEE 802.11a/b/g/n/ac CDMA, GSM, WiMax and direct asynchronous connections). The interface 4508 can include at least a built-in network adapter, network interface card, PCMCIA network card, EXPRESSCARD network card, card bus network adapter, wireless network adapter, USB network adapter, modem, or any other device suitable for interfacing one or more devices within the system 4500 (or network 4502) to any type of network capable of communication.

The data collector 4510 can collect, receive, or obtain data or information from one or more components within the network 4502, such as the exoskeleton 100 or the client device 4504. The data collector 4510 can receive an indication of a type of footwear (e.g., footwear 600 or shoe) or one or more characteristics of the footwear in which the footplate is inserted or to be inserted. For example, the data collector 4510 can receive an identifier for the footwear from a user interface of the client device 4504. Based on the identifier, the data collector 4510 can identify the type of footwear by performing a lookup in the database 4520 (e.g., local database or remote database from the data processing system 4506) with the identifier. The data collector 4510 can identify information associated with the footwear in the database 4520, such as the dimension, type, material(s), one or more characteristics, or other information associated with the footwear. In some cases, the data collector 4510 can receive information associated with the footwear from the client device 4504 (e.g., user input data via the user interface of the client device 4504).

The type of the footwear can include at least one of a running footwear, a hiking footwear, a cross-training footwear, a basketball footwear, a boot, or a dress footwear, among others. The dimension of the footwear can include at least one of the manufactured size of the footwear, the length, width, or height of the sole of the footwear, etc. The material of the footwear can indicate rigidity, flexibility, or sturdiness of at least a portion of the footwear. The one or more characteristics (e.g., received via the user interface of the client device 4504) can include at least one of a thickness of the sole, a grip of an outsole of the footwear, a softness of the sole, or a rigidity of the sole, among others.

The data collector 4510 can identify, determine, or receive an indication of an activity (e.g., type of activity) to be performed with the footwear. The activity can include at least one of running, walking, or hiking, among others. The data collector 4510 may identify the type of activity based on the characteristic or the type of footwear (e.g., dress footwear for walking, sports footwear for running or walking, or boot for walking or hiking). In some cases, the data collector 4510 can receive an indication of the activity for the footwear from the client device 4504 via the user interface. For example, the subject can be provided with options for the types of activity to be performed using the footwear (or using the exoskeleton). In some cases, the data collector 4510 can identify both the type of footwear and the activity to be performed with the footwear.

In some cases, the data collector 4510 can receive an indication of a mode for augmentation. The mode for augmentation can be a part of a performance profile. For example, the indication of the mode for augmentation can include a performance mode configured to reduce the rate at which the electric motor transitions from augmented running to augmented walking (e.g., decrease the rate at which the subject slows down from running to walking), such as to encourage running at a steady pace. The mode for augmentation may include at least one of running, hiking, walking, launch mode, encouragement mode, or cast mode. The running mode may assist the subject in maintaining the running pace, increase the speed of the subject, or notify (e.g., vibration) the subject to increase the running pace. The hiking mode or walking mode can assist with the movement or motion of the subject.

Further, the launch mode (e.g., maximum power mode) can facilitate the output of the maximum power for facilitating the movement of the subject. In the launch mode, the data processing system 4506 (e.g., performance configurator or model manager 4516) can learn the maximum speed (or momentum) of the subject, thereby enabling the exoskeleton 100 to facilitate the movement to maintain the maximum speed or enhance the speed of the subject beyond the maximum speed without the exoskeleton. The data processing system 4506 can learn the maximum speed (or acceleration) the subject can handle, such as to avoid injury or falling. For instance, the data collector 4510 can collect measurement data from the exoskeleton 100 or the client device 4504 to determine the acceleration or speed suitable for the subject.

The encouragement mode can be configured to encourage or assist the subject with the activity. For instance, to assist with the running, the data processing system 4506 (e.g., the motor controller 4518) can maintain or reduce the rate at which the motor 1703 transition from augmented running to augmented walking. In some cases, the data processing system 4506 can command the motor 1703 (or at least one other component of the exoskeleton 100) to vibrate, indicating a reduction in speed or encouraging the subject to maintain a walking or running pace (e.g., configurable by the subject via the user interface of the client device 4504). The cast mode can assist the user with walking (or any other movement), for instance, for rehabilitation (e.g., subject with a cast), decreasing stress on the body part (e.g., the leg) during movement, or (e.g., entirely) augmenting the movement or motion of the subject.

In various aspects, the data collector 4510 can detect, via a sensor of the exoskeleton 100, a level of activity of the subject. The level of activity can be stored in a data log, such as indicating the performance of the subject. The data collector 4510 can detect a rate of motion of the subject, such as the rate or pace at which the subject walk or run. The data collector 4510 can detect, via a sensor that monitors motions of the ankle joint (or other body parts of the subject), a gesture performed by the subject. The gesture can be any type of motion, such as tap(s), swing(s), stomp(s), etc., predetermined or predefined by the subject or the manufacturer (or administrator) of the exoskeleton 100 or the data processing system 4506. The data collector 4510 can perform a lookup in the database 4520 with the detected gesture to identify or obtain a respective command corresponding to the gesture. In this case, the gesture can e associated with a type of command for execution, for instance, by the motor controller 4518.

The footplate selector 4512 can determine or select the footplate (e.g., composite plate 205) for insertion into a sole of the footwear to modify the footwear for augmentation. For example, the footplate selector 4512 can receive a request to modify the footwear for augmentation. The footplate selector 4512 can select the footplate based on the size of the footwear, such that the footplate have a size of less than or equal to the size of the footwear. The footwear can include a sole formed of an insole and an outsole. The footplate can be inserted in between the insole and outsole. The footplate can include multiple portions, which may be composed of similar or different materials. For simplicity, the footplate can include two portions (e.g., a first and second portions), such as provided in the examples herein. In various aspects, the footplate can include more than two portions composed of different materials or constructions.

The first portion of the footplate can extend along at least a first portion of the sole. The second portion (e.g., composite material 505) of the footplate can extend along at least a second portion of the sole. The first portion of the footplate can include greater rigidity than the second portion of the footplate. The first portion can be proximate to the heel of the footwear. A mounting component (e.g., mounting tab 620) can be connected to the first portion of the footplate. The mounting component can be configured to couple to a bracket to receive force from an actuator and apply the force to the first portion of the footplate via an axis of rotation about an ankle to augment motion, for example.

Subsequent to determining at least one of the size or materials for the footplate, the footplate selector 4512 can select the footplate. The footplate selector 4512 can provide an indication of the selected footplate for insertion between the insole and the outsole of the footwear. For instance, the first portion of the footplate located proximate to the heel of the footwear can have greater rigidity compared to the second portion. The mounting component can protrude at least partially from the sole of the footwear for coupling with other portions of the exoskeleton 100.

In some cases, prior to insertion or selection of the footplate, the footplate selector 4512 can identify a type of footwear to use for augmentation. The type of footwear can indicate at least the size or activity to be performed with the footwear. The footplate selector 4512 can determine the size (e.g., length) of the footplate based on the type of footwear. In this case, the footplate selector 4512 can select the footplate with the determined length for insertion into the sole of the footwear. In some cases, the footplate selector 4512 can determine, based on the type of the footwear, the size of the footplate such that the footplate terminates behind a metatarsal joint of the foot on which the footwear is worn. The footplate may terminate at other portions of the foot of the subject. The footplate selector 4512 can determine the activity to be performed with the footwear, such as based on the type of footwear or the one or more characteristics associated with the footwear. The footplate selector 4512 can determine, prior to insertion, the length for the footplate based on the activity to be performed. The footplate selector 4512 can select the footplate with the length for insertion into the sole of the footwear, such that the footplate is suitable for the activity (e.g., footplate with greater rigidity for hiking, lower rigidity for walking, among other balances).

In various configurations, the footplate selector 4512 can identify a type of the footwear and an activity to be performed with the footwear. Based on the type of footwear an the activity, the footplate selector 4512 can determine, prior to insertion, a length for the first portion of the footplate and a length for the second portion of the footplate. For instance, the length of the first portion can be longer than the second portion or vice versa. The footplate selector 4512 can determine an amount of rigidity for the second portion of the footplate based on the type of the footwear and the activity (e.g., less rigidity for walking activity and higher rigidity for hiking activity). The footplate selector 4512 can select, for insertion into the sole, the footplate having the determined length for the first portion, the determined length for the second portion, and the determined amount of rigidity for the second portion, for example.

The footplate selector 4512 can select the second portion of the footplate to be thinner than the first portion of the footplate. In some cases, the footplate selector 4512 may select the first portion that is thinner than the second portion. The second portion of the footplate can at least partially overlap (or entirely overlap) with the first portion of the footplate. The footplate selector 4512 can select, based on a type of the footwear or an activity to be performed with the footwear, the mounting component configured to protrude from a rear of the footwear. For example, with the type of footwear or activity indicating a higher amount of stress or force applied to the mounting component or the footplate, sturdier materials can be used for the mounting component.

In some cases, based on a type of the footwear or an activity to be performed with the footwear, the footplate selector 4512 can select the mounting component configured to protrude from a side at a heel of the footwear that is different from a rear of the footwear. The footplate selector 4512 can select the actuator configured to couple to a lower limb of a subject wearing the footwear. The actuator can couple below a knee of the lower limb of the subject.

The performance configurator 4514 can be configured to determine a performance profile for operating the electric motor (e.g., motor 1703) of the exoskeleton 100. The performance configurator 4514 can determine or select the performance profile based on the number of battery cells or a type of battery cell of the battery pack (e.g., a group of various battery cells). The battery pack can be integrated in the shin pad. In some cases, the battery pack can be coupled to a calf behind the shin (e.g., behind the shin pad) of the subject. The battery pack can be a flexible battery.

The performance configurator 4514 can establish or set a duty cycle for a switched-mode power supply (e.g., part of the battery pack) in accordance with the determined performance profile. By establishing the duty cycle, the performance configurator 4514 can cause the switched-mode power supply to convey power from the battery pack to the electric motor to generate the torque about the axis of rotation of an ankle joint of the subject. For example, the motor 1703 can generate and apply force to the footplate inserted into the sole of the footwear. The motor 1703 can generate the force responsive to obtaining the power conveyed from the battery pack in accordance with the duty cycle.

In some cases, the performance configurator 4514 can receive an indication to enter an emergency mode (e.g., from at least one component of the exoskeleton 100 or from the client device 4504 monitoring the status of the exoskeleton 100). For example, the emergency mode can be triggered in response to the capacity of the battery being less than or equal to a threshold (e.g., 10%, 5%, or 3% based on a configuration by the subject or the administrator). In another example, the emergency mode can be triggered by the subject via the user interface on the client device 4504. In yet another example, the emergency mode can be triggered based on the health status of the subject, such as a heart rate (e.g., at or above a threshold beats per minute (BPM)) obtained from a heart rate monitor on the client device 4504 or at least one sensor of the exoskeleton 100. The performance configurator 4514 can allow or enable, responsive to the indication to enter the emergency mode, the switched-mode power supply to discharge the battery pack beyond a minimum limit established for the battery pack. For example, the performance configurator 4514 can cause the motor 1703 to enter an idle mode or provide enough force to the footplate to move the footplate similar to the movement of the subject.

In another example, an emergency mode may refer to a mode for maximizing the energy output by the motor 1703. In this case, the minimum limit may correspond to an initial upper limit for energy (or power) output. Hence, the performance configurator 4514 can unlock the initial upper limit or set a second minimum limit (or threshold) for the maximum energy output, where the second minimum limit is greater than the initial minimum limit. In this case, the performance configurator 4514 can provide additional support to augment motion for the subject by outputting more power to the motor 1703.

In some cases, the performance configurator 4514 can detect that the battery pack has been replaced with a second battery pack including a second group (e.g., second battery pack) of battery cells. The performance configurator 4514 may determine, based on the second number of battery cells (e.g., second amount of battery cells) or the second type of battery cells associated with the second battery pack, a second performance profile for the electric motor. According to the second performance profile, the performance configurator 4514 can establish a second duty cycle for the switched-mode power supply to cause the switched-mode power supply to convey power from the second battery pack to the electric motor to generate the torque about the axis in accordance with the determined second performance profile. The second performance profile can be different from the (e.g., first or initial) performance profile. For example, the second battery pack may include more cells (e.g., higher capacity) or a higher efficiency type of battery cells compared to the first battery pack. In this case, the second performance profile can set a higher duration of on-time for the second battery pack relative to the first battery pack, or vice versa.

The type of battery cell can indicate at least one of a capacity or a maximum peak current. Hence, the performance profile can be configured to optimize the lifetime relative to energy output, or optimize energy relative to lifetime. For example, a lower duty cycle (e.g., relatively less on-time or less frequent provision of energy) can be used for optimizing the lifetime (e.g., of the battery) relative to the energy output. In another example, a higher duty cycle (e.g., relatively less downtime, more provision of energy, higher amplitude, or more frequent on-time) can be used for optimizing energy relative to the lifetime.

In some cases, a second battery pack including the second group or set of battery cells can be electrically connected with the electric motor (e.g., motor 1703) and the data processing system 4506 (e.g., controller). The second battery pack can include a greater number of battery cells relative to the (e.g., first) battery pack. In this case, the performance configurator 4514 can cause the switched-mode power supply to discharge the battery pack with the duty cycle greater than a threshold in accordance with the performance profile, such as the performance profile configured for a greater number of battery cells. In some cases, the performance configurator 4514 can select a second performance profile for the second battery pack. The performance configurator 4514 can cause the switched-mode power supply to discharge the battery pack with a second duty cycle less than the threshold in accordance with the second performance profile. In this case, the second duty cycle can be used to conserve or prolong the remaining battery capacity. In some cases the second duty cycle can include less downtime, thereby increasing the energy output to the motor 1703, for example.

The performance configurator 4514 can receive an indication of an activity to be performed by the subject. Based on the indication of activity, the performance configurator 4514 can select at least one of the first battery pack associated with the performance profile, or the second battery pack associated with the second performance profile, to deliver power to the electric motor to augment the activity. For example, with a less intensity activity (e.g., activities that may require an amount of force at or below a threshold), the performance configurator 4514 can select one of the battery packs associated with a lower performance profile (e.g., for walking). In another example, with a high-intensity activity (e.g., activities that may require an amount of force at or above the threshold), the performance configurator 4514 can select one of the battery packs associated with a higher performance profile (e.g., for running, hiking, sprinting, among others).

In some cases, the second battery pack can be electrically connected to a second motor to provide torque to apply force via a second lever to a second footplate to generate torque about an axis of rotation of a second ankle joint of the subject. The second motor, second lever, or second footplate can include or be composed of similar materials as the first motor, first lever, or first footplate, for example. In this case, the first motor can be associated with a first body part (e.g., first leg) and the second motor can be associated with a second body part (e.g., second leg) of the subject. The performance configurator 4514 can be configured to balance, responsive to the battery pack electrically connected with the second battery pack, a state of charge of the battery pack with a state of charge of the second battery pack. For example, the battery packs can be charged at a similar rate. In another example, the battery packs can output energy to the respective motors at a similar duty cycle, amplitude, interval, etc. In this case, the first and second battery packs can synchronize with each other. In some other cases, the battery packs may not operate synchronously, such that one of the battery packs may output more (or less) power than the other. In some cases, the performance configurator 4514 can decrease (or increase) the power output or the duty cycle of at least one of the battery packs to maintain a similar battery capacity between the battery packs.

The model manager 4516 can obtain information from the data collector 4510, such as the one or more characteristics of the footwear in which the footplate is inserted or the indication of the mode for augmentation. Based on at least one of the one or more characteristics or the mode for augmentation, the model manager 4516 can select at least one model for generating torque (e.g., torque generation). The model can be predetermined or configured to include instructions for controlling the energy (or power) output from the motor 1703. The mode for augmentation can include at least one of running, hiking, walking, launch mode, encouragement mode, or cast mode, among others. The model manager 4516 (or the motor controller 4518) can use the model to instruct or command the electric motor to generate torque. The model manager 4516 can select the model configured to provide a certain level of energy output suitable for the intended activity performed by the subject.

For example, the model can be associated with the mode for augmentation (e.g., part of the performance profile) or the one or more characteristics of the footwear (e.g., indicative of the type of activity to be performed). Different models can provide or indicate different configurations of the power delivery instructions. In some cases, the model can indicate or include the duty cycle, maximum (or minimum) energy output threshold, etc. With augmentation mode or the characteristic indicative of a higher torque generation (e.g., running or sprinting), the model manager 4516 can select the model configured for the motor controller 4518 to instruct the motor 1703 to provide a higher energy output (e.g., relative to an indication of low torque activity). In another example, with augmentation mode or the characteristics indicative of a lower torque generation or optimizing energy efficiency or capacity (e.g., walking or hiking), the model manager 4516 can select the model configured for the motor controller 4518 to instruct the motor 1703 to provide a relatively moderate or low energy output (e.g., relative to the indication of a high torque activity). The high torque activity or the low torque activity can refer to an activity that may experience or require a relatively higher force generation from the motor to drive the footplate to perform the desired activity or a relatively low (or moderate) forge generation for the footplate, respectively.

In some cases, the model manager 4516 can select the model based on the level of traction or rigidity. For example, the model manager 4516 can obtain data from the data collector 4510, such as the first level of traction of an outsole of the footwear and a first level of rigidity of the sole identified via a lookup of the footwear identifier. Further, the data collector 4510 can provide the measured data from the exoskeleton 100 (e.g., via an ankle angle sensor) or measurement of torque applied by the electric motor to the model manager 4516. The measurement data can include at least one of a second level of traction of the outsole or a second level of rigidity of the sole. The second level of traction may be less than the first level of traction or vice versa. The second level of rigidity can be less than the first level of rigidity or vice versa. In this case, the model manager 4516 can select a second model based on the second level of traction and the second level of rigidity with which to instruct the electric motor to generate torque. The second model can be different from the (e.g., first) model, where the first model may be based on the first level of traction and rigidity associated with the footwear (e.g., new footwear).

For example, with less level of traction or rigidity, the model can be configured to generate less torque to prevent losing traction or grip during the activity. In some cases, if at least one of a level of traction or a level of rigidity is at or below a predetermined threshold configured by the administrator (e.g., indicative of a potential accident), the model can be configured to notify the subject (e.g., vibration or notification via the user interface), encourage the subject to slow down or maintain the current speed (e.g., reducing power output), or decrease the rate of acceleration (e.g., decrease the rate of change from augmented walking to augmented running).

The motor controller 4518 can transmit or provide one or more instructions, commands, or indications to at least one of the client device 4504 or the electric motor of the exoskeleton 100 to generate torque. For example, the motor controller 4518 can control the exoskeleton 100 via communication with the client device 4504. In another example, the motor controller 4518 (or the data processing system 4506) can be in electrical communication with the motor 1703 or the battery module 3302 of the exoskeleton 100, such that the motor controller 4518 can directly control or provide instructions to the electric motor. The instructions to generate torque can be based on or according to the model selected by the model manager 4516.

In some cases, the motor controller 4518 can set the clock rate of the one or more processors of the data processing system 4506, such as based on the model for augmentation. For example, for walking or hiking mode, among others, that may not involve frequent changes or fast motion by the subject, the motor controller 4518 may set a lower clock rate to conserve the battery capacity (e.g., reduce energy provided to the data processing system 4506 (or controller) local to the exoskeleton 100, the sensor(s), or one or more processors associated with the exoskeleton 100). For example, the data collector 4510 can receive data indicative of a level of activity of the subject via at least one sensor (e.g., motion sensor). If the level of activity (e.g., speed) of the subject is less than (or equal to) a threshold, the motor controller 4518 can decrease the clock rate of the one or more processors (e.g., of the controller or the data processing system 4506). In another example, if the detected level of activity of the subject is greater than (or equal to) the threshold, the motor controller 4518 can increase the clock rate of the one or more processors to increase the resolution of the logged, recorded, or collected data.

In various cases, the motor controller 4518 can set a sample rate of one or more sensors of the exoskeleton 100 based on a level of activity of the subject. For instance, with a higher level of activity (e.g., intensity or speed of the subject), the motor controller 4518 can increase the sample rate of the sensor to capture more data. In other instances, with a lower level of activity (e.g., less intensive activity), the motor controller 4518 can decrease the sample rate of the sensor to decrease the power consumption and extend the battery capacity.

In some cases, the motor controller 4518 can receive an indication of the rate of motion of the subject from the data collector 4510. The motor controller 4518 can determine that the rate of motion is less than (or equal to) a threshold. For example, the subject may set the threshold for the minimum walking or running speed. The motor controller 4518 may instruct (e.g., in encouragement mode) the electric motor (e.g., one or more motors) to vibrate responsive to the detection of the rate of motion less than the threshold. Hence, the motor controller 4518 can notify or encourage the subject to maintain or increase the speed.

The motor controller 4518 can receive an indication of a gesture from the data collector 4510. The data collector 4510 detects the gesture performed by the subject via a sensor of the exoskeleton 100 that monitors motions of the ankle joint. The gesture can be stored in the database 4520. The motor controller 4518 (or the data collector 4510) can identify a command corresponding to the detected gesture. The gesture can include at least one of a wave, tap, stomp, slide, or pointing, among others. The motor controller 4518 can execute the command to instruct the electric motor in accordance with the command. For instance, the command can include at least one of switching the type of activity, performance profile, power optimization configuration, or powering the exoskeleton on or off, etc.

The database 4520 can include include, store, or maintain various data discussed herein to control or operate the exoskeleton 100. The database 4520 can be implemented using hardware, software, or a combination of hardware and software. The database 4520 can be local to the data processing system 4506. In some cases, the database 4520 can be remote from the data processing system 4506. The database 4520 can be accessed by one or more components of the data processing system 4506. The one or more components of the data processing system 4506 may add, remove, or update data in the database 4520. The database 4520 can include a collected data storage 4522, footwear storage 4524, battery storage 4526, performance profile storage 4528, model storage 4530, and gesture storage 4532.

The collected data storage 4522 can include, store, or maintain data collected from the one or more components of the system 4500, such as from the client device 4504 or the exoskeleton 100. The collected data storage 4522 can be accessed by the data collector 4510. For example, the data collector 4510 can collect data from the one or more components of the system 4500. Subsequently, the data collector 4510 can store or update the collected data in the collected data storage 4522. In some cases, the data collector 4510 or other components of the data processing system 4506 can remove or discard one or more outdated data from the collected data storage 4522 (or any storage of the database 4520).

The footwear storage 4524 can include, store, or maintain information associated with various footwear (e.g., footwear 600 or shoes). For example, the footwear storage 4524 can store a list of footwear available either for purchase by the subject or for inserting the footplate. The footwear storage 4524 can be accessed by the footplate selector 4512 or the data collector 4510 to identify the type of footwear or the one or more characteristics associated with the footwear. In some cases, the data processing system 4506 can communicate with an external database 4520 to update the list of footwear included in the footwear storage 4524. The footwear storage 4524 can be updated periodically (e.g., daily, weekly, or monthly) or aperiodically.

The battery storage 4526 can include, store, or maintain an indication of the types of battery cell. The battery storage 4526 can include at least one of a respective capacity or a maximum peak current associated with each type of battery cell. Further, the battery storage 4526 can include an indication of the associated performance profile to utilize based on the number of battery cells detected, for instance, by the data collector 4510.

The performance profile storage 4528 can include, store, or maintain the various performance profiles. The performance profile storage 4528 can be accessed by at least the performance configurator 4514, among other components of the data processing system 4506. For instance, the performance profile storage 4528 can be accessed by the performance configurator 4514 to select at least one performance profile to control or optimize the performance of the exoskeleton 100. Each performance profile can be configured to optimize at least one of the lifetime of the battery relative to the energy output or optimize the energy relative to the lifetime of the battery, for example. Each performance profile can be associated with a respective duty cycle and maximum energy output, for example.

The model storage 4530 can include, store, or maintain various models. The individual models can be associated with at least one of a respective characteristic or the mode for augmentation. The model can include one or more commands with which to instruct the electric motor of the exoskeleton 100. For example, the model can indicate the amount of torque to generate or force to apply to the footplate (e.g., via an axis of rotation about an ankle to augment motion). The model storage 4530 can be accessed by at least one of the model manager 4516 or the motor controller 4518. For example, the model manager 4516 can access the model storage 4530 to select, update, or otherwise manage the one or more models. The motor controller 4518 may access the model storage 4530 to obtain instructions or commands associated with the model, such as to instruct or control the electric motor.

The gesture storage 4532 can include, store, or maintain a list of gestures. Each gesture from the list of gestures can be associated with at least one respective command. Based on a detection of the gesture, the motor controller 4518 can execute the command to instruct the electric motor in accordance with the command. The list of gestures can be updated by the administrator or the subject. The command corresponding to each gesture may be updated or configure by the administrator or the subject.

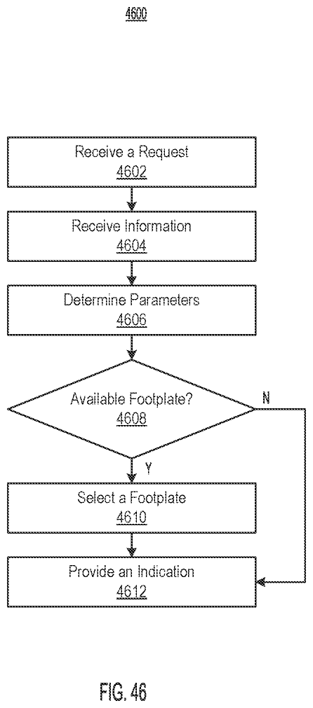

Referring now to FIG. 46 is an example flow diagram of a method 4600 for a footwear attachment process. The example method 4600 can be executed, performed, or otherwise carried out by one or more components of the system 4500 (e.g., data processing system 4506, client device 4504, exoskeleton 100, etc.) or any other devices or components described herein in conjunction with FIGS. 1-45. The method 4600 can include receiving a request, at ACT 4602. At ACT 4604, the method 4600 can include receiving information. At ACT 4606, the method 4600 can include determining parameters. At ACT 4608, the method 4600 can include determining whether the footplate is available. At ACT 4610, the method 4600 can include selecting a footplate. At ACT 4612, the method 4600 can include providing an indication.

Still referring to FIG. 46, and in further detail, at ACT 4602, the data processing system can receive a request to modify a footwear for augmentation. The footwear can include a sole formed of an insole and an outsole (e.g., an upper portion and a lower portion of the sole).

ACT 4604, the data processing system can receive information related to the selection of the footplate. For example, the data processing system can receive an indication of a type of footwear, such as at least one of a running footwear, a hiking footwear, a cross-training footwear, a basketball footwear, a boot, or a dress footwear, among others. The data processing system can receive information regarding at least one type of activity to be performed with the footwear (or the exoskeleton). The activity can include at least one of running, walking, or hiking, etc. In some cases, the type of footwear can indicate the type of activity to be performed.

At ACT 4606, the data processing system can determine the parameters associated with the footplate for selection. For example, the data processing system can select the footplate based on at least one of the type of footwear or the activity to be performed. The data processing system can determine a length for the first portion of the footplate and a length for the second portion of the footplate. The data processing system can determine an amount of rigidity for the second portion of the footplate based on the type of the footwear and the activity. The footplate can terminate behind a metatarsal joint of a foot on which the footwear is worn.

At ACT 4608, the data processing system can determine whether the footplate (e.g., with the determined parameters) is available for selection. The data processing system can perform a lookup in the database or inventory to determine whether the footplate is available or can be provided to the footwear. If the footplate is not available, the data processing system can proceed to ACT 4612. Otherwise, the data processing system can proceed to ACT 4610 to select the footplate.

At ACT 4610, the data processing system can select the footplate having the one or more determined parameters. For example, the data processing system can select, for insertion into the sole, the footplate having the determined length for the first portion of the footplate, the determined length for the second portion of the footplate, and the determined amount of rigidity for the second portion. The data processing system can select the second portion of the footplate that is thinner than the first portion of the footplate. The second portion of the footplate may at least partially overlaps with the first portion of the footplate.

In some cases, the data processing system can select, based on a type of the footwear or an activity to be performed with the footwear, the mounting component configured to protrude from a rear of the footwear. The data processing system can select, based on a type of the footwear or an activity to be performed with the footwear. The mounting component can be configured to protrude from a side at a heel of the footwear that is different from a rear of the footwear. The data processing system can select the actuator configured to couple to a lower limb of a subject wearing the footwear. The actuator can be configured to couple below a knee of the lower limb of the subject.

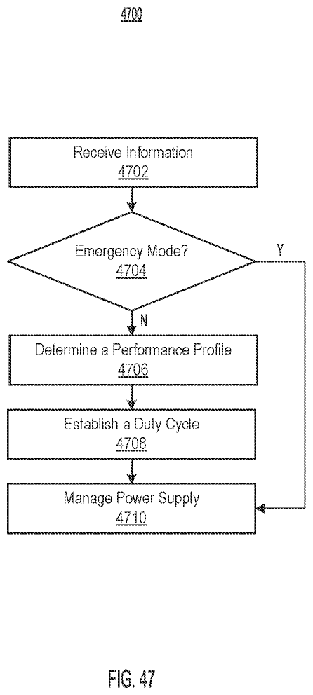

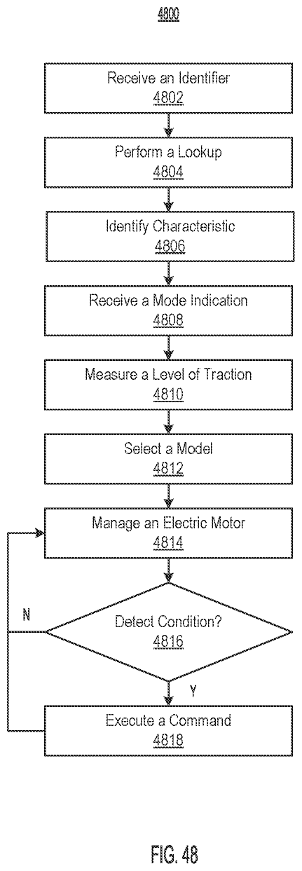

FIG. 47 is an example flow diagram of a method 4700 for battery and hardware for augmented motion. The example method 4700 can be executed, performed, or otherwise carried out by one or more components of the system 4500 (e.g., data processing system 4506, client device 4504, exoskeleton 100, etc.) or any other devices or components described herein in conjunction with FIGS. 1-45. The method 4700 can include receiving information, at ACT 4702. At ACT 4704, the method 4700 can include determining whether to enter into an emergency mode. At ACT 4706, the method 4700 can include determining a performance profile. At ACT 4708, the method 4700 can include establishing a duty cycle. At ACT 4710, the method 4700 can include managing the power supply.

Still referring to FIG. 47, and in further detail, at ACT 4702, the controller (e.g., the data processing system) can receive information associated with the performance of the motor or the battery of the exoskeleton for managing power supplied to the exoskeleton. For example, the controller can receive an indication of the type of battery cell(s) included in the battery pack. The type of battery cell can indicate at least one of a capacity or a maximum peak current. The controller may receive an indication of the type of activity to be performed or the type of footwear (e.g., one or more characteristics of the footwear) to use for determining the performance profile to select. The controller can receive an indication of the number of battery cells per battery pack, or the number of battery packs electrically connected to the controller or the electric motor.

At ACT 4704, the controller can determine whether to enter into an emergency mode. The controller can perform the determination or obtain an indication to enter the emergency mode at any point during the operation of the exoskeleton. For example, the controller can receive, by the controller, an indication to enter an emergency mode. The controller can allow, responsive to the indication, the switched-mode power supply to discharge the battery pack beyond a minimum limit established for the battery pack. Allowing the discharge of the battery pack beyond the minimum limit can be a part of a performance profile. Hence, the controller can proceed to manage the power supply in response to receiving an indication to enter the emergency mode. Otherwise, the controller can proceed to ACT 4706.

At ACT 4706, the controller can determine or select the performance profile. For example, the controller, electrically connected with the electric motor and the battery pack, can determine a performance profile for the electric motor based on a number of battery cells in the plurality of battery cells or a type of battery cell in the plurality of battery cells. With a greater number of battery cells or the type of battery cell that has a higher capacity of maximum peak current, a higher performance profile can be selected, for example.

In some case, the controller may detect that the battery pack has been replaced with a second battery pack including a second plurality of battery cells. The controller can determine a second performance profile for the electric motor based on a second number of battery cells in the second plurality of battery cells or a second type of battery cell in the second plurality of battery cells. In various aspects, the performance profile can be configured to optimize lifetime relative to energy output, or optimize energy relative to lifetime.

At ACT 4708, the controller can establish a duty cycle for a switched-mode power supply to cause the switched-mode power supply to convey power from the battery pack to the electric motor to generate the torque about the axis in accordance with the determined (e.g., first) performance profile. In some cases, the controller may establish a second duty cycle for the switched-mode power supply to cause the switched-mode power supply to convey power from the second battery pack to the electric motor to generate the torque about the axis in accordance with the determined second performance profile. The second performance profile can be different from the (e.g., first) performance profile.

At ACT 4710, the controller can cause the switched-mode power supply to discharge the battery pack based on the established duty cycle. For example, a second battery pack can be provided, which includes a second group of battery cells electrically connected to the electric motor and the controller. The second battery pack can include a greater number of battery cells relative to the battery pack. In this case, the controller can cause the switched-mode power supply to discharge the battery pack with the duty cycle greater than a threshold in accordance with the performance profile. In some case, the controller can select a second performance profile for the second battery pack. Accordingly, the controller can cause the switched-mode power supply to discharge the battery pack with a second duty cycle less than the threshold in accordance with the second performance profile.

FIG. 48 is an example flow diagram of a method 4800 for software and controller for augmented motion. The example method 4800 can be executed, performed, or otherwise carried out by one or more components of the system 4500 (e.g., data processing system 4506, client device 4504, exoskeleton 100, etc.) or any other devices or components described herein in conjunction with FIGS. 1-45. The method 4800 can include receiving an identifier, at ACT 4802. At ACT 4804, the method 4800 can include performing a lookup. At ACT 4806, the method 4800 can include identifying one or more characteristics. At ACT 4808, the method 4800 can include receiving an indication of a mode of augmentation. At ACT 4810, the method 4800 can include measuring the level of traction. At ACT 4812, the method 4800 can include selecting a model. At ACT 4814, the method 4800 can include managing an electric motor. At ACT 4816, the method 4800 can include detecting at least one condition. At ACT 4818, the method 4800 can include executing at least one command.

Still referring to FIG. 48, and in further detail, at ACT 4802, the controller can receive an identifier for the footwear. The controller can receive the identifier from the subject (e.g., via a user interface from the client device) or from the administrator managing the exoskeleton.

At ACT 4804, the controller can perform a lookup in a database with the identifier to identify the one or more characteristics associated with the footwear. For instance, the controller can determine, based on a lookup performed with an identifier for the footwear in a database, a first level of traction of an outsole of the footwear and a first level of rigidity of the sole (e.g., for a new footwear having an expected or predetermined characteristic(s)).

At ACT 4806, the controller, electrically connected with the electric motor and the battery pack, can identify one or more characteristics of the footwear in which the footplate is inserted. The one or more characteristics can include at least one of a thickness of the sole, a grip of an outsole of the footwear, a softness of the sole, or a rigidity of the sole, for example.

At ACT 4808, the controller can receive, via the user interface from the client device, an indication of a mode for augmentation. The mode for augmentation can include at least one of running, hiking, walking, launch mode, encouragement mode, or cast mode.

At ACT 4810, in some cases, the controller can measure, via an ankle angle sensor and torque applied by the electric motor, a second level of traction of the outsole and a second level of rigidity of the sole. The second level of traction may be less than the first level of traction. The second level of rigidity may be less than the first level of rigidity. The first level of traction and rigidity can be included in the database associated with the identifier of the footwear.

At ACT 4812, the controller can select a model for torque generation based on the one or more characteristics of the footwear. In some cases, the controller can select the model based on the one or more characteristics and the mode for augmentation. The controller can select, based on the second level of traction and the second level of rigidity, a second model with which to instruct the electric motor to generate torque.

At ACT 4814, the controller can instruct or command the electric motor to generate torque based on the model. In some cases, the controller can instruct the electric motor based on the second model, or any other models (or performance profiles) to operate the exoskeleton or apply force to the footplate.

At ACT 4816, the controller can detect one or more conditions to adjust one or more settings or configurations of at least one of the battery pack (e.g., energy output), the controller (e.g., clock rate or processing speed), or the motor (e.g., torque generation). For example, the controller can detect, via a sensor, a level of activity of the subject. Based on the level of activity less than a threshold, the controller can decrease a clock rate of the one or more processors of the controller (e.g., ACT 4818).

In some cases, the controller detect a rate of motion of the subject is less than a threshold. In this case, the controller can instruct, such as in the encouragement mode of augmentation, the electric motor to vibrate responsive to the detection of the rate of motion less than the threshold (e.g., ACT 4818). In some other cases, the controller may detect, via a sensor that monitors motions of the ankle joint, a gesture performed by the subject. The controller can perform a lookup with the detected gesture to identify a command corresponding to the gesture. The controller can execute the command to instruct the electric motor in accordance with the command (e.g., ACT 4818). In various aspects, if the controller does not detect any condition, such as no model selection, gestures, or monitoring of the activity, the controller can continue to manage the electric motor at ACT 4814, such as until an exit condition (e.g., terminating the operation of the electric motor).

### Further Examples

The following examples pertain to further embodiments, from which numerous permutations and configurations will be apparent.

Example 1 includes a method of integrating bipedalism augmentation components, comprising: providing footwear comprising a sole formed of an insole and an outsole; selecting, based on a size of the footwear, a footplate having a length that is less than the size of the footwear, the footplate comprising: a first portion of the footplate to extend along at least a first portion of the sole; a second portion of the footplate to extend along at least a second portion of the sole, wherein the first portion of the footplate has greater rigidity than the second portion of the footplate; and a mounting component connected to the first portion of the footplate; inserting the footplate between the insole and the outsole of the footwear such that the first portion of the footplate with greater rigidity is located proximate to a heel of the footwear and the mounting component protrudes at least partially from the footwear; and coupling a bracket to the mounting component, the bracket configured to receive force from an actuator and apply the force to the first portion of the footplate via an axis of rotation about an ankle to augment motion.

Example 2 includes the subject matter of Example 1, comprising: identifying, by a data processing system comprising one or more processors and memory, a type of the footwear; determining, by the data processing system prior to insertion, the length for the footplate based on the type of the footwear; and selecting, by the data processing system, the footplate with the length for insertion into the sole of the footwear.

Example 3 includes the subject matter of any of Examples 1 and 2, wherein the type of the footwear comprises at least one of a running footwear, a hiking footwear, a cross-training footwear, a basketball footwear, a boot, or a dress footwear.

Example 4 includes the subject matter of any of Examples 1 through 3, comprising: determining, by the data processing system based on the type of the footwear, the length of the footplate such that the footplate terminates behind a metatarsal joint of a foot on which the footwear is worn.

Example 5 includes the subject matter of any of Examples 1 through 4, comprising: identifying, by a data processing system comprising one or more processors and memory, an activity to be performed with the footwear; determining, by the data processing system prior to insertion, the length for the footplate based on the activity; and selecting, by the data processing system, the footplate with the length for insertion into the sole of the footwear.

Example 6 includes the subject matter of any of Examples 1 through 5, wherein the activity comprises at least one of running, walking, or hiking.

Example 7 includes the subject matter of any of Examples 1 through 6, comprising: identifying, by a data processing system comprising one or more processors and memory, a type of the footwear and an activity to be performed with the footwear; determining, by the data processing system prior to insertion, a length for the first portion of the footplate and a length for the second portion of the footplate; determining, by the data processing system prior to insertion, an amount of rigidity for the second portion of the footplate based on the type of the footwear and the activity; and selecting, by the data processing system for insertion into the sole, the footplate having the determined length for the first portion, the determined length for the second portion, and the determined amount of rigidity for the second portion.

Example 8 includes the subject matter of any of Examples 1 through 7, wherein the second portion of the footplate is thinner than the first portion of the footplate, and the second portion of the footplate at least partially overlaps with the first portion of the footplate.

Example 9 includes the subject matter of any of Examples 1 through 8, wherein the mounting component protrudes from a rear of the footwear.

Example 10 includes the subject matter of any of Examples 1 through 9, wherein the mounting component protrudes from a side at a heel of the footwear that is different from a rear of the footwear.

Example 11 includes the subject matter of any of Examples 1 through 10, comprising: coupling the actuator to a lower limb of a subject wearing the footwear, the actuator coupled below a knee of the lower limb of the subject.

Example 12 includes a system that integrates bipedalism augmentation components, comprising: a data processing system comprising memory and one or more processors to: receive a request to modify a footwear for augmentation, the footwear comprising a sole formed of an insole and an outsole; select a footplate based on a size of the footwear to have a length that is less than the size of the footwear, the footplate comprising: a first portion of the footplate to extend along at least a first portion of the sole; a second portion of the footplate to extend along at least a second portion of the sole, wherein the first portion of the footplate has greater rigidity than the second portion of the footplate; and a mounting component connected to the first portion of the footplate; provide an indication of the selected the footplate for insertion between the insole and the outsole of the footwear such that the first portion of the footplate with greater rigidity is located proximate to a heel of the footwear and the mounting component protrudes at least partially from the sole, wherein the mounting component is configured to couple to a bracket to receive force from an actuator and apply the force to the first portion of the footplate via an axis of rotation about an ankle to augment motion.

Example 13 includes the subject matter of Example 12, wherein the data processing system is further configured to select the footplate with the length based on a type of the footwear, the type of the footwear comprises at least one of a running footwear, a hiking footwear, a cross-training footwear, a basketball footwear, a boot, or a dress footwear.

Example 14 includes the subject matter of any of Examples 12 and 13, wherein the footplate terminates behind a metatarsal joint of a foot on which the footwear is worn.

Example 15 includes the subject matter of any of Examples 12 through 14, comprising the data processing system to select the footplate with a length based on an activity to be performed with the footwear, the activity comprising at least one of running, walking, or hiking.

Example 16 includes the subject matter of any of Examples 12 through 15, wherein the data processing system is further configured to: receive an indication of a type of the footwear and an activity to be performed with the footwear; determine, prior to insertion, a length for the first portion of the footplate and a length for the second portion of the footplate; determine, prior to insertion, an amount of rigidity for the second portion of the footplate based on the type of the footwear and the activity; and select, for insertion into the sole, the footplate having the determined length for the first portion of the footplate, the determined length for the second portion of the footplate, and the determined amount of rigidity for the second portion.

Example 17 includes the subject matter of any of Examples 12 through 16, wherein the data processing system is further configured to select the second portion of the footplate that is thinner than the first portion of the footplate, wherein the second portion of the footplate at least partially overlaps with the first portion of the footplate.

Example 18 includes the subject matter of any of Examples 12 through 17, wherein the data processing system is further configured to select, based on a type of the footwear or an activity to be performed with the footwear, the mounting component configured to protrude from a rear of the footwear.

Example 19 includes the subject matter of any of Examples 12 through 18, wherein the data processing system is further configured to select, based on a type of the footwear or an activity to be performed with the footwear, the mounting component configured to protrude from a side at a heel of the footwear that is different from a rear of the footwear.

Example 20 includes the subject matter of any of Examples 12 through 19, wherein the data processing system is further configured to select the actuator configured to couple to a lower limb of a subject wearing the footwear, the actuator configured to couple below a knee of the lower limb of the subject.

Example 21 includes a system for augmented bipedalism, comprising: a shin pad configured to couple to a shin of a subject below a knee of the subject; a battery pack comprising a plurality of battery cells; a footplate inserted in a sole of footwear; a housing, coupled to the shin pad, that encloses an electric motor configured to apply force via a lever to the footplate to generate torque about an axis of rotation of an ankle joint of the subject; and a controller comprising one or more processors and memory, electrically connected with the electric motor and the battery pack, to: determine a performance profile for the electric motor based on a number of battery cells in the plurality of battery cells or a type of battery cell in the plurality of battery cells; and establish a duty cycle for a switched-mode power supply to cause the switched-mode power supply to convey power from the battery pack to the electric motor to generate the torque about the axis in accordance with the determined performance profile.

Example 22 includes the subject matter of Example 21, wherein the battery pack is integrated in the shin pad.

Example 23 includes the subject matter of any of Examples 21 and 22, wherein the battery pack is flexible battery and configured to couple to a calf behind the shin.

Example 24 includes the subject matter of any of Examples 21 through 23, wherein the battery pack comprises a printed circuit board that flexes with the battery pack, the printed circuit board to connect each battery cell of the plurality of battery cells in series.

Example 25 includes the subject matter of any of Examples 21 through 24, wherein the battery pack is flexible and comprises a mechanical structure with flexible members configured to allow the battery pack to flex along a plurality of curved radii.

Example 26 includes the subject matter of any of Examples 21 through 25, wherein the battery pack comprises a first connector to electrically connect the battery pack to the electric motor, and a second connector to electrically connect to a charger.

Example 27 includes the subject matter of any of Examples 21 through 26, wherein the controller is further configured to: receive an indication to enter an emergency mode; and allow, responsive to the indication, the switched-mode power supply to discharge the battery pack beyond a minimum limit established for the battery pack.

Example 28 includes the subject matter of any of Examples 21 through 27, wherein the controller is further configured to: detect that the battery pack has been replaced with a second battery pack comprising a second plurality of battery cells; determine a second performance profile for the electric motor based on a second number of battery cells in the second plurality of battery cells or a second type of battery cell in the second plurality of battery cells; and establish a second duty cycle for the switched-mode power supply to cause the switched-mode power supply to convey power from the second battery pack to the electric motor to generate the torque about the axis in accordance with the determined second performance profile, wherein the second performance profile is different from the performance profile.

Example 29 includes the subject matter of any of Examples 21 through 28, wherein: the type of battery cell indicates at least one of a capacity or a maximum peak current; and the performance profile is configured to optimize lifetime relative to energy output, or optimize energy relative to lifetime.

Example 30 includes the subject matter of any of Examples 21 through 29, comprising: a second battery pack comprising a second plurality of battery cells electrically connected with the electric motor and the controller, wherein the second battery pack comprises a greater number of battery cells relative to the battery pack, wherein the controller is further configured to: cause the switched-mode power supply to discharge the battery pack with the duty cycle greater than a threshold in accordance with the performance profile; select a second performance profile for the second battery pack; and cause the switched-mode power supply to discharge the battery pack with a second duty cycle less than the threshold in accordance with the second performance profile.

Example 31 includes the subject matter of any of Examples 21 through 30, wherein a lifetime of the battery pack is less than a second lifetime of the second battery pack.

Example 32 includes the subject matter of any of Examples 21 through 31, wherein the controller is further configured to: receive an indication of an activity to be performed by the subject; and select one of the battery pack associated with the performance profile, or the second battery pack associated with the second performance profile, to deliver power to the electric motor to augment the activity.

Example 33 includes the subject matter of any of Examples 21 through 32, comprising: a second battery pack electrically connected to a second motor to provide torque to apply force via a second lever to a second footplate to generate torque about an axis of rotation of a second ankle joint of the subject, wherein the controller is further configured to balance, responsive to the battery pack electrically connected with the second battery pack, a state of charge of the battery pack with a state of charge of the second battery pack.

Example 34 includes a method for augmenting bipedalism, comprising: providing a shin pad configured to couple to a shin of a subject below a knee of the subject; providing a battery pack comprising a plurality of battery cells; providing a footplate inserted in a sole of footwear; providing a housing, coupled to the shin pad, that encloses an electric motor configured to apply force via a lever to the footplate to generate torque about an axis of rotation of an ankle joint of the subject; determining, by a controller comprising one or more processors and memory, electrically connected with the electric motor and the battery pack, a performance profile for the electric motor based on a number of battery cells in the plurality of battery cells or a type of battery cell in the plurality of battery cells; and establishing, by the controller, a duty cycle for a switched-mode power supply to cause the switched-mode power supply to convey power from the battery pack to the electric motor to generate the torque about the axis in accordance with the determined performance profile.

Example 35 includes the subject matter of Example 34 wherein the battery pack is integrated in the shin pad.

Example 36 includes the subject matter of any of Examples 34 and 35, wherein the battery pack is flexible battery and configured to couple to a calf behind the shin.

Example 37 includes the subject matter of any of Examples 34 through 36, comprising: receiving, by the controller, an indication to enter an emergency mode; and allowing, by the controller responsive to the indication, the switched-mode power supply to discharge the battery pack beyond a minimum limit established for the battery pack.

Example 38 includes the subject matter of any of Examples 34 through 37, comprising: detecting, by the controller, that the battery pack has been replaced with a second battery pack comprising a second plurality of battery cells; determining, by the controller, a second performance profile for the electric motor based on a second number of battery cells in the second plurality of battery cells or a second type of battery cell in the second plurality of battery cells; and establishing, by the controller, a second duty cycle for the switched-mode power supply to cause the switched-mode power supply to convey power from the second battery pack to the electric motor to generate the torque about the axis in accordance with the determined second performance profile, wherein the second performance profile is different from the performance profile.

Example 39 includes the subject matter of any of Examples 34 through 38, wherein: the type of battery cell indicates at least one of a capacity or a maximum peak current; and the performance profile is configured to optimize lifetime relative to energy output, or optimize energy relative to lifetime.

Example 40 includes the subject matter of any of Examples 34 through 39, comprising: providing a second battery pack comprising a second plurality of battery cells electrically connected with the electric motor and the controller, wherein the second battery pack comprises a greater number of battery cells relative to the battery pack, causing, by the controller, the switched-mode power supply to discharge the battery pack with the duty cycle greater than a threshold in accordance with the performance profile; selecting, by the controller, a second performance profile for the second battery pack; and causing, by the controller, the switched-mode power supply to discharge the battery pack with a second duty cycle less than the threshold in accordance with the second performance profile.

Example 41 includes a system for augmented bipedalism, comprising: a shin pad configured to couple to a shin of a subject below a knee of the subject; a battery pack; a footplate inserted in a sole of a footwear; a housing, coupled to the shin pad, that encloses an electric motor configured to apply force via a lever to the footplate to generate torque about an axis of rotation of an ankle joint of the subject; and a controller comprising one or more processors and memory, electrically connected with the electric motor and the battery pack, to: identify one or more characteristics of the footwear in which the footplate is inserted; receive, via a user interface, an indication of a mode for augmentation; select a model for torque generation based on the one or more characteristics of the footwear; and instruct the electric motor to generate torque based on the model.

Example 42 includes the subject matter of Example 41, wherein the one or more characteristics comprise at least one of a thickness of the sole, a grip of an outsole of the footwear, a softness of the sole, or a rigidity of the sole.

Example 43 includes the subject matter of any of Examples 41 and 42, wherein the controller is further configured to: receive, via the user interface, an identifier for the footwear; and perform a lookup in a database with the identifier to identify the one or more characteristics.

Example 44 includes the subject matter of any of Examples 41 through 43, wherein the controller is further configured to: select the model based on the one or more characteristics and the mode for augmentation, wherein the mode for augmentation comprises at least one of running, hiking, walking, launch mode, encouragement mode, or cast mode.

Example 45 includes the subject matter of any of Examples 41 through 44, wherein the controller is further configured to: determine, based on a lookup performed with an identifier for the footwear in a database, a first level of traction of an outsole of the footwear and a first level of rigidity of the sole; measure, via an ankle angle sensor and torque applied by the electric motor, a second level of traction of the outsole and a second level of rigidity of the sole, wherein the second level of traction is less than the first level of traction and the second level of rigidity is less than the first level of rigidity; and select, based on the second level of traction and the second level of rigidity, a second model with which to instruct the electric motor to generate torque.

Example 46 includes the subject matter of any of Examples 41 through 45, wherein the controller is further configured to: receive, via the user interface, the one or more characteristics of the footwear.

Example 47 includes the subject matter of any of Examples 41 through 46, wherein the controller is further configured to: receive, via the user interface, the indication of the mode for augmentation comprising a performance mode configured to reduce a rate at which the electric motor transitions from augmented running to augmented walking in order to encourage running at a steady pace.

Example 48 includes the subject matter of any of Examples 41 through 47, wherein the controller is further configured to: set a clock rate of the one or more processors based on the mode for augmentation.

Example 49 includes the subject matter of any of Examples 41 through 48, wherein the controller is further configured to: detect, via a sensor, a level of activity of the subject; and increase a clock rate of the one or more processors based on the level of activity greater than a threshold.

Example 50 includes the subject matter of any of Examples 41 through 49, wherein the controller is further configured to: set a sample rate of one or more sensors of the system based on a level of activity of the subject.

Example 51 includes the subject matter of any of Examples 41 through 50, wherein the controller is further configured to: detect a rate of motion of the subject is less than a threshold; and instruct the electric motor to vibrate responsive to the detection of the rate of motion less than the threshold.

Example 52 includes the subject matter of any of Examples 41 through 51, wherein the controller is further configure to: detect, via a sensor that monitors motions of the ankle joint, a gesture performed by the subject; perform a lookup with the detected gesture to identify a command corresponding to the gesture; and execute the command to instruct the electric motor in accordance with the command.

Example 53 includes a method for augmenting bipedalism, comprising: providing a shin pad configured to couple to a shin of a subject below a knee of the subject; providing a battery pack; providing a footplate inserted in a sole of a footwear; providing a housing, coupled to the shin pad, that encloses an electric motor configured to apply force via a lever to the footplate to generate torque about an axis of rotation of an ankle joint of the subject; and identifying, by a controller comprising one or more processors and memory, electrically connected with the electric motor and the battery pack, one or more characteristics of the footwear in which the footplate is inserted; receiving, by the controller via a user interface, an indication of a mode for augmentation; selecting, by the controller, a model for torque generation based on the one or more characteristics of the footwear; and instructing, by the controller, the electric motor to generate torque based on the model.

Example 54 includes the subject matter of Example 53, wherein the one or more characteristics comprise at least one of a thickness of the sole, a grip of an outsole of the footwear, a softness of the sole, or a rigidity of the sole.

Example 55 includes the subject matter of any of Examples 53 and 54, comprising: receiving, by the controller via the user interface, an identifier for the footwear; and performing, by the controller, a lookup in a database with the identifier to identify the one or more characteristics.

Example 56 includes the subject matter of any of Examples 53 through 55, comprising: selecting, by the controller, the model based on the one or more characteristics and the mode for augmentation, wherein the mode for augmentation comprises at least one of running, hiking, walking, launch mode, encouragement mode, or cast mode.

Example 57 includes the subject matter of any of Examples 53 through 56, comprising: determining, by the controller based on a lookup performed with an identifier for the footwear in a database, a first level of traction of an outsole of the footwear and a first level of rigidity of the sole; measuring, by the controller via an ankle angle sensor and torque applied by the electric motor, a second level of traction of the outsole and a second level of rigidity of the sole, wherein the second level of traction is less than the first level of traction and the second level of rigidity is less than the first level of rigidity; and selecting, by the controller based on the second level of traction and the second level of rigidity, a second model with which to instruct the electric motor to generate torque.

Example 58 includes the subject matter of any of Examples 53 through 57, comprising: detecting, by the controller via a sensor, a level of activity of the subject; and decreasing, by the controller, a clock rate of the one or more processors based on the level of activity less than a threshold.

Example 59 includes the subject matter of any of Examples 53 through 58, comprising: detecting, by the controller, a rate of motion of the subject is less than a threshold; and instructing, by the controller, the electric motor to vibrate responsive to the detection of the rate of motion less than the threshold.

Example 60 includes the subject matter of any of Examples 53 through 59, comprising: detecting, by the controller via a sensor that monitors motions of the ankle joint, a gesture performed by the subject; performing, by the controller, a lookup with the detected gesture to identify a command corresponding to the gesture; and executing, by the controller, the command to instruct the electric motor in accordance with the command.

Embodiments of the subject matter and the operations described in this specification can be implemented in digital electronic circuitry, or in computer software, firmware, or hardware, including the structures disclosed in this specification and their structural equivalents, or in combinations of one or more of them. The subject matter described in this specification can be implemented as one or more computer programs, e.g., one or more circuits of computer program instructions, encoded on one or more computer storage media for execution by, or to control the operation of, data processing apparatus. Alternatively or in addition, the program instructions can be encoded on an artificially generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or be included in, one or more separate components or media (e.g., multiple CDs, disks, or other storage devices).

The operations described in this specification can be performed by a data processing apparatus on data stored on one or more computer-readable storage devices or received from other sources. The term “data processing apparatus” or “computing device” encompasses various apparatuses, devices, and machines for processing data, including by way of example a programmable processor, a computer, a system on a chip, or multiple ones, or combinations of the foregoing. The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand-alone program or as a circuit, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more circuits, subprograms, or portions of code). A computer program can be deployed to be executed on one computer or on multiple computers that are located at one site or distributed across multiple sites and interconnected by a communication network.

Processors suitable for the execution of a computer program include, by way of example, microprocessors, and any one or more processors of a digital computer. A processor can receive instructions and data from a read only memory or a random access memory or both. The elements of a computer are a processor for performing actions in accordance with instructions and one or more memory devices for storing instructions and data. A computer can include, or be operatively coupled to receive data from or transfer data to, or both, one or more mass storage devices for storing data, e.g., magnetic, magneto optical disks, or optical disks. A computer need not have such devices. Moreover, a computer can be embedded in another device, e.g., a personal digital assistant (PDA), a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

The implementations described herein can be implemented in any of numerous ways including, for example, using hardware, software or a combination thereof. When implemented in software, the software code can be executed on any suitable processor or collection of processors, whether provided in a single computer or distributed among multiple computers.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible format.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

A computer employed to implement at least a portion of the functionality described herein may comprise a memory, one or more processing units (also referred to herein simply as “processors”), one or more communication interfaces, one or more display units, and one or more user input devices. The memory may comprise any computer-readable media, and may store computer instructions (also referred to herein as “processor-executable instructions”) for implementing the various functionalities described herein. The processing unit(s) may be used to execute the instructions. The communication interface(s) may be coupled to a wired or wireless network, bus, or other communication means and may therefore allow the computer to transmit communications to or receive communications from other devices. The display unit(s) may be provided, for example, to allow a user to view various information in connection with execution of the instructions. The user input device(s) may be provided, for example, to allow the user to make manual adjustments, make selections, enter data or various other information, or interact in any of a variety of manners with the processor during execution of the instructions.

The various methods or processes outlined herein may be coded as software that is executable on one or more processors that employ any one of a variety of operating systems or platforms. Additionally, such software may be written using any of a number of suitable programming languages or programming or scripting tools, and also may be compiled as executable machine language code or intermediate code that is executed on a framework or virtual machine.

In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other non-transitory medium or tangible computer storage medium) encoded with one or more programs that, when executed on one or more computers or other processors, perform methods that implement the various embodiments of the solution discussed above. The computer readable medium or media can be transportable, such that the program or programs stored thereon can be loaded onto one or more different computers or other processors to implement various aspects of the present solution as discussed above.

The terms “program” or “software” are used herein to refer to any type of computer code or set of computer-executable instructions that can be employed to program a computer or other processor to implement various aspects of embodiments as discussed above. One or more computer programs that when executed perform methods of the present solution need not reside on a single computer or processor, but may be distributed in a modular fashion amongst a number of different computers or processors to implement various aspects of the present solution.

Computer-executable instructions may be in many forms, such as program modules, executed by one or more computers or other devices. Program modules can include routines, programs, objects, components, data structures, or other components that perform particular tasks or implement particular abstract data types. The functionality of the program modules can be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

Any references to implementations or elements or acts of the systems and methods herein referred to in the singular can include implementations including a plurality of these elements, and any references in plural to any implementation or element or act herein can include implementations including only a single element. References in the singular or plural form are not intended to limit the presently disclosed systems or methods, their components, acts, or elements to single or plural configurations. References to any act or element being based on any information, act or element may include implementations where the act or element is based at least in part on any information, act, or element.

Any implementation disclosed herein may be combined with any other implementation, and references to “an implementation,” “some implementations,” “an alternate implementation,” “various implementations,” “one implementation” or the like are not necessarily mutually exclusive and are intended to indicate that a particular feature, structure, or characteristic described in connection with the implementation may be included in at least one implementation. Such terms as used herein are not necessarily all referring to the same implementation. Any implementation may be combined with any other implementation, inclusively or exclusively, in any manner consistent with the aspects and implementations disclosed herein.

References to “or” may be construed as inclusive so that any terms described using “or” may indicate any of a single, more than one, and all of the described terms. References to at least one of a conjunctive list of terms may be construed as an inclusive OR to indicate any of a single, more than one, and all of the described terms. For example, a reference to “at least one of ‘A’ and ‘B’” can include only ‘A’, only ‘B’, as well as both ‘A’ and ‘B’. Elements other than ‘A’ and ‘B’ can also be included.

The systems and methods described herein may be embodied in other specific forms without departing from the characteristics thereof. The foregoing implementations are illustrative rather than limiting of the described systems and methods.

Where technical features in the drawings, detailed description or any claim are followed by reference signs, the reference signs have been included to increase the intelligibility of the drawings, detailed description, and claims. Accordingly, neither the reference signs nor their absence have any limiting effect on the scope of any claim elements.

The systems and methods described herein may be embodied in other specific forms without departing from the characteristics thereof. The foregoing implementations are illustrative rather than limiting of the described systems and methods. Scope of the systems and methods described herein is thus indicated by the appended claims, rather than the foregoing description, and changes that come within the meaning and range of equivalency of the claims are embraced therein.

1. A method of integrating bipedalism augmentation components, comprising:
providing footwear comprising a sole formed of an insole and an outsole;
selecting, based on a size of the footwear, a footplate having a length that is less than the size of the footwear, the footplate comprising:
a first portion of the footplate to extend along at least a first portion of the sole;
a second portion of the footplate to extend along at least a second portion of the sole, wherein the first portion of the footplate has greater rigidity than the second portion of the footplate; and
a mounting component connected to the first portion of the footplate;
inserting the footplate between the insole and the outsole of the footwear such that the first portion of the footplate with greater rigidity is located proximate to a heel of the footwear and the mounting component protrudes at least partially from the footwear; and
coupling a bracket to the mounting component, the bracket configured to receive force from an actuator and apply the force to the first portion of the footplate via an axis of rotation about an ankle to augment motion.

2. The method of claim 1, comprising:
identifying, by a data processing system comprising one or more processors and memory, a type of the footwear;
determining, by the data processing system prior to insertion, the length for the footplate based on the type of the footwear; and
selecting, by the data processing system, the footplate with the length for insertion into the sole of the footwear.

3. The method of claim 2, wherein the type of the footwear comprises at least one of a running footwear, a hiking footwear, a cross-training footwear, a basketball footwear, a boot, or a dress footwear.

4. The method of claim 2, comprising:
determining, by the data processing system based on the type of the footwear, the length of the footplate such that the footplate terminates behind a metatarsal joint of a foot on which the footwear is worn.

5. The method of claim 1, comprising:
identifying, by a data processing system comprising one or more processors and memory, an activity to be performed with the footwear;
determining, by the data processing system prior to insertion, the length for the footplate based on the activity; and
selecting, by the data processing system, the footplate with the length for insertion into the sole of the footwear.

6. The method of claim 5, wherein the activity comprises at least one of running, walking, or hiking.

7. The method of claim 1, comprising:
identifying, by a data processing system comprising one or more processors and memory, a type of the footwear and an activity to be performed with the footwear;
determining, by the data processing system prior to insertion, a length for the first portion of the footplate and a length for the second portion of the footplate;
determining, by the data processing system prior to insertion, an amount of rigidity for the second portion of the footplate based on the type of the footwear and the activity; and
selecting, by the data processing system for insertion into the sole, the footplate having the determined length for the first portion, the determined length for the second portion, and the determined amount of rigidity for the second portion.

8. The method of claim 1, wherein the second portion of the footplate is thinner than the first portion of the footplate, and the second portion of the footplate at least partially overlaps with the first portion of the footplate.

9. The method of claim 1, wherein the mounting component protrudes from a rear of the footwear.

10. The method of claim 1, wherein the mounting component protrudes from a side at a heel of the footwear that is different from a rear of the footwear.

11. The method of claim 1, comprising:
coupling the actuator to a lower limb of a subject wearing the footwear, the actuator coupled below a knee of the lower limb of the subject.

12. A system that integrates bipedalism augmentation components, comprising:
a data processing system comprising memory and one or more processors to:
receive a request to modify a footwear for augmentation, the footwear comprising a sole formed of an insole and an outsole;
select a footplate based on a size of the footwear to have a length that is less than the size of the footwear, the footplate comprising:
a first portion of the footplate to extend along at least a first portion of the sole;
a second portion of the footplate to extend along at least a second portion of the sole, wherein the first portion of the footplate has greater rigidity than the second portion of the footplate; and
a mounting component connected to the first portion of the footplate;
provide an indication of the selected the footplate for insertion between the insole and the outsole of the footwear such that the first portion of the footplate with greater rigidity is located proximate to a heel of the footwear and the mounting component protrudes at least partially from the sole,
wherein the mounting component is configured to couple to a bracket to receive force from an actuator and apply the force to the first portion of the footplate via an axis of rotation about an ankle to augment motion.

13. The system of claim 12, wherein the data processing system is further configured to select the footplate with the length based on a type of the footwear, the type of the footwear comprises at least one of a running footwear, a hiking footwear, a cross-training footwear, a basketball footwear, a boot, or a dress footwear.

14. The system of claim 13, wherein the footplate terminates behind a metatarsal joint of a foot on which the footwear is worn.

15. The system of claim 12, comprising the data processing system to select the footplate with a length based on an activity to be performed with the footwear, the activity comprising at least one of running, walking, or hiking.

16. The system of claim 12, wherein the data processing system is further configured to:
receive an indication of a type of the footwear and an activity to be performed with the footwear;
determine, prior to insertion, a length for the first portion of the footplate and a length for the second portion of the footplate;
determine, prior to insertion, an amount of rigidity for the second portion of the footplate based on the type of the footwear and the activity; and
select, for insertion into the sole, the footplate having the determined length for the first portion of the footplate, the determined length for the second portion of the footplate, and the determined amount of rigidity for the second portion.

17. The system of claim 12, wherein the data processing system is further configured to select the second portion of the footplate that is thinner than the first portion of the footplate, wherein the second portion of the footplate at least partially overlaps with the first portion of the footplate.

18. The system of claim 12, wherein the data processing system is further configured to select, based on a type of the footwear or an activity to be performed with the footwear, the mounting component configured to protrude from a rear of the footwear.

19. The system of claim 12, wherein the data processing system is further configured to select, based on a type of the footwear or an activity to be performed with the footwear, the mounting component configured to protrude from a side at a heel of the footwear that is different from a rear of the footwear.

20. The system of claim 12, wherein the data processing system is further configured to select the actuator configured to couple to a lower limb of a subject wearing the footwear, the actuator configured to couple below a knee of the lower limb of the subject.

21.-60. (canceled)
